SEQUENCE LISTING (27 sequences)

SEQ ID NO: 1 - DNA, 1248 bp, synthetic construct
  misc_feature 1..1248: note = coFIX-1-R338L
  source 1..1248: mol_type = other DNA
tacaatagtgggaagctggaggaatttgtgcagggcaacctggagagagaatgcatggaggaaaagtgtagcttcgaggaagcccgcgaggtgtttgaaaatacagagcgaaccacagagttctggaagcagtatgtggacggcgatcagtgcgagagcaacccctgtctgaatggcggaagttgcaaagacgatatcaactcatacgaatgctggtgtcctttcgggtttgaaggcaaaaattgcgagctggacgtgacatgtaacattaagaatggacggtgcgagcagttttgtaaaaactctgccgataataaggtggtgtgcagctgtactgaaggatatcgcctggctgagaaccagaagtcctgcgaaccagcagtgcccttcccttgtgggagggtgagcgtctcccagacttcaaaactgaccagagcagagacagtgtttcccgacgtggattacgtcaacagcactgaggccgaaaccatcctggacaacattactcagtctacccagagtttcaatgactttactcgggtggtcgggggcgaggatgctaaaccaggccagttcccctggcaggtggtcctgaacggaaaggtggatgcattttgcggagggtctatcgtgaatgagaaatggattgtcaccgccgctcactgcgtggaaaccggagtcaagatcacagtggtcgctggggagcacaacattgaggaaacagaacatactgagcagaagcggaatgtgatccgcatcattcctcaccataactacaatgcagccatcaacaaatacaatcatgacattgccctgctggaactggatgagcctctggtgctgaacagctacgtcactccaatctgcattgctgacaaagagtataccaatatcttcctgaagtttggatcagggtacgtgagcggctggggaagagtcttccacaagggcaggagcgccctggtgctccagtatctgcgagtgcctctggtcgatcgagctacctgtctgctctctaccaagtttacaatctacaacaacatgttctgcgctgggtttcacgagggaggacgagactcctgtcagggcgattctgggggcccacatgtgacagaggtcgaaggcaccagcttcctgactggcatcatttcctggggagaggaatgtgcaatgaagggaaaatacgggatctacaccaaagtgagccgctatgtgaactggatcaaggaaaaaaccaaactgacctaa

SEQ ID NO: 2 - DNA, 1386 bp, synthetic construct
  misc_feature 1..1386: note = coFIX-1b-R338L
  source 1..1386: mol_type = other DNA
atgcagagagtcaacatgattatggctgagtcacctgggctgattactatttgcctgctgggctacctgctgtccgccgagtgtaccgtgttcctggaccatgagaacgcaaataagatcctgaacaggcccaaaagatacaatagtgggaagctggaggaatttgtgcagggcaacctggagagagaatgcatggaggaaaagtgtagcttcgaggaagcccgcgaggtgtttgaaaatacagagcgaaccacagagttctggaagcagtatgtggacggcgatcagtgcgagagcaacccctgtctgaatggcggaagttgcaaagacgatatcaactcatacgaatgctggtgtcctttcgggtttgaaggcaaaaattgcgagctggacgtgacatgtaacattaagaatggacggtgcgagcagttttgtaaaaactctgccgataataaggtggtgtgcagctgtactgaaggatatcgcctggctgagaaccagaagtcctgcgaaccagcagtgcccttcccttgtgggagggtgagcgtctcccagacttcaaaactgaccagagcagagacagtgtttcccgacgtggattacgtcaacagcactgaggccgaaaccatcctggacaacattactcagtctacccagagtttcaatgactttactcgggtggtcgggggcgaggatgctaaaccaggccagttcccctggcaggtggtcctgaacggaaaggtggatgcattttgcggagggtctatcgtgaatgagaaatggattgtcaccgccgctcactgcgtggaaaccggagtcaagatcacagtggtcgctggggagcacaacattgaggaaacagaacatactgagcagaagcggaatgtgatccgcatcattcctcaccataactacaatgcagccatcaacaaatacaatcatgacattgccctgctggaactggatgagcctctggtgctgaacagctacgtcactccaatctgcattgctgacaaagagtataccaatatcttcctgaagtttggatcagggtacgtgagcggctggggaagagtcttccacaagggcaggagcgccctggtgctccagtatctgcgagtgcctctggtcgatcgagctacctgtctgctgtctaccaagtttacaatctacaacaacatgttctgcgctgggtttcacgagggaggacgagactcctgtcagggcgattctgggggcccacatgtgacagaggtcgaaggcaccagcttcctgactggcatcatttcctggggagaggaatgtgcaatgaagggaaaatacgggatctacaccaaagtgagccgctatgtgaactggatcaaggaaaaaaccaaactgacctaa

SEQ ID NO: 3 - DNA, 1386 bp, synthetic construct
  misc_feature 1..1386: note = coFIX-2-R338L
  source 1..1386: mol_type = other DNA
atgcagagggtgaacatgatcatggccgagagccccggcctgatcaccatctgcctgctgggctacctgctgagcgccgagtgcaccgtgttcctggaccacgagaacgccaacaagatcctgaacaggcccaagaggtacaacagcggcaagctggaggagttcgtgcagggcaacctggagagggagtgcatggaggagaagtgcagcttcgaggaggccagggaggtgttcgagaacaccgagaggaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggcaggtgcgagcagttctgcaagaacagcgccgacaacaaggtggtgtgcagctgcaccgagggctacaggctggccgagaaccagaagagctgcgagcccgccgtgcccttcccctgcggcagggtgagcgtgagccagaccagcaagctgaccagggccgagaccgtgttccccgacgtggactacgtgaacagcaccgaggccgagaccatcctggacaacatcacccagagcacccagagcttcaacgacttcaccagagtggtggggggcgaggacgccaagcccggccagttcccctggcaggtcgtgctgaatggcaaagtcgatgccttctgcgggggcagcatcgtcaacgagaagtggattgtgactgccgcccattgcgtggaaaccggggtgaagatcactgtggtggctggggagcacaacatcgaggaaaccgagcacaccgagcagaagaggaacgtgatcaggattatcccccatcacaactacaatgccgccatcaataagtacaaccatgatattgccctgctggagctggatgaacccctggtcctgaacagctatgtgactcccatctgcattgccgacaaggagtataccaacatcttcctgaaatttggcagcggctatgtctctggctggggcagggtgttccataaggggaggagcgccctggtcctgcagtacctgagagtgcccctggtggacagggccacctgcctgctgagcaccaagttcaccatctacaacaatatgttttgcgctggcttccatgaggggggcagggacagctgccagggggacagcgggggcccccatgtgactgaggtggagggcaccagcttcctgaccggcatcatcagctggggggaggagtgcgccatgaaggggaagtatggcatctacaccaaagtctccagatacgtcaactggatcaaggagaagaccaagctgacctaa

SEQ ID NO: 4 - DNA, 1386 bp, synthetic construct
  misc_feature 1..1386: note = coFIX-3-R338L
  source 1..1386: mol_type = other DNA
atgcagagggtgaacatgatcatggccgagagccccggcctgatcaccatctgcctgctgggctacctgctgagcgccgagtgcaccgtgttcctggaccacgagaacgccaacaagatcctgaacaggcccaagaggtacaacagcggcaagctggaggagttcgtgcagggcaacctggagagggagtgcatggaggagaagtgcagcttcgaggaggccagggaggtgttcgagaacaccgagaggaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggcaggtgcgagcagttctgcaagaacagcgccgacaacaaggtggtgtgcagctgcaccgagggctacaggctggccgagaaccagaagagctgcgagcccgccgtgcccttcccctgcggcagggtgagcgtgagccagaccagcaagctgaccagggccgagaccgtgttccccgacgtggactacgtgaacagcaccgaggccgagaccatcctggacaacatcacccagagcacccagagcttcaacgacttcaccagggtggtgggcggcgaggacgccaagcccggccagtttccctggcaggtggtgctgaacggcaaggtggacgccttctgcggcggcagcatcgtgaacgagaagtggatcgtgaccgctgcccattgcgtggaaaccggcgtgaagatcaccgtggtggccggcgagcacaacatcgaagagaccgagcacaccgaacagaaaaggaacgtgatcaggatcatccctcaccataactacaatgccgccattaacaagtacaatcacgacatcgctctgctggaactggatgaacccctggtgctgaacagctacgtgacccctatctgcatcgccgacaaggagtatactaacatctttctgaagtttggcagcggctatgtgagcggctggggcagggtgttccacaaaggcaggagcgccctggtgctgcagtacctgagggtgcccctggtggatagggctacctgcctgctgagcaccaagttcaccatctacaacaacatgttctgtgccggcttccacgaaggcggcagggactcttgccagggcgacagcggcggcccccatgtgaccgaggtggaaggcactagctttctgaccggcatcatcagctggggcgaggagtgcgccatgaagggcaagtacggcatctacactaaggtgagcaggtacgtgaactggatcaaagaaaagaccaagctgacctaa

SEQ ID NO: 5 - DNA, 1386 bp, synthetic construct
  misc_feature 1..1386: note = coFIX-4-R338L
  source 1..1386: mol_type = other DNA
atgcagagggtgaacatgatcatggccgagagccccggcctgatcaccatctgcctgctgggctacctgctgagcgccgagtgcaccgtgttcctggaccacgagaacgccaacaagatcctgaacaggcccaagaggtacaacagcggcaagctggaggagttcgtgcagggcaacctggagagggagtgcatggaggagaagtgcagcttcgaggaggccagggaggtgttcgagaacaccgagaggaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggcaggtgcgagcagttctgcaagaacagcgccgacaacaaggtggtgtgcagctgcaccgagggctacaggctggccgagaaccagaagagctgcgagcccgccgtgcccttcccctgcggcagggtgagcgtgagccagaccagcaagctgaccagggccgagaccgtgttccccgacgtggactacgtgaacagcaccgaggccgagaccatcctggacaacatcacccagagcacccagagcttcaacgacttcaccagggtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagaggaacgtgatcaggatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggcagggtgttccacaagggcaggagcgccctggtgctgcagtacctgagggtgcccctggtggacagggccacctgcctgctgagcaccaagttcaccatctacaacaacatgttctgcgccggcttccacgagggcggcagggacagctgccagggcgacagcggcggcccccacgtgaccgaggtggagggcaccagcttcctgaccggcatcatcagctggggcgaggagtgcgccatgaagggcaagtacggcatctacaccaaggtgagcaggtacgtgaactggatcaaggagaagaccaagctgacctaa

SEQ ID NO: 6 - DNA, 1386 bp, synthetic construct
  misc_feature 1..1386: note = coFIX-5-R338L
  source 1..1386: mol_type = other DNA
atgcagagggtcaacatgatcatggctgagtctcctggcctgatcaccatctgcctgctgggctatctgctgtccgctgagtgcactgtcttcctggaccacgagaacgccaacaagatcctgaacaggcccaagaggtataactctggcaagctggaggagtttgtgcaggggaacctggagagggagtgcatggaggagaagtgcagcttcgaggaggccagggaggtgtttgagaacactgagaggaccaccgagttctggaagcagtatgtggacggggaccagtgcgagtctaacccttgcctgaacgggggcagctgcaaggatgacatcaacagctatgagtgctggtgccctttcggcttcgagggcaagaactgcgagctggatgtgacctgcaacatcaagaacggcaggtgcgagcagttctgcaagaactctgccgacaacaaggtggtgtgcagctgcactgagggctataggctggctgagaaccagaagagctgtgagcctgctgtgcccttcccctgcggcagagtgtctgtgagccagaccagcaagctgaccagagctgagactgtcttccccgacgtggactatgtgaacagcaccgaggctgagaccatcctggacaacatcacccagtctacccagtctttcaacgacttcaccagagtggtggggggcgaggacgccaagcctggccagttcccctggcaggtcgtgctgaacggcaaagtggacgccttctgcgggggcagcatcgtcaacgagaagtggatcgtgactgctgctcactgcgtggaaaccggggtgaagatcaccgtggtggccggcgagcacaacatcgaggagaccgagcacaccgagcagaagaggaacgtgatcaggatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggcagggtgttccacaagggcaggagcgccctggtgctgcagtacctgagggtgcccctggtggacagggccacctgcctgctgagcaccaagttcaccatctacaacaacatgttctgcgccggcttccacgagggcggcagggacagctgccagggcgacagcggcggcccccacgtgaccgaggtggagggcaccagcttcctgaccggcatcatcagctggggcgaggagtgcgccatgaagggcaagtacggcatctacaccaaggtgagcaggtacgtgaactggatcaaggagaagaccaagctgacctaa

SEQ ID NO: 7 - DNA, 1386 bp, synthetic construct
  misc_feature 1..1386: note = coFIX-6-R338L
  source 1..1386: mol_type = other DNA
atgcagagggtcaacatgatcatggctgagtctcctggcctgatcactatctgcctgctgggctacctgctgagcgccgagtgcactgtcttcctggaccacgagaacgccaacaagatcctgaacaggcccaagagatacaactctggcaagctggaggagtttgtgcaggggaacctggagagggagtgcatggaggagaagtgcagcttcgaggaggccagggaggtgtttgagaacactgagaggaccactgagttctggaagcagtatgtggacggggaccagtgcgagtctaacccttgcctgaacgggggcagctgcaaggatgacatcaacagctacgagtgctggtgccctttcggcttcgagggcaagaactgcgagctggatgtgacttgcaacatcaagaacggcaggtgcgagcagttctgcaagaactctgccgacaacaaagtcgtgtgcagctgcactgagggctacagactggctgagaaccagaagagctgtgagcctgctgtgcccttcccctgcggcagagtgtctgtgagccagaccagcaagctgaccagagccgaaaccgtgttccccgacgtggactatgtgaacagcactgaggctgagaccatcctggacaacatcactcagtctacccagtctttcaacgacttcaccagagtggtggggggcgaggacgccaagcctggccagttcccctggcaggtcgtgctgaacggcaaggtggacgccttctgcgggggcagcatcgtcaacgagaagtggatcgtgactgccgcccactgcgtggagactggggtgaagatcactgtggtggctggggagcacaacatcgaggaaaccgagcacactgagcagaagaggaacgtgatcaggattatcccccaccacaactacaacgccgccatcaacaagtacaaccacgacatcgccctgctggagctggatgaacccctggtgctgaacagctacgtgacccctatctgcatcgccgacaaggagtacactaacatcttcctgaagttcggcagcggctacgtgagcggctggggcagggtgttccacaagggcaggagcgccctggtgctgcagtacctgagggtgcccctggtggacagggccacctgcctgctgagcaccaagttcaccatctacaacaacatgttctgcgccggcttccacgagggcggcagggacagctgccagggcgacagcggcggcccccacgtgaccgaggtggagggcaccagcttcctgaccggcatcatcagctggggcgaggagtgcgccatgaagggcaagtacggcatctacaccaaggtgagcaggtacgtgaactggatcaaggagaagaccaagctgacctaa

SEQ ID NO: 8 - DNA, 1389 bp, synthetic construct
  misc_feature 1..1389: note = factor IX nucleotide sequence
  source 1..1389: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaatctagagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttgataa

SEQ ID NO: 9 - DNA, 1389 bp, synthetic construct
  misc_feature 1..1389: note = coFIX-1
  source 1..1389: mol_type = other DNA
atgcagagagtcaacatgattatggctgagtcacctgggctgattactatttgcctgctgggctacctgctgtccgccgagtgtaccgtgttcctggaccatgagaacgcaaataagatcctgaacaggcccaaaagatacaatagtgggaagctggaggaatttgtgcagggcaacctggagagagaatgcatggaggaaaagtgtagcttcgaggaagcccgcgaggtgtttgaaaatacagagcgaaccacagagttctggaagcagtatgtggacggcgatcagtgcgagagcaacccctgtctgaatggcggaagttgcaaagacgatatcaactcatacgaatgctggtgtcctttcgggtttgaaggcaaaaattgcgagctggacgtgacatgtaacattaagaatggacggtgcgagcagttttgtaaaaactctgccgataataaggtggtgtgcagctgtactgaaggatatcgcctggctgagaaccagaagtcctgcgaaccagcagtgcccttcccttgtgggagggtgagcgtctcccagacttcaaaactgaccagagcagagacagtgtttcccgacgtggattacgtcaacagcactgaggccgaaaccatcctggacaacattactcagtctacccagagtttcaatgactttactcgggtggtcgggggcgaggatgctaaaccaggccagttcccctggcaggtggtcctgaacggaaaggtggatgcattttgcggagggtctatcgtgaatgagaaatggattgtcaccgccgctcactgcgtggaaaccggagtcaagatcacagtggtcgctggggagcacaacattgaggaaacagaacatactgagcagaagcggaatgtgatccgcatcattcctcaccataactacaatgcagccatcaacaaatacaatcatgacattgccctgctggaactggatgagcctctggtgctgaacagctacgtcactccaatctgcattgctgacaaagagtataccaatatcttcctgaagtttggatcagggtacgtgagcggctggggaagagtcttccacaagggcaggagcgccctggtgctccagtatctgcgagtgcctctggtcgatcgagctacctgtctgaggtctaccaagtttacaatctacaacaacatgttctgcgctgggtttcacgagggaggacgagactcctgtcagggcgattctgggggcccacatgtgacagaggtcgaaggcaccagcttcctgactggcatcatttcctggggagaggaatgtgcaatgaagggaaaatacgggatctacaccaaagtgagccgctatgtgaactggatcaaggaaaaaaccaaactgacctaatga

SEQ ID NO: 10 - protein, 28 aa, synthetic construct
  REGION 1..28: note = FIX Signal Peptide
MQRVNMIMAESPGLITICLLGYLLSAEC

SEQ ID NO: 11 - protein, 18 aa, synthetic construct
  REGION 1..18: note = FIX Propeptide
TVFLDHENANKILNRPKR

SEQ ID NO: 12 - protein, 415 aa, synthetic construct
  REGION 1..415: note = FIX R338L
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 13 - skipped

SEQ ID NO: 14 - protein, 323 aa, synthetic construct
  REGION 1..323: note = CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 15 - protein, 11 aa, synthetic construct
  REGION 1..11: note = albumin binding peptide core sequence
DICLPRWGCLW

SEQ ID NO: 16 - protein, 5 aa, synthetic construct
  REGION 1..5: note = peptide linker
GGGGS

SEQ ID NO: 17 - protein, 10 aa, synthetic construct
  REGION 1..10: note = peptide linker
GGGGSGGGGS

SEQ ID NO: 18 - protein, 32 aa, synthetic construct
  REGION 1..32: note = CTP peptide
DPRFQDSSSSKAPPPSLPSPSRLPGPSDTPIL

SEQ ID NO: 19 - protein, 28 aa, synthetic construct
  REGION 1..28: note = CTP PEPTIDE
SSSSKAPPPSLPSPSRLPGPSDTPILPQ

SEQ ID NO: 20 - protein, 20 aa, synthetic construct
  REGION 1..20: note = PAS peptide
ASPAAPAPASPAAPAPSAPA

SEQ ID NO: 21 - protein, 20 aa, synthetic construct
  REGION 1..20: note = PAS peptide
AAPASPAPAAPSAPAPAAPS

SEQ ID NO: 22 - protein, 20 aa, synthetic construct
  REGION 1..20: note = PAS Peptide
APSSPSPSAPSSPSPASPSS

SEQ ID NO: 23 - protein, 19 aa, synthetic construct
  REGION 1..19: note = PAS Peptide
APSSPSPSAPSSPSPASPS

SEQ ID NO: 24 - protein, 20 aa, synthetic construct
  REGION 1..20: note = PAS Peptide
SSPSAPSPSSPASPSPSSPA

SEQ ID NO: 25 - protein, 24 aa, synthetic construct
  REGION 1..24: note = Pas Peptide
AASPAAPSAPPAAASPAAPSAPPA

SEQ ID NO: 26 - protein, 20 aa, synthetic construct
  REGION 1..20: note = Pas Peptide
ASAAAPAAASAAASAPSAAA

SEQ ID NO: 27 - DNA, 23 bp, synthetic construct
  misc_feature 1..23: note = miR142 Target
  source 1..23: mol_type = other DNA
tccataaagtaggaaacactaca

(19) United States
(12) Patent Application Publication
(10) Pub. No.: US 2026/0209799 A1
(43) Pub. Date: Jul. 23, 2026

(54) USE OF LENTIVIRAL VECTORS EXPRESSING FACTOR IX

(71) Applicant: BIOVERATIV THERAPEUTICS INC., Waltham, MA (US)

(72) Inventors: Tongyao LIU, Waltham, MA (US); Susannah PATARROYO-WHITE, Waltham, MA (US); Douglas DRAGER, Waltham, MA (US); Alessio CANTORE, Milan (IT); Luigi NALDINI, Milan (IT)

(21) Appl. No.: 19/349,047

(22) Filed: Oct. 3, 2025

Related U.S. Application Data
(63) Continuation of application No. 16/704,400, filed on Dec. 5, 2019, now abandoned.
(60) Provisional application No. 62/776,393, filed on Dec. 6, 2018.

(51) Int. Cl. C12N 15/86; A61P 7/04; C07K 14/745

(52) U.S. Cl. CPC C12N 15/86; A61P 7/04; C07K 14/745; C12N 2750/14143

(57) ABSTRACT

The present disclosure provides lentiviral vectors comprising a nucleic acid sequence encoding a polypeptide with factor IX (FIX) activity, and methods of using such lentiviral vectors. The liver-targeted lentiviral vectors disclosed herein can be used for gene therapy, wherein the lentiviral gene delivery enables stable integration of the transgene expression cassette into the genome of targeted cells (e.g., hepatocytes) of pediatric (e.g., neonatal) or adult subjects, achieving an improvement in FIX expression at low lentiviral vector doses. The present disclosure also provides methods of treating bleeding disorders such as hemophilia (e.g., hemophilia B) comprising administering to a subject in need thereof a liver-targeted lentiviral vector comprising a nucleic acid sequence encoding a polypeptide with FIX activity sequence at low dosages.

### RELATED APPLICATIONS

This application is a continuation of U.S. patent application Ser. No. 16/704,400, filed Dec. 5, 2019, which claims the benefit of priority to U.S. Provisional Patent Application Ser. No. 62/776,393, filed Dec. 6, 2018, the entire disclosures of which are hereby incorporated herein by reference.

### REFERENCE TO SEQUENCE LISTING SUBMITTED ELECTRONICALLY

The instant application contains a Sequence Listing which has been submitted electronically in XML format and is hereby incorporated by reference in its entirety. Said XML file, created on Sep. 29, 2025, is named 770261_SA9-468CON_ST26.xml and is 46,680 bytes in size.

### BACKGROUND OF THE DISCLOSURE

The blood coagulation pathway, in part, involves the formation of an enzymatic complex of Factor VIIIa (FVIIIa) and Factor IXa (FIXa) (Xase complex) on the surface of platelets. FIXa is a serine protease with relatively weak catalytic activity without its cofactor FVIIIa. The Xase complex cleaves Factor X (FX) into Factor Xa (FXa), which in turn interacts with Factor Va (FVa) to cleave prothrombin and generate thrombin. Hemophilia B is a bleeding disorder caused by mutations and/or deletions in the FIX gene resulting in a deficiency of FIX activity.

In hemophilia, blood clotting is disturbed by a lack of certain plasma blood clotting factors. Hemophilia B (also known as Christmas disease) is one of the most common inherited bleeding disorders in the world. It is caused by a deficiency in Factor IX that may result from either the decreased synthesis of the Factor IX protein or a defective molecule with reduced activity. It results in decreased in vivo and in vitro blood clotting activity and requires extensive medical monitoring throughout the life of the affected individual. Without effective prophylaxis, recurrent haemarthroses lead to the development of progressive and disabling arthropathy and poor quality of life (Giangrande P., Expert Opin Pharmacother. 2005; 6:1517-24).

Treatment of hemophilia B occurs by replacement of the missing clotting factor by exogenous factor concentrates highly enriched in Factor IX. However, generating such a concentrate from blood is fraught with technical difficulties. Therefore, there exists a need in the art for a FIX therapy that overcomes the difficulties and limitations of current replacement therapies. Gene therapy stands as a potential approach for lasting treatment of hemophilia B, by the stable integration of a transgene expression cassette comprising a nucleic acid sequence encoding a polypeptide with FIX activity into the genome of targeted cells.

### SUMMARY OF THE DISCLOSURE

The present disclosure provides methods of preventing or treating hemophilia in a subject in need thereof comprising administering to the subject an effective dose of a lentiviral vector comprising a nucleotide sequence encoding a polypeptide with factor IX (FIX) activity, wherein the lentiviral vector is packaged in CD47 overexpressing HEK293T cells that comprise a higher level of surface CD47 protein expression than a control lentiviral vector produced in unmodified HEK293T cells (ATCC® CRL-11268™), and wherein the effective dose is reduced relative to a control dose of the control lentiviral vector necessary to induce the same FIX activity as the lentiviral vector.

In some embodiments, the control lentiviral vector comprises 19 molecules/μm² of CD47 on the surface of the control lentiviral vector. In some embodiments, the lentiviral vector comprises at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold more CD47 protein on the surface of the lentiviral vector than the control lentiviral vector produced in HEK293T cells (ATCC® CRL-11268™).

In some embodiments, the effective dose is less than about 5×10¹⁰ transducing units/kg (TU/kg), less than 4×10¹⁰ TU/kg, less than 3×10¹⁰ TU/kg, less than 2×10¹⁰ TU/kg, less than 1×10¹⁰ TU/kg, less than 9×10⁹ TU/kg, less than 8×10⁹ TU/kg, less than 7×10⁹ TU/kg, less than 6×10⁹ TU/kg, less than 5×10⁹ TU/kg, less than 4×10⁹ TU/kg, less than 3×10⁹ TU/kg, less than 2×10⁹ TU/kg less than 1×10⁹ TU/kg, less than about 9×10⁸ TU/kg, or less than about 8×10⁸ TU/kg.

In some embodiments, the subject exhibits one or more of the following properties following the administration: (a) a decreased macrophage transduction of the lentiviral vector relative to the control lentiviral vector; (b) a reduced allo-specific immune response to the lentiviral vector relative to the control lentiviral vector; (c) a FIX activity of at least 30%, relative to normal FIX activity at least 3 weeks after administration; (d) a tissue specific expression of the lentiviral vector in the liver, spleen, or both the liver and the spleen; and (e) any combination of (a)-(d).

In some embodiments, the allo-specific immune response comprises the release of a cytokine in response to the lentiviral vector. In some embodiments, the cytokine is selected from the group consisting of MIP-1a, MIP-1b, MCP-1, and any combination thereof. In some embodiments, the subject exhibits a lower level of MIP-1a expression following the administration of the lentiviral vector relative to the expression of MIP-1a following administration of the control lentiviral vector. In some embodiments, the subject exhibits a lower level of MIP-1b expression following the administration of the lentiviral vector relative to the expression of MIP-1b following administration of the control lentiviral vector. In some embodiments, the subject exhibits a lower level of MCP-1 expression following the administration of the lentiviral vector relative to the expression of MCP-1 following administration of the control lentiviral vector.

In some embodiments, the subject exhibits FIX activity of at least about 75%, at least about 100%, at least about 125%, at least about 150%, at least about 175%, at least about 200%, at least about 225%, at least about 250%, at least about 275%, or at least about 300%, relative to normal FIX activity, at least three weeks after administration of the lentiviral vector. In some embodiments, the subject exhibits FIX activity of at least about 150%, relative to normal FIX activity, at least three weeks after administration of the lentiviral vector. In some embodiments, plasma FIX activity at 24 hours to 48 hours post administration of the lentiviral vector is increased relative to a subject administered the control dose of the control lentiviral vector. In some embodiments, the plasma FIX activity is increased after the administration by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold, relative to a subject administered the control dose of the control lentiviral vector.

In some embodiments, the subject exhibits increased localization of the lentiviral vector to the liver, spleen, or both the liver and the spleen following administration of the lentiviral vector, relative to an organ other than the liver and spleen in the subject. In some embodiments, the increased localization is characterized by a vector copy number (VCN) of the lentiviral vector that is at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold higher in the liver, spleen, or both the liver and the spleen following administration of the lentiviral vector, relative to an organ other than the liver and spleen in the subject. In some embodiments, the increased localization is characterized by a VCN of the lentiviral vector that is at least 10-fold higher in the liver, spleen, or both the liver and the spleen following administration of the lentiviral vector, relative to an organ other than the liver and spleen in the subject. In some embodiments, the increased localization is characterized by a VCN of the lentiviral vector that is at least 50-fold higher in the liver, spleen, or both the liver and the spleen following administration of the lentiviral vector, relative to an organ other than the liver and spleen in the subject. In some embodiments, the increased localization is characterized by a VCN of the lentiviral vector that is at least 100-fold higher in the liver, spleen or both the liver and the spleen following administration of the lentiviral vector, relative to an organ other than the liver and spleen in the subject.

In some embodiments, the CD47 is a human CD47. In some embodiments, the human CD47 comprises an amino acid sequence at least 60%, at least about 70%, at least 70%, at least about 80%, at least 85%, at least about 90%, at least 95%, at least about 96%, at least 97%, at least about 98%, at least 99%, or about 100% identical to the amino acid sequence set forth in SEQ ID NO: 14. In some embodiments, the lentiviral vector does not comprise an MHC-I polypeptide. In some embodiments, the lentiviral vector is produced in a host cell expressing a high concentration of the CD47 compared to the HEK293T cells (ATCC® CRL-11268™).

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

The present disclosure also provides methods of preventing or treating hemophilia in a subject in need thereof comprising administering to the subject less than 5×10¹⁰ transducing units/kg (TU/kg) of a lentiviral vector comprising a nucleotide sequence encoding a polypeptide with factor IX (FIX) activity, wherein the lentiviral vector comprises a nucleotide sequence having at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7. In some embodiments, the nucleotide sequence has at least 85% sequence identity to the nucleotide sequence set forth in SEQ ID NO: 1. In some embodiments, the nucleotide sequence has at least 85% sequence identity to nucleotides 139-1386 of the nucleotide sequence set forth in SEQ ID NO: 2. In some embodiments, the nucleotide sequence has at least 85% sequence identity to nucleotides 139-1386 of the nucleotide sequence setforth in SEQ ID NO: 3. In some embodiments, the nucleotide sequence has at least 85% sequence identity to nucleotides 139-1386 of the nucleotide sequence set forth in SEQ ID NO: 4. In some embodiments, the nucleotide sequence has at least 85% sequence identity to nucleotides 139-1386 of the nucleotide sequence set forth in SEQ ID NO: 5. In some embodiments, the nucleotide sequence has at least 85% sequence identity to nucleotides 139-1386 of the nucleotide sequence set forth in SEQ ID NO: 6. In some embodiments, the nucleotide sequence has at least 85% sequence identity to nucleotides 139-1386 of the nucleotide sequence set forth in SEQ ID NO: 7.

In some embodiments, the dose is about 5×10¹⁰ TU/kg, about 4.5×10¹⁰ TU/kg, about 4×10¹⁰ TU/kg, about 3.5×10¹⁰ TU/kg, about 3×10¹⁰ TU/kg, about 2.5×10¹⁰ TU/kg, about 2×10¹⁰ TU/kg, about 1.5×10¹⁰ TU/kg, about 1×10¹⁰ TU/kg, about 9.5×10⁹ TU/kg, about 9×10⁹ TU/kg, about 8.5×10⁹ TU/kg, about 8×10⁹ TU/kg, about 7.5×10⁹ TU/kg, about 7×10⁹ TU/kg, about 6.5×10⁹ TU/kg, about 6×10⁹ TU/kg, about 5.5×10⁹ TU/kg, about 5×10⁹ TU/kg, about 4.5×10⁹ TU/kg, about 4×10⁹ TU/kg, about 3.5×10⁹ TU/kg, about 3×10⁹ TU/kg, about 2.5×10⁹ TU/kg, about 2×10⁹ TU/kg, about 1.5×10⁹ TU/kg, about 1×10⁹ TU/kg, about 9.5×10⁸ TU/kg, about 9×10⁸ TU/kg, about 8.5×10⁸ TU/kg, about 8×10⁸ TU/kg, about 7.5×10⁸ TU/kg, about 7×10⁸ TU/kg, about 6.5×10⁸ TU/kg, about 6×10⁸ TU/kg, about 5.5×10⁸ TU/kg, about 5×10⁸ TU/kg, about 4.5×10⁸ TU/kg, about 4×10⁸ TU/kg, about 3.5×10⁸ TU/kg, about 3×10⁸ TU/kg, about 2.5×10⁸ TU/kg, about 2×10⁸ TU/kg, about 1.5×10⁸ TU/kg, or about 1×10⁸ TU/kg. In some embodiments, the dose is less than 5×10¹⁰ TU/kg, less than 4.5×10¹⁰ TU/kg, less than 4×10¹⁰ TU/kg, less than 3.5×10¹⁰ TU/kg, less than 3×10¹⁰ TU/kg, less than 2.5×10¹⁰ TU/kg, less than 2×10¹⁰ TU/kg, less than 1.5×10¹⁰ TU/kg, less than 1×10¹⁰ TU/kg, less than 9.5×10⁹ TU/kg, less than 9×10⁹ TU/kg, less than 8.5×10⁹ TU/kg, less than 8×10⁹ TU/kg, less than 7.5×10⁹ TU/kg, less than 7×10⁹ TU/kg, less than 6.5×10⁹ TU/kg, less than 6×10⁹ TU/kg, less than 5.5×10⁹ TU/kg, less than 5×10⁹ TU/kg, less than 4.5×10⁹ TU/kg, less than 4×10⁹ TU/kg, less than 3.5×10⁹ TU/kg, less than 3×10⁹ TU/kg, less than 2.5×10⁹ TU/kg, less than 2×10⁹ TU/kg, less than 1.5×10⁹ TU/kg, less than 1×10⁹ TU/kg, less than about 9.5×10⁸ TU/kg, less than about 9×10⁸ TU/kg, less than about 8.5×10⁸ TU/kg, less than about 8×10⁸ TU/kg, less than about 7.5×10⁸ TU/kg, less than about 7×10⁸ TU/kg, less than about 6.5×10⁸ TU/kg, less than about 6×10⁸ TU/kg, less than about 5.5×10⁸ TU/kg, less than about 5×10⁸ TU/kg, less than about 4.5×10⁸ TU/kg, less than about 4×10⁸ TU/kg, less than about 3.5×10⁸ TU/kg, less than about 3×10⁸ TU/kg, less than about 2.5×10⁸ TU/kg, less than about 2×10⁸ TU/kg, less than about 1.5×10⁸ TU/kg, or less than about 1×10⁸ TU/kg. In some embodiments, the dose is between 1×10⁸ and 5×10¹⁰ TU/kg, between 1×10⁸ and 5×10⁹ TU/kg, between 1×10⁸ and 1×10⁹ TU/kg, between 1×10⁸ and 1×10¹⁰ TU/kg, between 1×10⁹ and 5×10¹⁰ TU/kg, between 2×10⁹ and 5×10¹⁰ TU/kg, between 3×10⁹ and 5×10¹⁰ TU/kg, between 4×10⁹ and 5×10¹⁰ TU/kg, between 5×10⁹ and 5×10¹⁰ TU/kg, between 1×10⁹ and 6×10⁹ TU/kg, between 2×10⁹ and 6×10⁹ TU/kg, between 3×10⁹ and 6×10⁹ TU/kg, between 4×10⁹ and 6×10⁹ TU/kg, between 5×10⁹ and 6×10⁹ TU/kg, between 6×10⁹ and 5×10¹⁰ TU/kg, between 7×10⁹ and 5×10¹⁰ TU/kg, 8×10⁹ and 5×10¹⁰ TU/kg, between 9×10⁹ and 5×10¹⁰ TU/kg, between 10¹⁰ and 5×10¹⁰ TU/kg, between 1.5×10¹⁰ and 5×10¹⁰ TU/kg, between 2×10¹⁰ and 5×10¹⁰ TU/kg, between 2.5×10¹⁰ and 5×10¹⁰ TU/kg, between 3×10¹⁰ and 5×10¹⁰ TU/kg, between 3.5×10¹⁰ and 5×10¹⁰ TU/kg, between 4×10¹⁰ and 5×10¹⁰ TU/kg, or between 4.5×10¹⁰ and 5×10¹⁰ TU/kg. In some embodiments, the dose is between 1×10⁹ and 5×10¹⁰ TU/kg, between 1×10⁹ and 4.5×10¹⁰ TU/kg, between 1×10⁹ and 4×10¹⁰ TU/kg, between 1×10⁹ and 3.5×10¹⁰ TU/kg, between 1×10⁹ and 3×10¹⁰ TU/kg, between 1×10⁹ and 2.5×10¹⁰ TU/kg, between 1×10⁹ and 2×10¹⁰ TU/kg, between 1×10⁹ and 1.5×10¹⁰ TU/kg, between 1×10⁹ and 10¹⁰ TU/kg, between 1×10⁹ and 9×10⁹ TU/kg, between 1×10⁹ and 8×10⁹ TU/kg, between 1×10⁹ and 7×10⁹ TU/kg, between 1×10⁹ and 6×10⁹ TU/kg, between 1×10⁹ and 5×10⁹ TU/kg, between 1×10⁹ and 4×10⁹ TU/kg, between 1×10⁹ and 3×10⁹ TU/kg, and between 1×10⁹ and 2×10⁹ TU/kg. In some embodiments, the dose is between 1×10¹⁰ and 2×10¹⁰ TU/kg, between 1.1×10¹⁰ and 1.9×10¹⁰ TU/kg, between 1.2×10¹⁰ and 1.8×10¹⁰ TU/kg, between 1.3×10¹⁰ and 1.7×10¹⁰ TU/kg, or between 1.4×10¹⁰ and 1.6×10¹⁰ TU/kg. In some embodiments, the dose is about 4×10⁹ TU/kg to about 6×10⁹ TU/kg.

In some embodiments, the lentiviral vector is administered as a single dose or multiple doses. In some embodiments, the lentiviral vector is administered via intravenous injection. In some embodiments, the subject is a pediatric subject. In some embodiments, the subject is an adult subject. In some embodiments, the polypeptide with FIX activity comprises an amino acid sequence having at least 90% sequence identity to the amino acid sequence set forth in SEQ ID NO: 12. In some embodiments, the polypeptide with FIX activity comprises the amino acid sequence set forth in SEQ ID NO: 12.

In some embodiments, the lentiviral vector comprises a tissue specific promoter. In some embodiments, the tissue specific promoter selectively enhances expression of the polypeptide with FIX activity in a target liver cell. In some embodiments, the tissue specific promoter that selectively enhances expression of the polypeptide with FIX activity in a target liver cell comprises an APOA2 promoter, SERPINA1 (hAAT) promoter, mTTR promoter, MIR122 promoter, or any combination thereof. In some embodiments, the target liver cell is a hepatocyte. In some embodiments, the isolated nucleic acid molecule is stably integrated into the genome of the hepatocyte.

In some embodiments, the lentiviral vector comprises a splice donor site. In some embodiments, the lentiviral vector comprises a splice acceptor site. In some embodiments, the lentiviral vector comprises a gag sequence, a pol sequence, a rev sequence, a rev responsive element (RRE), or any combination thereof. In some embodiments, the gag sequence is a full-length or truncated gag sequence. In some embodiments, the lentiviral vector comprises an enhancer, a target sequence for a microRNA, a post-transcriptional regulatory element, a packaging signal, a poly-A sequence, an intron sequence, or any combination thereof.

In some embodiments, the dose of the lentiviral vector is administered at once or divided into at least two sub-doses. In some embodiments, the dose of lentiviral vector is repeated at least twice.

In some embodiments, the nucleotide sequence encoding a polypeptide with FIX activity further comprises a nucleic acid sequence encoding a signal peptide. In some embodiments, the nucleic acid sequence encoding a signal peptide has at least 60%, at least 70%, at least 80%, at least 90%, at least 95%, at least 96%, at least 97%, at least 98%, at least 99%, or 100% sequence identity to: (i) nucleotides 1-84 of SEQ ID NO: 2; (ii) nucleotides 1-84 of SEQ ID NO: 3; (iii) nucleotides 1-84 of SEQ ID NO: 4; (iv) nucleotides 1-84 of SEQ ID NO: 5; (v) nucleotides 1-84 of SEQ ID NO: 6; or (vi) nucleotides 1-84 of SEQ ID NO: 7. In some embodiments, the nucleotide sequence encoding a polypeptide with FIX activity further comprises a nucleic acid sequence encoding a propeptide. In some embodiments, the nucleic acid sequence encoding a propeptide has at least 60%, at least 70%, at least 80%, at least 90%, at least 95%, at least 96%, at least 97%, at least 98%, at least 99%, or 100% sequence identity to: (i) nucleotides 85-138 of SEQ ID NO: 2; (ii) nucleotides 85-138 of SEQ ID NO: 3; (iii) nucleotides 85-138 of SEQ ID NO: 4; (iv) nucleotides 85-138 of SEQ ID NO: 5; (v) nucleotides 85-138 of SEQ ID NO: 6; or (vi) nucleotides 85-138 of SEQ ID NO: 7.

In some embodiments, the nucleotide sequence encoding a polypeptide with FIX activity further comprises a heterologous nucleotide sequence encoding a heterologous amino acid sequence. In some embodiments, the heterologous amino acid sequence is an albumin, an immunoglobulin Fc region, an XTEN sequence, the C-terminal peptide (CTP) of the β subunit of human chorionic gonadotropin, a PAS sequence, a HAP sequence, a CTP peptide sequence, a transferrin, albumin-binding moiety, or any fragments, derivatives, variants, or combinations of these polypeptides. In some embodiments, the heterologous amino acid sequence is linked to the N-terminus or the C-terminus of the amino acid sequence encoded by a nucleotide sequence encoding a polypeptide with FIX activity or inserted between two amino acids in the amino acid sequence. In some embodiments, the heterologous moiety is inserted within the polypeptide with FIX activity immediately downstream of an amino acid corresponding to of amino acid 103 of SEQ ID NO: 2, amino acid 105 of SEQ ID NO: 2, amino acid 142 of SEQ ID NO: 2, amino acid 149 of SEQ ID NO: 2, amino acid 162 of SEQ ID NO: 2, amino acid 166 of SEQ ID NO: 2, amino acid 174 of SEQ ID NO: 2, amino acid 224 of SEQ ID NO: 2, amino acid 226 of SEQ ID NO: 2, amino acid 228 of SEQ ID NO: 2, amino acid 413 of SEQ ID NO: 2, or any combination thereof. In some embodiments, the FIX polypeptide is a R338L variant FIX polypeptide.

In some embodiments, the lentiviral vector is produced in a host cell. In some embodiments, the host cell expresses CD47. In some embodiments, the host cell is modified to overexpress CD47. In some embodiments, the host cell does not express MHC-I. In some embodiments, the host cell is CD47^{high}/MHC-I⁻. In some embodiments, the host cell is a CD47^{high}/MHC-1⁻ HEK 293T cell.

The present disclosure also provides lentiviral vectors comprising a nucleotide sequence comprising (i) a tissue specific promoter, and (ii) a nucleic acid sequence as set forth in SEQ ID NO: 1, wherein the tissue specific promoter drives expression of the nucleic acid sequence in a liver cell.

The present disclosure also provides lentiviral vectors comprising a nucleotide sequence comprising (i) a splice donor site; (ii) a splice acceptor site; (iii) a gag sequence; (iv) a Rev responsive element; (v) an enhancer; (vi) a post-transcriptional regulatory element, (vii) a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to sequence identity to the nucleotide sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7, and (viii) a target sequence for a microRNA.

In some embodiments, the nucleic acid sequence encodes a polypeptide with FIX activity, which comprises an amino acid sequence having at least 90% sequence identity to the amino acid sequence set forth in SEQ ID NO: 12. In some embodiments, the polypeptide with FIX activity comprises the amino acid sequence set forth in SEQ ID NO: 12.

In some embodiments, the surface of the lentiviral vector comprises a higher level of CD47 protein than a control lentiviral vector produced in HEK293T cells (ATCC® CRL-11268™). In some embodiments, the surface of the lentiviral vector does not comprise MHC-I.

The present disclosure is also directed to methods of treating hemophilia in a subject in need thereof, comprising administering to the subject an effective dose of a lentiviral vector disclosed herein. In some embodiments, the effective dose is less than about 5×10¹⁰ transducing units/kg (TU/kg), less than 4×10¹⁰ TU/kg, less than 3×10¹⁰ TU/kg, less than 2×10¹⁰ TU/kg, less than 1×10¹⁰ TU/kg, less than 9×10⁹ TU/kg, less than 8×10⁹ TU/kg, less than 7×10⁹ TU/kg, less than 6×10⁹ TU/kg, less than 5×10⁹ TU/kg, less than 4×10⁹ TU/kg, less than 3×10⁹ TU/kg, less than 2×10⁹ TU/kg less than 1×10⁹ TU/kg, less than about 9×10⁸ TU/kg, or less than about 8×10⁸ TU/kg. In some embodiments, the effective dose is about 5×10¹⁰ TU/kg, about 4.5×10¹⁰ TU/kg, about 4×10¹⁰ TU/kg, about 3.5×10¹⁰ TU/kg, about 3×10¹⁰ TU/kg, about 2.5×10¹⁰ TU/kg, about 2×10¹⁰ TU/kg, about 1.5×10¹⁰ TU/kg, about 1×10¹⁰ TU/kg, about 9.5×10⁹ TU/kg, about 9×10⁹ TU/kg, about 8.5×10⁹ TU/kg, about 8×10⁹ TU/kg, about 7.5×10⁹ TU/kg, about 7×10⁹ TU/kg, about 6.5×10⁹ TU/kg, about 6×10⁹ TU/kg, about 5.5×10⁹ TU/kg, about 5×10⁹ TU/kg, about 4.5×10⁹ TU/kg, about 4×10⁹ TU/kg, about 3.5×10⁹ TU/kg, about 3×10⁹ TU/kg, about 2.5×10⁹ TU/kg, about 2×10⁹ TU/kg, about 1.5×10⁹ TU/kg, about 1×10⁹ TU/kg, about 9.5×10⁸ TU/kg, about 9×10⁸ TU/kg, about 8.5×10⁸ TU/kg, about 8×10⁸ TU/kg, about 7.5×10⁸ TU/kg, about 7×10⁸ TU/kg, about 6.5×10⁸ TU/kg, about 6×10⁸ TU/kg, about 5.5×10⁸ TU/kg, about 5×10⁸ TU/kg, about 4.5×10⁸ TU/kg, about 4×10⁸ TU/kg, about 3.5×10⁸ TU/kg, about 3×10⁸ TU/kg, about 2.5×10⁸ TU/kg, about 2×10⁸ TU/kg, about 1.5×10⁸ TU/kg, or about 1×10⁸ TU/kg. In some embodiments, the effective dose is less than 5×10¹⁰ TU/kg, less than 4.5×10¹⁰ TU/kg, less than 4×10¹⁰ TU/kg, less than 3.5×10¹⁰ TU/kg, less than 3×10¹⁰ TU/kg, less than 2.5×10¹⁰ TU/kg, less than 2×10¹⁰ TU/kg, less than 1.5×10¹⁰ TU/kg, less than 1×10¹⁰ TU/kg, less than 9.5×10⁹ TU/kg, less than 9×10⁹ TU/kg, less than 8.5×10⁹ TU/kg, less than 8×10⁹ TU/kg, less than 7.5×10⁹ TU/kg, less than 7×10⁹ TU/kg, less than 6.5×10⁹ TU/kg, less than 6×10⁹ TU/kg, less than 5.5×10⁹ TU/kg, less than 5×10⁹ TU/kg, less than 4.5×10⁹ TU/kg, less than 4×10⁹ TU/kg, less than 3.5×10⁹ TU/kg, less than 3×10⁹ TU/kg, less than 2.5×10⁹ TU/kg, less than 2×10⁹ TU/kg, less than 1.5×10⁹ TU/kg, less than 1×10⁹ TU/kg, less than about 9.5×10⁸ TU/kg, less than about 9×10⁸ TU/kg, less than about 8.5×10⁸ TU/kg, less than about 8×10⁸ TU/kg, less than about 7.5×10⁸ TU/kg, less than about 7×10⁸ TU/kg, less than about 6.5×10⁸ TU/kg, less than about 6×10⁸ TU/kg, less than about 5.5×10⁸ TU/kg, less than about 5×10⁸ TU/kg, less than about 4.5×10⁸ TU/kg, less than about 4×10⁸ TU/kg, less than about 3.5×10⁸ TU/kg, less than about 3×10⁸ TU/kg, less than about 2.5×10⁸ TU/kg, less than about 2×10⁸ TU/kg, less than about 1.5×10⁸ TU/kg, or less than about 1×10⁸ TU/kg. In some embodiments, the effective dose is between 1×10⁸ and 5×10¹⁰ TU/kg, between 1×10⁸ and 5×10⁹ TU/kg, between 1×10⁸ and 1×10⁹ TU/kg, between 1×10⁸ and 1×10¹⁰ TU/kg, between 1×10⁹ and 5×10¹⁰ TU/kg, between 2×10⁹ and 5×10¹⁰ TU/kg, between 3×10⁹ and 5×10¹⁰ TU/kg, between 4×10⁹ and 5×10¹⁰ TU/kg, between 5×10⁹ and 5×10¹⁰ TU/kg, between 1×10⁹ and 6×10⁹ TU/kg, between 2×10⁹ and 6×10⁹ TU/kg, between 3×10⁹ and 6×10⁹ TU/kg, between 4×10⁹ and 6×10⁹ TU/kg, between 5×10⁹ and 6×10⁹ TU/kg, between 6×10⁹ and 5×10¹⁰ TU/kg, between 7×10⁹ and 5×10¹⁰ TU/kg, 8×10⁹ and 5×10¹⁰ TU/kg, between 9×10⁹ and 5×10¹⁰ TU/kg, between 10¹⁰ and 5×10¹⁰ TU/kg, between 1.5×10¹⁰ and 5×10¹⁰ TU/kg, between 2×10¹⁰ and 5×10¹⁰ TU/kg, between 2.5×10¹⁰ and 5×10¹⁰ TU/kg, between 3×10¹⁰ and 5×10¹⁰ TU/kg, between 3.5×10¹⁰ and 5×10¹⁰ TU/kg, between 4×10¹⁰ and 5×10¹⁰ TU/kg, or between 4.5×10¹⁰ and 5×10¹⁰ TU/kg. In some embodiments, the effective dose is between 1×10⁹ and 5×10¹⁰ TU/kg, between 1×10⁹ and 4.5×10¹⁰ TU/kg, between 1×10⁹ and 4×10¹⁰ TU/kg, between 1×10⁹ and 3.5×10¹⁰ TU/kg, between 1×10⁹ and 3×10¹⁰ TU/kg, between 1×10⁹ and 2.5×10¹⁰ TU/kg, between 1×10⁹ and 2×10¹⁰ TU/kg, between 1×10⁹ and 1.5×10¹⁰ TU/kg, between 1×10⁹ and 10¹⁰ TU/kg, between 1×10⁹ and 9×10⁹ TU/kg, between 1×10⁹ and 8×10⁹ TU/kg, between 1×10⁹ and 7×10⁹ TU/kg, between 1×10⁹ and 6×10⁹ TU/kg, between 1×10⁹ and 5×10⁹ TU/kg, between 1×10⁹ and 4×10⁹ TU/kg, between 1×10⁹ and 3×10⁹ TU/kg, and between 1×10⁹ and 2×10⁹ TU/kg. In some embodiments, the effective dose is between 1×10¹⁰ and 2×10¹⁰ TU/kg, between 1.1×10¹⁰ and 1.9×10¹⁰ TU/kg, between 1.2×10¹⁰ and 1.8×10¹⁰ TU/kg, between 1.3×10¹⁰ and 1.7×10¹⁰ TU/kg, or between 1.4×10¹⁰ and 1.6×10¹⁰ TU/kg. In some embodiments, the effective dose is about 4×10⁹ TU/kg to about 6×10⁹ TU/kg.

In some embodiments, the lentiviral vector is administered as a single dose or multiple doses. In some embodiments, the lentiviral vector is administered via intravenous injection. In some embodiments, the subject is a pediatric subject. In some embodiments, the subject is an adult subject.

In some embodiments, the nucleotide sequence as set forth in SEQ ID NO: 1. The present disclosure is also directed to vectors comprising a nucleic acid sequence disclosed herein. In some embodiments, the vector comprises a tissue specific promoter. In some embodiments, the tissue specific promoter selectively enhances expression of the polypeptide with FIX activity in a target liver cell. In some embodiments, the tissue specific promoter that selectively enhances expression of the polypeptide with FIX activity in a target liver cell comprises an APOA2 promoter, SERPINA1 (hAAT) promoter, mTTR promoter, MIR122 promoter, or any combination thereof. In some embodiments, the target liver cell is a hepatocyte.

In some embodiments, the vector comprises a splice donor site. In some embodiments, the vector comprises a splice acceptor site. In some embodiments, the vector comprises a gag sequence, a pol sequence, a rev sequence, a rev responsive element (RRE), or any combination thereof. In some embodiments, the gag sequence is a full-length or truncated gag sequence. In some embodiments, the vector comprises an enhancer, a target sequence for a microRNA, a post-transcriptional regulatory element, a packaging signal, a poly-A sequence, an intron sequence, or any combination thereof.

The present disclosure is also directed to a cell comprising a nucleic acid sequence or a vector disclosed herein. In some embodiments, the cell is a mammalian cell. In some embodiments, the cell is a CHO cell, a HEK293 cell, a BHK21 cell, a PER.C6® cell, a NS0 cell, and a CAP cell. In some embodiments, the cell is a human cell. In some embodiments, the cell expresses a CD47 protein. In some embodiments, the cell is modified to overexpress CD47. In some embodiments, the cell comprises at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold more CD47 protein on the surface of the cell as compared to a control cell that is not modified to overexpress CD47. In some embodiments, the CD47 is a human CD47. In some embodiments, the cell does not express MHC-I.

The present disclosure is also directed to methods of producing a lentiviral vector comprising culturing a cell disclosed herein under suitable conditions.

### BRIEF DESCRIPTION OF THE DRAWINGS

FIG. 1 is a vector map of a lentiviral vector (“LV-coFIX-1-R338L”) comprising a nucleic acid encoding a polypeptide with FIX activity.

FIGS. 2A-2B are graphical representations of the plasma FIX activity for HemB mice following administration at eight weeks of age with a lentiviral vector comprising a nucleic acid sequence encoding a polypeptide with FIX activity. FIG. 2A shows plasma FIX activity administered LV-coFIX-1-R338L via tail vein injection at a dose of 3E9, 7.5E9, 2E10, or 6E10 TU/kg, and FIG. 2B shows the corresponding dose response curve. Error bars represent standard deviation (FIG. 2B).

FIGS. 3A-3B are graphical representations of the plasma FIX activity (FIG. 3A) and plasma FIX antigen (FIG. 3B) at various time points up to 6 months for HemB mice administered at 8 weeks of age with LV-coFIX-1-R338L via tail vein injection at a dose of 7.5E9 (circles), 2E10 (squares), or 6E10 (triangles) TU/kg. Error bars represent standard deviation (FIGS. 3A-3B).

FIGS. 4A-4B is a graphical representation of persistent FIX expression at various time points up to 6 months (FIG. 4A) and LV-FIX dose response with similar doses (FIG. 4B) at neonatal, adolescent, or adult stages. HemB mice were administered eight-weeks of age (squares) or two-days of age (circles) with LV-coFIX-1-R338L intravenous injection at a dose of 7.5E9, 2E10, or 6E10 TU/kg; or HemB mice were administered two-weeks of age (triangles) with LV-coFIX-1-R338L intravenous injection at a dose of 3E9, 7.5E9, or 2E10 TU/kg, as indicated (FIG. 4A). Dose response was measured by FIX activity at the various doses tested in FIG. 4A in HemB mice at eight-weeks of age (squares), two-weeks of age (triangles), and two-days of age (circles). Error bars represent standard deviation (FIGS. 4A-4B).

FIG. 5 is a plot illustrating the vector copy number (VCN) of lentiviral vector in NOD mice macrophages following administration of a control lentiviral vector (LV; black circles) or lentiviral vector having high surface levels of CD47 (CD47 hi LV; grey circles), and the number of lentiviral vector molecules present in macrophages was determined. HEK293T cell VCN data are shown as a control. Error bars represent standard deviation.

FIGS. 6A-6C are graphical representations of the plasma FIX activity (FIG. 6A), plasma FIX antigen (FIG. 6B), and FIX function (represented by APTT time; FIG. 6C) in Macaca nemestrina monkeys following administration of a lentiviral vector comprising a nucleic acid sequence encoding a polypeptide with FIX activity packaged in a control lentiviral vector (LV-FIX; #1, #2, and #3), a lentiviral vector having high surface levels of CD47 (CD47 hi LV-FIX; #4, #5, and #6), or a vehicle control (#7).

FIGS. 7A-7B are graphical representations of steady state lentiviral vector-mediated FIX expression in Macaca nemestrina monkeys following administration of CD47^{high} lentiviral vector comprising a lentiviral vector comprising a nucleic acid sequence encoding a polypeptide with FIX activity, administered at a dose of E9 TU/kg, as represented by plasma FIX activity (FIG. 7A) and plasma FIX antigen (FIX. 7B). Error bars represent standard deviation (FIGS. 7A-7B).

FIGS. 8A-8D are graphical representations of ALT levels (FIG. 8A), AST levels (FIG. 8B), lympho levels (FIG. 8C), and body temperature (FIG. 8D) in Macaca nemestrina monkeys following administration of vehicle (white circles), control lentiviral vector (LV; black circles) or lentiviral vector having high surface levels of CD47 (CD47 hi LV; grey circles) lentiviral vector comprising a lentiviral vector comprising a nucleic acid sequence encoding a polypeptide with FIX activity.

FIGS. 9A-9C are graphical representations of the expression levels of MIP-1a (9A), MIP-1b (9B), and MCP-1 (9C) in Macaca nemestrina monkeys following administration of Vehicle (black circles), control lentiviral vector (LV; black squares) or lentiviral vector having high surface levels of CD47 (CD47 hi LV; open squares) lentiviral vector comprising a lentiviral vector comprising a nucleic acid sequence encoding a polypeptide with FIX activity.

FIG. 10 is a scatter plot illustrating the tissue specific distribution (represented by VCN) of lentiviral vectors comprising a nucleic acid sequence encoding a polypeptide with FIX activity following administration to Macaca nemestrina monkeys, vehicle (triangle), control lentiviral vector (revised triangle) or CD47 hi LV; circles). Each data set represents an individual Macaca nemestrina monkey.

FIGS. 11A and 11B are graphical representations of steady state lentiviral vector-mediated FIX expression in Macaca nemestrina monkeys following administration of CD47^{high} lentiviral vector comprising a lentiviral vector comprising a nucleic acid sequence encoding a polypeptide with FIX activity, administered at a dose of 2.5 E9 TU/kg, as represented by plasma FIX activity (FIG. 11A) and plasma FIX antigen (FIG. 11B).

### DETAILED DESCRIPTION OF THE DISCLOSURE

The present disclosure describes lentiviral vectors comprising a nucleic acid encoding a polypeptide with FIX activity, and methods of using the same. Accordingly, in some aspects, the present disclosure is directed to gene therapy comprising the administration of lentiviral vectors comprising nucleic acid molecules comprising nucleic acid sequences encoding polypeptides with FIX activity. In particular aspects, the present disclosure is directed to methods of treating bleeding disorders such as hemophilia (e.g., hemophilia B) comprising administering to the subject a lentiviral vector comprising a codon optimized FIX nucleic acid sequence targeted to the liver (e.g., to hepatocytes). The present disclosure meets an important need in the art through a gene therapy approach that results in the stable integration of a transgene expression cassette comprising a nucleic acid sequence encoding a polypeptide with FIX activity into the genome of the targeted cells.

This system demonstrates increased long-term expression of polypeptides having FIX activity in the targeted cells (e.g., hepatocytes), when the lentiviral vector is administering to the subject at least one dose of 5×10¹⁰ transducing units/kg (TU/kg) or lower, e.g., about 1.5×10¹⁰ TU/kg or less, or about 1.5×10⁹ TU/kg or less, or about 1×10⁸ TU/kg or less.

The Exemplary constructs of the disclosure are illustrated in the accompanying Figures and sequence listing.

In order to provide a clear understanding of the specification and claims, the following definitions are provided below.

### I. Definitions

It is to be noted that the term “a” or “an” entity refers to one or more of that entity: for example, “a nucleotide sequence” is understood to represent one or more nucleotide sequences. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “isolated” for the purposes of the present disclosure designates a biological material (cell, polypeptide, polynucleotide, or a fragment, variant, or derivative thereof) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.” No particular level of purification is required. Recombinantly produced polypeptides and proteins expressed in host cells are considered isolated for the purpose of the disclosure, as are native or recombinant polypeptides which have been separated, fractionated, or partially or substantially purified by any suitable technique.

“Nucleic acids,” “nucleic acid molecules,” “oligonucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then, that a single vector can contain just a single coding region, or comprise two or more coding regions.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide or a functional derivative of that sequence that retains the ability to direct the secretion of the polypeptide that is operably associated with it. Alternatively, a heterologous mammalian signal peptide, e.g., a human tissue plasminogen activator (TPA) or mouse ß-glucuronidase signal peptide, or a functional derivative thereof, can be used.

The term “polypeptide having FIX activity,” as used herein, refers to a polypeptide that has one or more activity associated with clotting factor IX. A number of tests are available to assess the function of the coagulation system, including FIX: activated partial thromboplastin time (aPTT) test, chromogenic assay, ROTEM® assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM©), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FIX. It is used in conjunction with prothrombin time (PT), which measures the extrinsic pathway.

ROTEM® analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

A polynucleotide which encodes a gene product, e.g., a polypeptide, can include a promoter and/or other expression (e.g., transcription or translation) control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage. The term “yield,” as used herein, refers to the amount of a polypeptide produced by the expression of a gene.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like. Selectable marker genes can also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” refers to a “replicon,” which is a unit length of a nucleic acid that replicates sequentially and which comprises an origin of replication, such as a plasmid, phage or cosmid, to which another nucleic acid segment can be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter. “Lentiviral vector,” as used herein refers to a replication-defective hybrid viral particle. In some contexts, the lentiviral vector refers to the lentiviral vector particle and the enclosed lentivirus genome. In some contexts, the lentiviral vector refers to the lentivirus genome, including any modifications thereof.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (lie or 1): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the disclosure and include norleucine, omithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g., M, F, W, S, Y, N, Q, C). These amino acids can participate in hydrophobic interactions and electrostatic interactions. As used herein, the term “charged amino acid” includes amino acids that can have non-zero net charge on their side chains (e.g., R, K, H, E, D). These amino acids can participate in hydrophobic interactions and electrostatic interactions.

Also included in the present disclosure are fragments or variants of polypeptides, and any combination thereof. The term “fragment” or “variant” when referring to polypeptide binding domains or binding molecules of the present disclosure include any polypeptides which retain at least some of the properties (e.g., FcRn binding affinity for an FcRn binding domain or Fc variant, coagulation activity for polypeptide having FIX activity) of the reference polypeptide. Fragments of polypeptides include proteolytic fragments, as well as deletion fragments, in addition to specific antibody fragments discussed elsewhere herein, but do not include the naturally occurring full-length polypeptide (or mature polypeptide). Variants of polypeptide binding domains or binding molecules of the present disclosure include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case can be, as determined by the match between strings of such sequences. “Identity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity are codified in publicly available computer programs. Sequence alignments and percent identity calculations can be performed using sequence analysis software such as the Megalign program of the LASERGENE bioinformatics computing suite (DNASTAR Inc., Madison, WI), the GCG suite of programs (Wisconsin Package Version 9.0, Genetics Computer Group (GCG), Madison, WI), BLASTP, BLASTN, BLASTX (Altschul et al., J. Mol. Biol. 215:403 (1990)), and DNASTAR (DNASTAR, Inc. 1228 S. Park St. Madison, WI 53715 USA).

Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

A “fusion” or “chimeric” protein comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a FIX domain of the disclosure with an Ig Fc domain. A fusion protein is created, for example, by chemical synthesis, or by creating and translating a polynucleotide in which the peptide regions are encoded in the desired relationship. A chimeric protein can further comprise a second amino acid sequence associated with the first amino acid sequence by a covalent, non-peptide bond or a non-covalent bond.

As used herein, the term “insertion site” refers to a position in a polypeptide having a FIX activity, or fragment, variant, or derivative thereof, which is immediately upstream of the position at which a heterologous moiety can be inserted. An “insertion site” is specified as a number, the number being the number of the amino acid in human FIX R338L variant (SEQ ID NOs: 11-12) to which the insertion site corresponds, which is immediately N-terminal to the position of the insertion, unless otherwise indicated.

The phrase “immediately downstream of an amino acid” as used herein refers to position right next to the terminal carboxyl group of the amino acid. Similarly, the phrase “immediately upstream of an amino acid” refers to the position right next to the terminal amine group of the amino acid.

The terms “inserted,” “is inserted,” “inserted into” or grammatically related terms, as used herein refers to the position of a heterologous moiety in a recombinant FIX polypeptide, relative to the analogous position in native mature human FIX. As used herein the terms refer to the characteristics of the recombinant FIX polypeptide relative to native mature human FIX, and do not indicate, imply or infer any methods or process by which the recombinant FIX polypeptide was made.

As used herein, the term “half-life” refers to a biological half-life of a particular polypeptide in vivo. Half-life can be represented by the time required for half the quantity administered to a subject to be cleared from the circulation and/or other tissues in the animal. When a clearance curve of a given polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the polypeptide in the intravascular space. In some embodiments, FIX and chimeric proteins comprising FIX are monophasic, and thus do not have an alpha phase, but just the single beta phase. Therefore, in certain embodiments, the term half-life as used herein refers to the half-life of the polypeptide in the β-phase.

The term “linked” as used herein refers to a first amino acid sequence or nucleotide sequence covalently or non-covalently joined to a second amino acid sequence or nucleotide sequence, respectively. The first amino acid or nucleotide sequence can be directly joined or juxtaposed to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

As used herein the term “associated with” refers to a covalent or non-covalent bond formed between a first amino acid chain and a second amino acid chain. In one embodiment, the term “associated with” means a covalent, non-peptide bond or a non-covalent bond. This association can be indicated by a colon, i.e., (:). In another embodiment, it means a covalent bond except a peptide bond. For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, an hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

The term “monomer-dimer hybrid” used herein refers to a chimeric protein comprising a first polypeptide chain and a second polypeptide chain, which are associated with each other by a disulfide bond, wherein the first chain comprises a clotting factor, e.g., FIX, and a first Fc region and the second chain comprises, consists essentially of, or consists of a second Fc region without the clotting factor. The monomer-dimer hybrid construct thus is a hybrid comprising a monomer aspect having only one clotting factor and a dimer aspect having two Fc regions.

Hemostasis, as used herein, means the stopping or slowing of bleeding or hemorrhage; or the stopping or slowing of blood flow through a blood vessel or body part.

Hemostatic disorder, as used herein, means a genetically inherited or acquired condition characterized by a tendency to hemorrhage, either spontaneously or as a result of trauma, due to an impaired ability or inability to form a fibrin clot. Examples of such disorders include the hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for vWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this can increase bleeding risk.

The lentiviral vectors comprising the isolated nucleic acid molecule of the disclosure can be used prophylactically. As used herein the term “prophylactic treatment” refers to the administration of a molecule prior to a bleeding episode. In one embodiment, the subject in need of a general hemostatic agent is undergoing, or is about to undergo, surgery. For example, a lentiviral vector of the disclosure can be administered prior to or after surgery as a prophylactic. The lentiviral vector of the disclosure can be administered during or after surgery to control an acute bleeding episode. The surgery can include, but is not limited to, liver transplantation, liver resection, dental procedures, or stem cell transplantation.

The lentiviral vectors of the disclosure are also used for on-demand treatment. The term “on-demand treatment” refers to the administration of a lentiviral vector disclosed herein in response to symptoms of a bleeding episode or before an activity that can cause bleeding. In one aspect, the on-demand treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject can have trauma, uremia, a hereditary bleeding disorder (e.g., FIX deficiency) a platelet disorder, or resistance owing to the development of antibodies to clotting factors.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one embodiment, the term “treating” or “treatment” means maintaining a FIX trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering a lentiviral vector of the disclosure. In another embodiment, treating or treatment means maintaining a FIX trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FIX activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FIX activity in a non-hemophiliac subject. In one embodiment, the term “treating” or “treatment” means maintaining a FIX trough level at least about 30 IU/dL, 40 IU/dL, 50 IU/dL, 60 IU/dL, 70 IU/dL, 80 IU/dL, 90 IU/dL, 100 IU/dL, 110 IU/dL, 120 IU/dL, 130 IU/dL, 140 IU/dL, or 150 IU/dL in a subject by administering a lentiviral vector of the disclosure. In another embodiment, treating or treatment means maintaining a FIX trough level between about 10 and about 20 IU/dL, about 20 and about 23 IU/dL, about 30 and about 40 IU/dL, about 40 and about 50 IU/dL, about 50 and about 60 IU/dL, about 60 and about 70 IU/dL, about 70 and about 80 IU/dL, about 80 and about 90 IU/dL, about 90 and about 100 IU/dL, about 110 and about 120 IU/dL, about 120 and about 130 IU/dL, about 130 and about 140 IU/dL, or about 140 and about 150 IU/dL. Treatment or treating of a disease or condition can also include maintaining FIX activity in a subject at a level comparable to at least about 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145% or 150% of the FIX activity in a non-hemophiliac subject. The minimum trough level required for treatment can be measured by one or more known methods and can be adjusted (increased or decreased) for each person.

“Administering,” as used herein, means to give a pharmaceutically acceptable FIX-encoding nucleic acid molecule, FIX polypeptide, or vector comprising a FIX-encoding nucleic acid molecule of the disclosure to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. The nucleic acid molecules, polypeptides, and vectors can be administered as part of a pharmaceutical composition comprising at least one excipient.

As used herein, the phrase “subject in need thereof” includes subjects, such as mammalian subjects, that would benefit from administration of a nucleic acid molecule, a polypeptide, or vector of the disclosure, e.g., to improve hemostasis. In one embodiment, the subjects include, but are not limited to, individuals with hemophilia. In another embodiment, the subjects include, but are not limited to, the individuals who have developed a FIX inhibitor and thus are in need of a bypass therapy. The subject can be an adult or a minor (e.g., under 12 years old). In some embodiments, the subject is a female. In other embodiments, the subject is a male.

As used herein, the term “clotting factor,” refers to molecules, or analogs thereof, naturally occurring or recombinantly produced which prevent or decrease the duration of a bleeding episode in a subject. In other words, it means molecules having pro-clotting activity, i.e., are responsible for the conversion of fibrinogen into a mesh of insoluble fibrin causing the blood to coagulate or clot. An “activatable clotting factor” is a clotting factor in an inactive form (e.g., in its zymogen form) that is capable of being converted to an active form.

Clotting activity, as used herein, means the ability to participate in a cascade of biochemical reactions that culminates in the formation of a fibrin clot and/or reduces the severity, duration or frequency of hemorrhage or bleeding episode.

As used herein the terms “heterologous” or “exogenous” refer to such molecules that are not normally found in a given context, e.g., in a cell or in a polypeptide. For example, an exogenous or heterologous molecule can be introduced into a cell and are only present after manipulation of the cell, e.g., by transfection or other forms of genetic engineering or a heterologous amino acid sequence can be present in a protein in which it is not naturally found.

As used herein, the term “heterologous nucleotide sequence” refers to a nucleotide sequence that does not naturally occur with a given polynucleotide sequence. In one embodiment, the heterologous nucleotide sequence encodes a polypeptide capable of extending the half-life of FIX. In another embodiment, the heterologous nucleotide sequence encodes a polypeptide that increases the hydrodynamic radius of FIX. In other embodiments, the heterologous nucleotide sequence encodes a polypeptide that improves one or more pharmacokinetic properties of FIX without significantly affecting its biological activity or function (e.g., its procoagulant activity). In some embodiments, FIX is linked or connected to the polypeptide encoded by the heterologous nucleotide sequence by a linker. Non-limiting examples of polypeptide moieties encoded by heterologous nucleotide sequences include an immunoglobulin constant region or a portion thereof, albumin or a fragment thereof, an albumin-binding moiety, a transferrin, the PAS polypeptides of U.S. Pat Application No. 20100292130, a HAP sequence, transferrin or a fragment thereof, the C-terminal peptide (CTP) of the β subunit of human chorionic gonadotropin, albumin-binding small molecule, an XTEN sequence, FcRn binding moieties (e.g., complete Fc regions or portions thereof which bind to FcRn), single chain Fc regions (ScFc regions, e.g., as described in US 2008/0260738, WO 2008/012543, or WO 2008/1439545), polyglycine linkers, polyserine linkers, peptides and short polypeptides of 6-40 amino acids of two types of amino acids selected from glycine (G), alanine (A), serine (S), threonine (T), glutamate (E) and proline (P) with varying degrees of secondary structure from less than 50% to greater than 50%, amongst others, or two or more combinations thereof. In some embodiments, the polypeptide encoded by the heterologous nucleotide sequence is linked to a non-polypeptide moiety. Non-limiting examples of the non-polypeptide moieties include polyethylene glycol (PEG), albumin-binding small molecules, polysialic acid (PAS), hydroxyethyl starch (HES), a derivative thereof, or any combinations thereof.

As used herein, the term “Fc region” is defined as the portion of a polypeptide which corresponds to the Fc region of native Ig, i.e., as formed by the dimeric association of the respective Fc domains of its two heavy chains. A native Fc region forms a homodimer with another Fc region. In contrast, the term “genetically-fused Fc region” or “single-chain Fc region” (scFc region), as used herein, refers to a synthetic dimeric Fc region comprised of Fc domains genetically linked within a single polypeptide chain (i.e., encoded in a single contiguous genetic sequence).

In one embodiment, the “Fc region” refers to the portion of a single Ig heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an Ig constant region, depending on the Ig isotype can include the CH2, CH3, and CH4 domains, as well as the hinge region. Chimeric proteins comprising an Fc region of an Ig bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

As used herein, the term “optimized,” with regard to nucleotide sequences, refers to a polynucleotide sequence that encodes a polypeptide, wherein the polynucleotide sequence has been mutated to enhance a property of that polynucleotide sequence. In some embodiments, the optimization is done to increase transcription levels, increase translation levels, increase steady-state mRNA levels, increase or decrease the binding of regulatory proteins such as general transcription factors, increase or decrease splicing, or increase the yield of the polypeptide produced by the polynucleotide sequence. Examples of changes that can be made to a polynucleotide sequence to optimize it include codon optimization, G/C content optimization, removal of repeat sequences, removal of AT rich elements, removal of cryptic splice sites, removal of cis-acting elements that repress transcription or translation, adding or removing poly-T or poly-A sequences, adding sequences around the transcription start site that enhance transcription, such as Kozak consensus sequences, removal of sequences that could form stem loop structures, removal of destabilizing sequences, and two or more combinations thereof.

### II. FIX Lentiviral Gene Therapy

Somatic gene therapy has been explored as a possible treatment for bleeding disorders, and in particular, hemophilia. Gene therapy is a particularly appealing treatment for hemophilia because of its potential to cure the disease through continuous endogenous production of FIX following a single administration of a vector encoding FIX. Hemophilia B is well suited for a gene replacement approach because its clinical manifestations are attributable to decreased expression of functional FIX.

Lentiviral vectors are gaining prominence as gene delivery vehicles due to their large capacity and ability to sustain transgene expression via integration. Lentiviral vectors have been evaluated in numerous ex-vivo cell therapy clinical programs with promising efficacy and safety profiles.

The present disclosure provides methods of preventing or treating hemophilia in a subject in need thereof comprising administering to the subject an effective dose of a lentiviral vector comprising a nucleotide sequence encoding a polypeptide with FIX activity. In some embodiments, the lentiviral vector is packaged in a lentiviral particle that comprises a higher level of surface CD47 protein expression than a control lentiviral vector, e.g., a control lentiviral vector produced in HEK293 cells (ATCC® CRL-1573™) without the high level of surface CD47 protein expression, i.e., normal (naturally-occurring) level of surface CD47 protein expression. In some embodiments, the effective dose is reduced relative to a control dose of the control lentiviral vector necessary to induce the same FIX activity as the lentiviral vector.

Other aspects of the disclosure provide methods of preventing or treating hemophilia in a subject in need thereof comprising administering to the subject less than 5×10¹⁰ transducing units/kg (TU/kg) of a lentiviral vector comprising a nucleotide sequence encoding a polypeptide with factor IX (FIX) activity, wherein the lentiviral vector comprises a nucleotide sequence having at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

In some embodiments, the subject exhibits a decreased macrophage transduction of the lentiviral vector following the administration, relative to the control lentiviral vector. In some embodiments, the subject exhibits a reduced allo-specific immune response to the lentiviral vector following the administration, relative to the control lentiviral vector. In some embodiments, the subject exhibits a FIX activity of at least 30%, relative to normal FIX activity, at least 3 weeks after administration. In some embodiments, the subject exhibits a tissue specific expression of the lentiviral vector in the liver, spleen, or both the liver and the spleen following the administration.

In some embodiments, the allo-specific response comprises the release of a cytokine in response to the administered lentiviral vector. In some embodiments, the subject exhibits a lower expression of a cytokine associated with an allo-specific response following the administration of the lentiviral vector relative to the expression of the cytokine following administration of the control lentiviral vector. In some embodiments, the cytokine is a pro-inflammatory cytokine. In certain embodiments, the cytokine is selected from the group consisting of MIP-1a, MIP-1b, MCP-1, interleukin-2 (IL-2), interferon gamma, and any combination thereof. In certain embodiments, the subject exhibits a lower level of MIP-1a expression following the administration of the lentiviral vector relative to the expression of MIP-1a following administration of the control lentiviral vector. In certain embodiments, the subject exhibits a lower level of MIP-1b expression following the administration of the lentiviral vector relative to the expression of MIP-1b following administration of the control lentiviral vector. In certain embodiments, the subject exhibits a lower level of MCP-1 expression following the administration of the lentiviral vector relative to the expression of MCP-1 following administration of the control lentiviral vector. In certain embodiments, the subject exhibits a lower level of IL-2 expression following the administration of the lentiviral vector relative to the expression of IL-2 following administration of the control lentiviral vector. In certain embodiments, the subject exhibits a lower level of interferon gamma expression following the administration of the lentiviral vector relative to the expression of interferon gamma following administration of the control lentiviral vector.

In some embodiments, the expression of the cytokine is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, relative to the expression of the cytokine following administration of the control lentiviral vector. In certain embodiments, the expression of the cytokine is undetectable following administration of the lentiviral vector. In certain embodiments, the subject has undetectable expression of a cytokine selected from the group consisting of MIP-1a, MIP-1b, MCP-1, interleukin-2 (IL-2), interferon gamma, and any combination thereof following administration of the lentiviral vector.

In some embodiments, the subject exhibits increased plasma FIX activity after administration of the lentiviral vector relative to the plasma FIX activity before the administration. In some embodiments, the increase is observed at least 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 3 weeks, at least about 4 weeks, at least about 5 weeks, at least about 6 weeks, at least about 7 weeks, or at least about 8 weeks following the administration. In some embodiments, the plasma FIX activity at about 12 hours to about 60 hours, about 12 hours to about 48 hours, about 12 hours to about 36 hours, about 12 hours to about 24 hours, about 24 hours to about 60 hours, about 24 hours to about 48 hours, about 24 hours to about 36 hours, about 36 hours to about 60 hours, about 36 hours to about 48 hours, or about 48 hours to about 60 hours post administration of the lentiviral vector is increased relative to a subject administered the control dose of the control lentiviral vector. In some embodiments, the FIX activity following the administration of the lentiviral vector is at least about 75%, at least about 100%, at least about 125%, at least about 150%, at least about 175%, at least about 200%, at least about 225%, at least about 250%, at least about 275%, or at least about 300%, relative to normal FIX activity, at least one week, two weeks, or three weeks after administration of the lentiviral vector. In certain embodiments, the subject exhibits FIX activity of at least about 150%, relative to normal FIX activity, at least three weeks after administration of the lentiviral vector. In certain embodiments, the subject exhibits FIX activity of at least about 200%, relative to normal FIX activity, at least three weeks after administration of the lentiviral vector. In certain embodiments, the subject exhibits FIX activity of at least about 225%, relative to normal FIX activity, at least three weeks after administration of the lentiviral vector.

In some embodiments, the plasma FIX activity is increased after the administration by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold, relative to a subject administered the control dose of the control lentiviral vector.

In some embodiments, following the administration, the lentiviral vector is specifically localized to the liver, the spleen, or both the liver and spleen of the subject, wherein a greater concentration of the lentiviral vector is found in the liver, the spleen, or both the liver and the spleen than another organ in the body of the subject. In some embodiments, the other organ in the body of the subject is selected from the group consisting of a testis, a lymph-node, a muscle, the thymus, a kidney, a lung, the heart, the frontal cortex, the thalamus, the caudate nucleus, the colliculi, the cerebellum, a peripheral blood mononuclear cell (PBMC), and any combination thereof. In some embodiments, the subject exhibits increased localization of the lentiviral vector to the liver, spleen, or both the liver and the spleen following administration of the lentiviral vector, relative to an organ other than the liver and spleen in the subject. The localization of the lentiviral vector can be measured and/or expressed using any methods and/or units known in the art. In some embodiments, the increased localization is characterized by a vector copy number (VCN) of the lentiviral vector that is at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, at least about 200-fold, at least about 250-fold, at least about 300-fold, at least about 400-fold, or at least about 500-fold higher in the liver, spleen, or both the liver and the spleen following administration of the lentiviral vector, relative to an organ other than the liver and spleen in the subject.

In certain embodiments, the subject exhibits increased localization of the lentiviral vector to the liver, the spleen, or both the liver and the spleen, wherein the increased localization is characterized by a VCN of the lentiviral vector that is at least 10-fold higher in the liver, spleen, or both the liver and the spleen following administration of the lentiviral vector, relative to an organ other than the liver and spleen in the subject. In certain embodiments, the subject exhibits increased localization of the lentiviral vector to the liver, the spleen, or both the liver and the spleen, wherein the increased localization is characterized by a VCN of the lentiviral vector that is at least 50-fold higher in the liver, spleen, or both the liver and the spleen following administration of the lentiviral vector, relative to an organ other than the liver and spleen in the subject. In certain embodiments, the subject exhibits increased localization of the lentiviral vector to the liver, the spleen, or both the liver and the spleen, wherein the increased localization is characterized by a VCN of the lentiviral vector that is at least 100-fold higher in the liver, spleen, or both the liver and the spleen following administration of the lentiviral vector, relative to an organ other than the liver and spleen in the subject. In certain embodiments, the subject exhibits increased localization of the lentiviral vector to the liver, the spleen, or both the liver and the spleen, wherein the increased localization is characterized by a VCN of the lentiviral vector that is at least 150-fold higher in the liver, spleen, or both the liver and the spleen following administration of the lentiviral vector, relative to an organ other than the liver and spleen in the subject.

### A. Immune Response Inhibition

In some embodiments, the lentiviral vector, e.g., the lentiviral particle, contains one or more polypeptides on its surface that inhibit an immune response to the lentiviral vector following administration to a human subject. Certain aspects of the present disclosure are directed to lentiviral vectors or methods of administering lentiviral vectors to a subject, wherein the lentiviral vector contains CD47 on its surface. In some embodiments, the surface of the lentiviral vector comprises one or more CD47 molecules. CD47 is a “marker of self” protein, which is ubiquitously expressed on human cells. Surface expression of CD47 inhibits macrophage-induced phagocytosis of endogenous cells through the interaction of CD47 and macrophage expressed-SIRPα. Cells expressing high levels of CD47 are less likely to be targeted and destroyed by human macrophages in vivo.

In some embodiments, the lentiviral vector comprises a high concentration of CD47 polypeptide molecules on its surface. In some embodiments, the lentiviral vector comprises a heterologous polynucleotide encoding a CD47 protein, wherein the heterologous polynucleotide encoding the CD47 is expressed. In some embodiments, the lentiviral vector further comprises a heterologous polynucleotide encoding a CD47 protein, wherein the heterologous polynucleotide encoding the CD47 is expressed.

In some embodiments, the lentiviral vector has a higher level of a CD47 protein because it is produced in a cell line that has a high expression level of CD47. In certain embodiments, the lentiviral vector is produced in a CD47^{high} cell, wherein the cell has high expression of CD47 on the cell membrane. In particular embodiments, the lentiviral vector is produced in a CD47^{high} HEK 293T cell, wherein the HEK 293T is modified to have increased expression of CD47 relative to the expression of CD47 in unmodified HEK293T cells. In some embodiments, the HEK 293T cell is modified to overexpress endogenous CD47 relative to unmodified HEK 293T cells. In some embodiments, the HEK 293T cell is modified by transducing the HEK293T cell with a heterologous CD47-expressing vector. In some embodiments, the heterologous CD47-expressing vector comprises a retroviral vector. In certain embodiments, the retroviral vector is a γ-retroviral vector. In some embodiments, the heterologous CD47-expressing vector is not capable of being cross packaged by the lentiviral vector.

In certain aspects, the present disclosure is directed to methods of preventing or treating hemophilia in a subject in need thereof comprising administering to the subject an effective dose of a lentiviral vector comprising a nucleotide sequence encoding a polypeptide with FIX activity, wherein the lentiviral vector comprises a higher level of surface CD47 protein expression than control lentiviral vectors produced in HEK293 cells (ATCC® CRL-1573™) and wherein the effective dose is reduced relative to a control dose of the control lentiviral vector necessary to induce the same FIX activity as the lentiviral vector. Without being bound by any mechanism, CD47 expression on the surface of the lentiviral vector is believed to protect the lentiviral vector form degradation and/or removal by the subject's immune system and to prevent and/or reduce the immune response to the lentiviral vector.

In some embodiments, the CD47 is human CD47 (NCBI Accession No. NP_001768.1). In certain embodiments, the CD47 comprises an amino acid sequence at least 60%, at least about 70%, at least 70%, at least about 80%, at least 85%, at least about 90%, at least 95%, at least about 96%, at least 97%, at least about 98%, at least 99%, or about 100% identical to the amino acid sequence set forth in SEQ ID NO: 14. In particular embodiments, the human CD47 comprises the amino acid sequence set forth in SEQ ID NO: 14.

In some embodiments, the CD47 is expressed by the lentiviral vector. In other embodiments, the CD47 is not expressed by the lentiviral vector. In certain embodiments, the lentiviral vector is expressed in a host cell, wherein the host cell is modified to express CD47. In some embodiments, the host cell is modified to overexpress CD47. In certain embodiments, the lentiviral vector is produced in a host cell expressing a high concentration of CD47 compared to the HEK293 cells (ATCC® CRL-1573™). In particular embodiments, the lentiviral vector is produced in HEK293T cells modified to over express CD47 relative to unmodified HEK293T cells.

In some embodiments, the lentiviral vector comprises a higher level of surface CD47 protein expression than a control lentiviral vector. In certain embodiments, the control lentiviral vector is produced in HEK293 cells (ATCC® CRL-1573™), which is known to produce 19 molecules/μm² (see, e.g., Sosale et al., Methods & Clinical Development 3:16080 (2016) at FIG. S1(d). In some embodiments, the control lentiviral vector comprises less than 20 molecules/μm² of CD47 on the surface of the control lentiviral vector.

In certain embodiments, the lentiviral vector comprises at least about 2-fold to at least about 100-fold, at least about 2-fold to at least about 75-fold, at least about 2-fold to at least about 50-fold, at least about 2-fold to at least about 40-fold, at least about 2-fold to at least about 30-fold, at least about 2-fold to at least about 20-fold, at least about 2-fold to at least about 10-fold, at least about 10-fold to at least about 100-fold, at least about 10-fold to at least about 75-fold, at least about 10-fold to at least about 50-fold, at least about 10-fold to at least about 40-fold, at least about 10-fold to at least about 30-fold, at least about 10-fold to at least about 20-fold, at least about 20-fold to at least about 100-fold, at least about 20-fold to at least about 75-fold, at least about 20-fold to at least about 50-fold, at least about 20-fold to at least about 40-fold, or at least about 20-fold to at least about 30-fold more CD47 protein on the surface of the lentiviral vector than on the surface of the control lentiviral vectors produced in HEK293 cells (ATCC® CRL-1573™). In particular embodiments, the lentiviral vectors comprises at least about 10-fold to at least about 30-fold more CD47 protein on the surface of the lentiviral vector than on the surface of the control lentiviral vector produced in HEK293 cells (ATCC® CRL-1573™). In some embodiments, the lentiviral vector comprises at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10-fold, at least about 11-fold, at least about 12-fold, at least about 13-fold, at least about 14-fold, at least about 15-fold, at least about 20-fold, at least about 25-fold, at least about 30-fold, at least about 35-fold, at least about 40-fold, at least about 45-fold, at least about 50-fold more, at least about 60-fold, at least about 70-fold, at least about 80-fold, at least about 90-fold, or at least about 100-fold more CD47 protein on the surface of the lentiviral vector than on the surface of the control lentiviral vector produced in HEK293 cells (ATCC® CRL-1573™).

In certain embodiments, the lentiviral vector comprises at least about 10-fold more CD47 protein on the surface of the lentiviral vector than on the surface of the control lentiviral vector produced in HEK293 cells (ATCC® CRL-1573™). In certain embodiments, the lentiviral vector comprises at least about 15-fold more CD47 protein on the surface of the lentiviral vector than on the surface of the control lentiviral vector produced in HEK293 cells (ATCC® CRL-1573™). In certain embodiments, the lentiviral vector comprises at least about 20-fold more CD47 protein on the surface of the lentiviral vector than on the surface of the control lentiviral vector produced in HEK293 cells (ATCC® CRL-1573™). In certain embodiments, the lentiviral vector comprises at least about 25-fold more CD47 protein on the surface of the lentiviral vector than on the surface of the control lentiviral vector produced in HEK293 cells (ATCC® CRL-1573™). In certain embodiments, the lentiviral vector comprises at least about 30-fold more CD47 protein on the surface of the lentiviral vector than on the surface of the control lentiviral vector produced in HEK293 cells (ATCC® CRL-1573™).

In some embodiments, the lentiviral vector comprises at least 20 molecules/μm², at least about 25 molecules/μm², at least about 30 molecules/μm², at least about 35 molecules/μm², at least about 40 molecules/μm², at least about 45 molecules/μm², at least about 50 molecules/μm², at least about 55 molecules/μm², at least about 60 molecules/μm², at least about 65 molecules/μm², at least about 70 molecules/μm², at least about 75 molecules/μm², at least about 80 molecules/μm², at least about 85 molecules/μm², at least about 90 molecules/μm², at least about 95 molecules/μm², at least about 100 molecules/μm², at least about 125 molecules/μm², at least about 150 molecules/μm², at least about 175 molecules/μm², at least about 200 molecules/μm² of the CD47 protein on the surface of the lentiviral vector. In some embodiments, the lentiviral vector comprises at least about at least about 225 molecules/μm², at least about 250 molecules/μm², at least about 275 molecules/μm², at least about 300 molecules/μm², at least about 325 molecules/μm², at least about 350 molecules/μm², at least about 375 molecules/μm², at least about 400 molecules/μm², at least about 425 molecules/μm², at least about 450 molecules/μm², at least about 475 molecules/μm², at least about 500 molecules/μm², at least about 525 molecules/μm², at least about 550 molecules/μm², at least about 575 molecules/μm², at least about 600 molecules/μm², at least about 625 molecules/μm², at least about 650 molecules/μm², at least about 675 molecules/μm², at least about 700 molecules/μm², at least about 725 molecules/μm², at least about 750 molecules/μm², at least about 800 molecules/μm², at least about 850 molecules/μm², at least about 900 molecules/μm², at least about 950 molecules/μm², or at least about 1000 molecules/μm² of the CD47 protein on the surface of the lentiviral vector.

In certain embodiments, the lentiviral vector comprises at least about 400 molecules/μm² of the CD47 protein on the surface of the lentiviral vector. In certain embodiments, the lentiviral vector comprises at least about 450 molecules/μm² of the CD47 protein on the surface of the lentiviral vector. In certain embodiments, the lentiviral vector comprises at least about 500 molecules/μm² of the CD47 protein on the surface of the lentiviral vector. In certain embodiments, the lentiviral vector comprises at least about 600 molecules/μm² of the CD47 protein on the surface of the lentiviral vector. In certain embodiments, the lentiviral vector comprises at least about 700 molecules/μm² of the CD47 protein on the surface of the lentiviral vector. In certain embodiments, the lentiviral vector comprises at least about 800 molecules/μm² of the CD47 protein on the surface of the lentiviral vector. In certain embodiments, the lentiviral vector comprises at least about 900 molecules/μm² of the CD47 protein on the surface of the lentiviral vector. In certain embodiments, the lentiviral vector comprises at least about 1000 molecules/μm² of the CD47 protein on the surface of the lentiviral vector.

In some embodiments, the lentiviral vector is expressed in a host cell that is further modified to reduce the immunogenicity of the resulting lentiviral vector. In some embodiments, the lentiviral vector has little or no surface-exposed major histocompatibility complex class I (MHC-1). Surface-exposed MHC-I displays peptide fragments of “non-self” proteins from within a cell, such as protein fragments indicative of an infection, facilitating an immune response against the cell. In some embodiments, the lentiviral vector is produced in a MHC-I^{low} cell, wherein the cell has reduced surface-exposed MHC-I on the cell membrane. In some embodiments, the lentiviral vector is produced in an MHC-I⁻ (or “MHC-I^{free}”, “MHC-1^{neg}” or “MHC-negative”) cell, wherein the cell has no surface-exposed MHC-I.

MHC-I⁻ or MHC-I^{low} cells can be generated using any means known in the art. In some embodiments, MHC-I⁻ or MHC-I^{low} cells are generated by disrupting the expression of one or more gene encoding one or more protein in the MHC. In some embodiments, MHC-I⁻ or MHC-I^{low} cells are generated by disrupting the expression of the gene encoding beta-2-microglobulin (B2M; Ensembl ENSG00000166710; NCBI Protein Accession No. ABB01003). In some embodiments, MHC-I⁻ or MHC-I^{low} cells are generated by permanently disrupting the expression of the gene encoding beta-2-microglobulin (B2M). In some embodiments, MHC-I⁻ or MHC-I^{low} cells are generated by blocking the expression of the gene encoding beta-2-microglobulin (B2M). In some embodiments, MHC-I⁻ or MHC-I^{low} cells are generated by introducing a mutation in the gene encoding beta-2-microglobulin (B2M), wherein the mutation results in the loss of expression of the gene encoding B2M. In some embodiments, MHC-I⁻ or MHC-I^{low} cells are generated by knocking out the gene encoding beta-2-microglobulin (B2M). In certain embodiments, the MHC-I⁻ or MHC-I^{low} cells are generated by modifying HECK 293T cells to block or reduce surface-exposed MHC-I. Unmodified HEK293-T cells have surface-exposed MHC-I. See, e.g., Dellgren et al., PLoS One 10(8):e0135385 (2015).

In some embodiments, the cells are MHC-I⁻ HEK293T cells, wherein the cells have no surface-exposed MHC-I. In certain embodiments, the MHC-I⁻ cells have less than 1% surface exposed MHC-1, relative to unmodified HEK293T cells. In some embodiments, the cells are MHC-I^{low} HEK293T cells, wherein the HEK293T cells are modified to have less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, less than about 1% surface exposed MHC-1, relative to unmodified HEK293T cells. In certain embodiments, the MHC-I^{low} cells have less than 5% surface exposed MHC-1, relative to unmodified HEK293T cells. In certain embodiments, the MHC-I^{low} cells have less than 4% surface exposed MHC-1, relative to unmodified HEK293T cells. In certain embodiments, the MHC-I^{low} cells have less than 3% surface exposed MHC-1, relative to unmodified HEK293T cells. In certain embodiments, the MHC-I^{low} cells have less than 2% surface exposed MHC-1, relative to unmodified HEK293T cells.

In particular embodiments, the lentiviral vector comprises a lipid coat comprising a high concentration of CD47 polypeptides and lacking surface-exposed MHC-I. In certain embodiments, the lentiviral vector is produced in a CD47^{high}/MHC-I^{low} cell line, e.g., a CD47^{high}/MHC-I^{low} HEK 293T cell line. In some embodiments, the lentiviral vector is produced in a CD47^{high}/MHC-I⁻ cell line, e.g., a CD47^{high}/MHC-I⁻ HEK 293T cell line.

In another embodiment, the administration of a lentiviral vector disclosed herein and/or subsequent expression of FIX protein transgene does not induce an immune response in a subject. In some embodiments, the immune response comprises development of antibodies against FIX. In some embodiments, the immune response comprises cytokine secretion. In some embodiments, the immune response comprises activation of B cells, T cells, or both B cells and T cells. In some embodiments, the immune response is an inhibitory immune response, wherein the immune response in the subject reduces the activity of the FIX protein relative to the activity of the FIX in a subject that has not developed an immune response. In certain embodiments, expression of FIX protein by administering the lentiviral vector of the disclosure prevents an inhibitory immune response against the FIX protein or the FIX protein expressed from the isolated nucleic acid molecule or the lentiviral vector.

### B. Dosing

The lentiviral vectors, lentiviral vector particles, and methods of use thereof of the present disclosure allow for the prevention and/or treatment of hemophilia using lower doses of the lentiviral vector than control lentiviral vector produced in than a control lentiviral vector produced in HEK293 cells (ATCC® CRL-1573™). In some embodiments, the lentiviral vectors of the present disclosure are effective at a dose that is reduced relative to a control dose of the control lentiviral vector-necessary to induce the same FIX activity as the lentiviral vector. In some embodiments, the dose of the lentiviral vector is at least about 5%, at least about 10%, at least about 15%, at least about 20%, or at least about 25% that of the control dose of the control lentiviral vector-. In some embodiments, dose is at least about 30%, at least about 35%, at least about 40%, at least about 45%, or at least about 50% that of the control dose of the control lentiviral vector-. In some embodiments, dose is at least about 55%, at least about 60%, at least about 65%, at least about 70%, or at least about 75% that of the control dose of the control lentiviral vector-. In some embodiments, dose is at least about 80%, at least about 85%, at least about 90%, or at least about 95% that of the control dose of the control lentiviral vector-.

In some embodiments, the dose of the lentiviral vector is about 5.0×10¹⁰ TU/kg, about 4.9×10¹⁰ TU/kg, about 4.8×10¹⁰ TU/kg, about 4.7×10¹⁰ TU/kg, about 4.6×10¹⁰ TU/kg, about 4.5×10¹⁰ TU/kg, about 4.4×10¹⁰ TU/kg, about 4.3×10¹⁰ TU/kg, about 4.2×10¹⁰ TU/kg, about 4.1×10¹⁰ TU/kg, about 4.0×10¹⁰ TU/kg, about 3.9×10¹⁰ TU/kg, about 3.8×10¹⁰ TU/kg, about 3.7×10¹⁰ TU/kg, about 3.6×10¹⁰ TU/kg, about 3.5×10¹⁰ TU/kg, about 3.4×10¹⁰ TU/kg, about 3.3×10¹⁰ TU/kg, about 3.2×10¹⁰ TU/kg, about 3.1×10¹⁰ TU/kg, about 3.0×10¹⁰ TU/kg, about 2.9×10¹⁰ TU/kg, about 2.8×10¹⁰ TU/kg, about 2.7×10¹⁰ TU/kg, about 2.6×10¹⁰ TU/kg, about 2.5×10¹⁰ TU/kg, about 2.4×10¹⁰ TU/kg, about 2.3×10¹⁰ TU/kg, about 2.2×10¹⁰ TU/kg, about 2.1×10¹⁰ TU/kg, about 2.0×10¹⁰ TU/kg, about 1.9×10¹⁰ TU/kg, about 1.8×10¹⁰ TU/kg, about 1.7×10¹⁰ TU/kg, about 1.6×10¹⁰ TU/kg, about 1.5×10¹⁰ TU/kg, about 1.4×10¹⁰ TU/kg, about 1.3×10¹⁰ TU/kg, about 1.2×10¹⁰ TU/kg, about 1.1×10¹⁰ TU/kg, or about 1.0×10¹⁰ TU/kg.

In some embodiments, the dose of the lentiviral vector is about 9.9×10⁹ TU/kg, about 9.8×10⁹ TU/kg, about 9.7×10⁹ TU/kg, about 9.6×10⁹ TU/kg, about 9.5×10⁹ TU/kg, about 9.4×10⁹ TU/kg, about 9.3×10⁹ TU/kg, about 9.2×10⁹ TU/kg, about 9.1×10⁹ TU/kg, about 9.0×10⁹ TU/kg, about 8.9×10⁹ TU/kg, about 8.8×10⁹ TU/kg, about 8.7×10⁹ TU/kg, about 8.6×10⁹ TU/kg, about 8.5×10⁹ TU/kg, about 8.4×10⁹ TU/kg, about 8.3×10⁹ TU/kg, about 8.2×10⁹ TU/kg, about 8.1×10⁹ TU/kg, about 8.0×10⁹ TU/kg, about 7.9×10⁹ TU/kg, about 7.8×10⁹ TU/kg, about 7.7×10⁹ TU/kg, about 7.6×10⁹ TU/kg, about 7.5×10⁹ TU/kg, about 7.4×10⁹ TU/kg, about 7.3×10⁹ TU/kg, about 7.2×10⁹ TU/kg, about 7.1×10⁹ TU/kg, about 7.0×10⁹ TU/kg, about 6.9×10⁹ TU/kg, about 6.8×10⁹ TU/kg, about 6.7×10⁹ TU/kg, about 6.6×10⁹ TU/kg, about 6.5×10⁹ TU/kg, about 6.4×10⁹ TU/kg, about 6.3×10⁹ TU/kg, about 6.2×10⁹ TU/kg, about 6.1×10⁹ TU/kg, about 6.0×10⁹ TU/kg, about 5.9×10⁹ TU/kg, about 5.8×10⁹ TU/kg, about 5.7×10⁹ TU/kg, about 5.6×10⁹ TU/kg, about 5.5×10⁹ TU/kg, about 5.4×10⁹ TU/kg, about 5.3×10⁹ TU/kg, about 5.2×10⁹ TU/kg, about 5.1×10⁹ TU/kg, about 5.0×10⁹ TU/kg, about 4.9×10⁹ TU/kg, about 4.8×10⁹ TU/kg, about 4.7×10⁹ TU/kg, about 4.6×10⁹ TU/kg, about 4.5×10⁹ TU/kg, about 4.4×10⁹ TU/kg, about 4.3×10⁹ TU/kg, about 4.2×10⁹ TU/kg, about 4.1×10⁹ TU/kg, about 4.0×10⁹ TU/kg, about 3.9×10⁹ TU/kg, about 3.8×10⁹ TU/kg, about 3.7×10⁹ TU/kg, about 3.6×10⁹ TU/kg, about 3.5×10⁹ TU/kg, about 3.4×10⁹ TU/kg, about 3.3×10⁹ TU/kg, about 3.2×10⁹ TU/kg, about 3.1×10⁹ TU/kg, about 3.0×10⁹ TU/kg, about 2.9×10⁹ TU/kg, about 2.8×10⁹ TU/kg, about 2.7×10⁹ TU/kg, about 2.6×10⁹ TU/kg, about 2.5×10⁹ TU/kg, about 2.4×10⁹ TU/kg, about 2.3×10⁹ TU/kg, about 2.2×10⁹ TU/kg, about 2.1×10⁹ TU/kg, about 2.0×10⁹ TU/kg, about 1.9×10⁹ TU/kg, about 1.8×10⁹ TU/kg, about 1.7×10⁹ TU/kg, about 1.6×10⁹ TU/kg, about 1.5×10⁹ TU/kg, about 1.4×10⁹ TU/kg. about 1.3×10⁹ TU/kg, about 1.2×10⁹ TU/kg, about 1.1×10⁹ TU/kg, or about 1.0×10⁹ TU/kg.

In some embodiments, the dose of the lentiviral vector is about 9.9×10⁸ TU/kg, about 9.8×10⁸ TU/kg, about 9.7×10⁸ TU/kg, about 9.6×10⁸ TU/kg, about 9.5×10⁸ TU/kg, about 9.4×10⁸ TU/kg, about 9.3×10⁸ TU/kg, about 9.2×10⁸ TU/kg, about 9.1×10⁸ TU/kg, about 9.0×10⁸ TU/kg, about 8.9×10⁸ TU/kg, about 8.8×10⁸ TU/kg, about 8.7×10⁸ TU/kg, about 8.6×10⁸ TU/kg, about 8.5×10⁸ TU/kg, about 8.4×10⁸ TU/kg, about 8.3×10⁸ TU/kg, about 8.2×10⁸ TU/kg, about 8.1×10⁸ TU/kg, about 8.0×10⁸ TU/kg, about 7.9×10⁸ TU/kg, about 7.8×10⁸ TU/kg, about 7.7×10⁸ TU/kg, about 7.6×10⁸ TU/kg, about 7.5×10⁸ TU/kg, about 7.4×10⁸ TU/kg, about 7.3×10⁸ TU/kg, about 7.2×10⁸ TU/kg, about 7.1×10⁸ TU/kg, about 7.0×10⁸ TU/kg, about 6.9×10⁸ TU/kg, about 6.8×10⁸ TU/kg, about 6.7×10⁸ TU/kg, about 6.6×10⁸ TU/kg, about 6.5×10⁸ TU/kg, about 6.4×10⁸ TU/kg, about 6.3×10⁸ TU/kg, about 6.2×10⁸ TU/kg, about 6.1×10⁸ TU/kg, about 6.0×10⁸ TU/kg, about 5.9×10⁸ TU/kg, about 5.8×10⁸ TU/kg, about 5.7×10⁸ TU/kg, about 5.6×10⁸ TU/kg, about 5.5×10⁸ TU/kg, about 5.4×10⁸ TU/kg, about 5.3×10⁸ TU/kg, about 5.2×10⁸ TU/kg, about 5.1×10⁸ TU/kg, about 5.0×10⁸ TU/kg, about 4.9×10⁸ TU/kg, about 4.8×10⁸ TU/kg, about 4.7×10⁸ TU/kg, about 4.6×10⁸ TU/kg, about 4.5×10⁸ TU/kg, about 4.4×10⁸ TU/kg, about 4.3×10⁸ TU/kg, about 4.2×10⁸ TU/kg, about 4.1×10⁸ TU/kg, about 4.0×10⁸ TU/kg, about 3.9×10⁸ TU/kg, about 3.8×10⁸ TU/kg, about 3.7×10⁸ TU/kg, about 3.6×10⁸ TU/kg, about 3.5×10⁸ TU/kg, about 3.4×10⁸ TU/kg, about 3.3×10⁸ TU/kg, about 3.2×10⁸ TU/kg, about 3.1×10⁸ TU/kg, about 3.0×10⁸ TU/kg, about 2.9×10⁸ TU/kg, about 2.8×10⁸ TU/kg, about 2.7×10⁸ TU/kg, about 2.6×10⁸ TU/kg, about 2.5×10⁸ TU/kg, about 2.4×10⁸ TU/kg, about 2.3×10⁸ TU/kg, about 2.2×10⁸ TU/kg, about 2.1×10⁸ TU/kg, about 2.0×10⁸ TU/kg, about 1.9×10⁸ TU/kg, about 1.8×10⁸ TU/kg, about 1.7×10⁸ TU/kg, about 1.6×10⁸ TU/kg, about 1.5×10⁸ TU/kg, about 1.4×10⁸ TU/kg. about 1.3×10⁸ TU/kg, about 1.2×10⁸ TU/kg, about 1.1×10³ TU/kg, or about 1.0×10⁸ TU/kg.

In some embodiments, the dose of the lentiviral vector is less than about 5.0×10¹⁰ TU/kg, less than about 4.9×10¹⁰ TU/kg, less than about 4.8×10¹⁰ TU/kg, less than about 4.7×10¹⁰ TU/kg, less than about 4.6×10¹⁰ TU/kg, less than about 4.5×10¹⁰ TU/kg, less than about 4.4×10¹⁰ TU/kg, less than about 4.3×10¹⁰ TU/kg, less than about 4.2×10¹⁰ TU/kg, less than about 4.1×10¹⁰ TU/kg, less than about 4.0×10¹⁰ TU/kg, less than about 3.9×10¹⁰ TU/kg, less than about 3.8×10¹⁰ TU/kg, less than about 3.7×10¹⁰ TU/kg, less than about 3.6×10¹⁰ TU/kg, less than about 3.5×10¹⁰ TU/kg, less than about 3.4×10¹⁰ TU/kg, less than about 3.3×10¹⁰ TU/kg, less than about 3.2×10¹⁰ TU/kg, less than about 3.1×10¹⁰ TU/kg, less than about 3.0×10¹⁰ TU/kg, less than about 2.9×10¹⁰ TU/kg, less than about 2.8×10¹⁰ TU/kg, less than about 2.7×10¹⁰ TU/kg, less than about 2.6×10¹⁰ TU/kg, less than about 2.5×10¹⁰ TU/kg, less than about 2.4×10¹⁰ TU/kg, less than about 2.3×10¹⁰ TU/kg, less than about 2.2×10¹⁰ TU/kg, less than about 2.1×10¹⁰ TU/kg, less than about 2.0×10¹⁰ TU/kg, less than about 1.9×10¹⁰ TU/kg, less than about 1.8×10¹⁰ TU/kg, less than about 1.7×10¹⁰ TU/kg, less than about 1.6×10¹⁰ TU/kg, less than about 1.5×10¹⁰ TU/kg, less than about 1.4×10¹⁰ TU/kg, less than about 1.3×10¹⁰ TU/kg, less than about 1.2×10¹⁰ TU/kg, less than about 1.1×10¹⁰ TU/kg, or less than about 1.0×10¹⁰ TU/kg.

In some embodiments, the dose of the lentiviral vector is less than about 9.9×10⁹ TU/kg, less than about 9.8×10⁹ TU/kg, less than about 9.7×10⁹ TU/kg, less than about 9.6×10⁹ TU/kg, less than about 9.5×10⁹ TU/kg, less than about 9.4×10⁹ TU/kg, less than about 9.3×10⁹ TU/kg, less than about 9.2×10⁹ TU/kg, less than about 9.1×10⁹ TU/kg, less than about 9.0×10⁹ TU/kg, less than about 8.9×10⁹ TU/kg, less than about 8.8×10⁹ TU/kg, less than about 8.7×10⁹ TU/kg, less than about 8.6×10⁹ TU/kg, less than about 8.5×10⁹ TU/kg, less than about 8.4×10⁹ TU/kg, less than about 8.3×10⁹ TU/kg, less than about 8.2×10⁹ TU/kg, less than about 8.1×10⁹ TU/kg, less than about 8.0×10⁹ TU/kg, less than about 7.9×10⁹ TU/kg, less than about 7.8×10⁹ TU/kg, less than about 7.7×10⁹ TU/kg, less than about 7.6×10⁹ TU/kg, less than about 7.5×10⁹ TU/kg, less than about 7.4×10⁹ TU/kg, less than about 7.3×10⁹ TU/kg, less than about 7.2×10⁹ TU/kg, less than about 7.1×10⁹ TU/kg, less than about 7.0×10⁹ TU/kg, less than about 6.9×10⁹ TU/kg, less than about 6.8×10⁹ TU/kg, less than about 6.7×10⁹ TU/kg, less than about 6.6×10⁹ TU/kg, less than about 6.5×10⁹ TU/kg, less than about 6.4×10⁹ TU/kg, less than about 6.3×10⁹ TU/kg, less than about 6.2×10⁹ TU/kg, less than about 6.1×10⁹ TU/kg, less than about 6.0×10⁹ TU/kg, less than about 5.9×10⁹ TU/kg, less than about 5.8×10⁹ TU/kg, less than about 5.7×10⁹ TU/kg, less than about 5.6×10⁹ TU/kg, less than about 5.5×10⁹ TU/kg, less than about 5.4×10⁹ TU/kg, less than about 5.3×10⁹ TU/kg, less than about 5.2×10⁹ TU/kg, less than about 5.1×10⁹ TU/kg, less than about 5.0×10⁹ TU/kg, less than about 4.9×10⁹ TU/kg, less than about 4.8×10⁹ TU/kg, less than about 4.7×10⁹ TU/kg, less than about 4.6×10⁹ TU/kg, less than about 4.5×10⁹ TU/kg, less than about 4.4×10⁹ TU/kg, less than about 4.3×10⁹ TU/kg, less than about 4.2×10⁹ TU/kg, less than about 4.1×10⁹ TU/kg, less than about 4.0×10⁹ TU/kg, less than about 3.9×10⁹ TU/kg, less than about 3.8×10⁹ TU/kg, less than about 3.7×10⁹ TU/kg, less than about 3.6×10⁹ TU/kg, less than about 3.5×10⁹ TU/kg, less than about 3.4×10⁹ TU/kg, less than about 3.3×10⁹ TU/kg, less than about 3.2×10⁹ TU/kg, less than about 3.1×10⁹ TU/kg, less than about 3.0×10⁹ TU/kg, less than about 2.9×10⁹ TU/kg, less than about 2.8×10⁹ TU/kg, less than about 2.7×10⁹ TU/kg, less than about 2.6×10⁹ TU/kg, less than about 2.5×10⁹ TU/kg, less than about 2.4×10⁹ TU/kg, less than about 2.3×10⁹ TU/kg, less than about 2.2×10⁹ TU/kg, less than about 2.1×10⁹ TU/kg, less than about 2.0×10⁹ TU/kg, less than about 1.9×10⁹ TU/kg, less than about 1.8×10⁹ TU/kg, less than about 1.7×10⁹ TU/kg, less than about 1.6×10⁹ TU/kg, less than about 1.5×10⁹ TU/kg, less than about 1.4×10⁹ TU/kg, less than about 1.3×10⁹ TU/kg, less than about 1.2×10⁹ TU/kg, less than about 1.1×10⁹ TU/kg, or less than about 1.0×10⁹ TU/kg.

In some embodiments, the dose of the lentiviral vector is less than about 9.9×10⁸ TU/kg, less than about 9.8×10⁸ TU/kg, less than about 9.7×10⁸ TU/kg, less than about 9.6×10⁸ TU/kg, less than about 9.5×10⁸ TU/kg, less than about 9.4×10⁸ TU/kg, less than about 9.3×10⁸ TU/kg, less than about 9.2×10⁸ TU/kg, less than about 9.1×10⁸ TU/kg, less than about 9.0×10⁸ TU/kg, less than about 8.9×10⁸ TU/kg, less than about 8.8×10⁸ TU/kg, less than about 8.7×10⁸ TU/kg, less than about 8.6×10⁸ TU/kg, less than about 8.5×10⁸ TU/kg, less than about 8.4×10⁸ TU/kg, less than about 8.3×10⁸ TU/kg, less than about 8.2×10⁸ TU/kg, less than about 8.1×10⁸ TU/kg, less than about 8.0×10⁸ TU/kg, less than about 7.9×10⁸ TU/kg, less than about 7.8×10⁸ TU/kg, less than about 7.7×10⁸ TU/kg, less than about 7.6×10⁸ TU/kg, less than about 7.5×10⁸ TU/kg, less than about 7.4×10⁸ TU/kg, less than about 7.3×10⁸ TU/kg, less than about 7.2×10⁸ TU/kg, less than about 7.1×10⁸ TU/kg, less than about 7.0×10⁸ TU/kg, less than about 6.9×10⁸ TU/kg, less than about 6.8×10⁸ TU/kg, less than about 6.7×10⁸ TU/kg, less than about 6.6×10⁸ TU/kg, less than about 6.5×10⁸ TU/kg, less than about 6.4×10⁸ TU/kg, less than about 6.3×10⁸ TU/kg, less than about 6.2×10⁸ TU/kg, less than about 6.1×10⁸ TU/kg, less than about 6.0×10⁸ TU/kg, less than about 5.9×10⁸ TU/kg, less than about 5.8×10⁸ TU/kg, less than about 5.7×10⁸ TU/kg, less than about 5.6×10⁸ TU/kg, less than about 5.5×10⁸ TU/kg, less than about 5.4×10⁸ TU/kg, less than about 5.3×10⁸ TU/kg, less than about 5.2×10⁸ TU/kg, less than about 5.1×10⁸ TU/kg, less than about 5.0×10⁸ TU/kg, less than about 4.9×10⁸ TU/kg, less than about 4.8×10⁸ TU/kg, less than about 4.7×10⁸ TU/kg, less than about 4.6×10⁸ TU/kg, less than about 4.5×10⁸ TU/kg, less than about 4.4×10⁸ TU/kg, less than about 4.3×10⁸ TU/kg, less than about 4.2×10⁸ TU/kg, less than about 4.1×10⁸ TU/kg, less than about 4.0×10⁸ TU/kg, less than about 3.9×10⁸ TU/kg, less than about 3.8×10⁸ TU/kg, less than about 3.7×10⁸ TU/kg, less than about 3.6×10⁸ TU/kg, less than about 3.5×10⁸ TU/kg, less than about 3.4×10⁸ TU/kg, less than about 3.3×10⁸ TU/kg, less than about 3.2×10⁸ TU/kg, less than about 3.1×10⁸ TU/kg, less than about 3.0×10⁸ TU/kg, less than about 2.9×10⁸ TU/kg, less than about 2.8×10⁸ TU/kg, less than about 2.7×10⁸ TU/kg, less than about 2.6×10⁸ TU/kg, less than about 2.5×10⁸ TU/kg, less than about 2.4×10⁸ TU/kg, less than about 2.3×10⁸ TU/kg, less than about 2.2×10⁸ TU/kg, less than about 2.1×10⁸ TU/kg, less than about 2.0×10⁸ TU/kg, less than about 1.9×10⁸ TU/kg, less than about 1.8×10⁸ TU/kg, less than about 1.7×10⁸ TU/kg, less than about 1.6×10⁸ TU/kg, less than about 1.5×10⁸ TU/kg, less than about 1.4×10⁸ TU/kg, less than about 1.3×10⁸ TU/kg, less than about 1.2×10⁸ TU/kg, less than about 1.1×10⁸ TU/kg, or less than about 1.0×10⁸ TU/kg.

In some embodiments, the dose of the lentiviral vector is between about 1×10⁸ TU/kg and about 5×10¹⁰ TU/kg, between about 1.5×10⁸ TU/kg and about 5×10¹⁰ TU/kg, between about 2×10⁸ TU/kg and about 5×10¹⁰ TU/kg, between about 2.5×10⁸ TU/kg and about 5×10¹⁰ TU/kg, between about 3×10⁸ TU/kg and about 5×10¹⁰ TU/kg, between about 3.5×10⁸ TU/kg and about 5×10¹⁰ TU/kg, between about 4×10⁸ TU/kg and about 5×10¹⁰ TU/kg, between about 4.5×10⁸ TU/kg and about 5×10¹⁰ TU/kg, between about 5×10⁸ TU/kg and about 5×10¹⁰ TU/kg, between about 5.5×10⁸ TU/kg and about 5×10¹⁰ TU/kg, between about 6×10⁸ TU/kg and about 5×10¹⁰ TU/kg, between about 6.5×10⁸ TU/kg and about 5×10¹⁰ TU/kg, between about 7×10⁸ TU/kg and about 5×10¹⁰ TU/kg, between about 7.5×10⁸ TU/kg and about 5×10¹⁰ TU/kg, between about 8×10⁸ TU/kg and about 5×10¹⁰ TU/kg, between about 8.5×10⁸ TU/kg and about 5×10¹⁰ TU/kg, between about 9×10⁸ TU/kg and about 5×10¹⁰ TU/kg, between about 9.5×10⁸ TU/kg and about 5×10¹⁰ TU/kg, between about 1×10⁹ TU/kg and about 5×10¹⁰ TU/kg, between about 1.5×10⁹ TU/kg and about 5×10¹⁰ TU/kg, between about 2×10⁹ TU/kg and about 5×10¹⁰ TU/kg, between about 2.5×10⁹ TU/kg and about 5×10¹⁰ TU/kg, between about 3×10⁹ TU/kg and about 5×10¹⁰ TU/kg, between about 3.5×10⁹ TU/kg and about 5×10¹⁰ TU/kg, between about 4×10⁹ TU/kg and about 5×10¹⁰ TU/kg, between about 4.5×10⁹ TU/kg and about 5×10¹⁰ TU/kg, between about 5×10⁹ TU/kg and about 5×10¹⁰ TU/kg, between about 5.5×10⁹ TU/kg and about 5×10¹⁰ TU/kg, between about 6×10⁹ TU/kg and about 5×10¹⁰ TU/kg, between about 6.5×10⁹ TU/kg and about 5×10¹⁰ TU/kg, between about 7×10⁹ TU/kg and about 5×10¹⁰ TU/kg, between about 7.5×10⁹ TU/kg and about 5×10¹⁰ TU/kg, between about 8×10⁹ TU/kg and about 5×10¹⁰ TU/kg, between about 8.5×10⁹ TU/kg and about 5×10¹⁰ TU/kg, between about 9×10⁹ TU/kg and about 5×10¹⁰ TU/kg, between about 9.5×10⁹ TU/kg and about 5×10¹⁰ TU/kg, between about 1×10⁹ and about 6×10⁹ TU/kg, between about 2×10⁹ and about 6×10⁹ TU/kg, between about 3×10⁹ and about 6×10⁹ TU/kg, between about 4×10⁹ and about 6×10⁹ TU/kg, between about 5×10⁹ and about 6×10⁹ TU/kg, between about 10¹⁰ TU/kg and about 5×10¹⁰ TU/kg, between about 1.5×10¹⁰ TU/kg and about 5×10¹⁰ TU/kg, between about 2×10¹⁰ TU/kg and about 5×10¹⁰ TU/kg, between about 2.5×10¹⁰ TU/kg and about 5×10¹⁰ TU/kg, between about 3×10¹⁰ TU/kg and about 5×10¹⁰ TU/kg, between about 3.5×10¹⁰ TU/kg and about 5×10¹⁰ TU/kg, between about 4×10¹⁰ TU/kg and about 5×10¹⁰ TU/kg, or between about 4.5×10¹⁰ TU/kg and about 5×10¹⁰ TU/kg.

In some embodiments, the dose of the lentiviral vector is between about 1×10⁸ TU/kg and about 5×10¹⁰ TU/kg, between about 1×10⁸ TU/kg and about 4.5×10¹⁰ TU/kg, between about 1×10⁸ TU/kg and about 4×10¹⁰ TU/kg, between about 1×10⁸ TU/kg and about 3.5×10¹⁰ TU/kg, between about 1×10⁸ TU/kg and about 3×10¹⁰ TU/kg, between about 1×10⁸ TU/kg and about 2.5×10¹⁰ TU/kg, between about 1×10⁸ TU/kg and about 2×10¹⁰ TU/kg, between about 1×10⁸ TU/kg and about 1.5×10¹⁰ TU/kg, between about 1×10⁸ TU/kg and about 10¹⁰ TU/kg, between about 1×10⁸ TU/kg and about 9×10⁹ TU/kg, between about 1×10⁸ TU/kg and about 8.5×10⁹ TU/kg, between about 1×10⁸ TU/kg and about 8×10⁹ TU/kg, between about 1×10⁸ TU/kg and about 7.5×10⁹ TU/kg, between about 1×10⁸ TU/kg and about 7×10⁹ TU/kg, between about 1×10⁸ TU/kg and about 6.5×10⁹ TU/kg, between about 1×10⁸ TU/kg and about 6×10⁹ TU/kg, between about 1×10⁸ TU/kg and about 5.5×10⁹ TU/kg, between about 1×10⁸ TU/kg and about 5×10⁹ TU/kg, between about 1×10⁸ TU/kg and about 4.5×10⁹ TU/kg, between about 1×10⁸ TU/kg and about 4×10⁹ TU/kg, between about 1×10⁸ TU/kg and about 3.5×10⁹ TU/kg, between about 1×10⁸ TU/kg and about 3×10⁹ TU/kg, between about 1×10⁸ TU/kg and about 2.5×10⁹ TU/kg, between about 1×10⁸ TU/kg and about 2×10⁹, between about 1×10⁸ TU/kg and about 1.5×10⁹ TU/kg, between about 1×10⁸ TU/kg and about 1×10⁹ TU/kg, between about 1×10⁸ TU/kg and about 9.5×10⁸ TU/kg, between about 1×10⁸ TU/kg and about 9×10⁸ TU/kg, between about 1×10⁸ TU/kg and about 8.5×10⁸ TU/kg, between about 1×10⁸ TU/kg and about 8×10⁸ TU/kg, between about 1×10⁸ TU/kg and about 7.5×10⁸ TU/kg, between about 1×10⁸ TU/kg and about 7×10⁸ TU/kg, between about 1×10⁸ TU/kg and about 6.5×10⁸ TU/kg, between about 1×10⁸ TU/kg and about 6×10⁸ TU/kg, between about 1×10⁸ TU/kg and about 5.5×10⁸ TU/kg, between about 1×10⁸ TU/kg and about 5×10⁸ TU/kg, between about 1×10⁸ TU/kg and about 4.5×10⁸ TU/kg, between about 1×10⁸ TU/kg and about 4×10⁸ TU/kg, between about 1×10⁸ TU/kg and about 3.5×10⁸ TU/kg, between about 1×10⁸ TU/kg and about 3×10⁸ TU/kg, between about 1×10⁸ TU/kg and about 2.5×10⁸ TU/kg, between about 1×10⁸ TU/kg and about 2×10⁸, or between about 1×10⁸ TU/kg and about 1.5×10⁸ TU/kg,

In some embodiments, the dose of the lentiviral vector is between about 1×10¹⁰ TU/kg and about 2×10¹⁰ TU/kg, between about 1.1×10¹⁰ TU/kg and about 1.9×10¹⁰ TU/kg, between about 1.2×10¹⁰ TU/kg and about 1.8×10¹⁰ TU/kg, between about 1.3×10¹⁰ TU/kg and about 1.7×10¹⁰ TU/kg, or between about 1.4×10¹⁰ TU/kg and about 1.6×10¹⁰ TU/kg. In some embodiments, the dose of the lentiviral vector is 1.5×10¹⁰ TU/kg. In some embodiments, the dose of the lentiviral vector is about 2×10¹⁰ TU/kg. In some embodiments, the dose of the lentiviral vector is 2×10¹⁰ TU/kg. In some embodiments, the dose of the lentiviral vector is about 6×10¹⁰ TU/kg. In some embodiments, the dose of the lentiviral vector is 6×10¹⁰ TU/kg.

In some embodiments, the dose of the lentiviral vector is between about 1×10⁹ TU/kg and about 2×10⁹ TU/kg, between about 1.1×10⁹ TU/kg and about 1.9×10⁹ TU/kg, between about 1.2×10⁹ TU/kg and about 1.8×10⁹ TU/kg, between about 1.3×10⁹ TU/kg and about 1.7×10⁹ TU/kg, or between about 1.4×10⁹ TU/kg and about 1.6×10⁹ TU/kg. In certain embodiments, the dose of the lentiviral vector is between about 4×10⁹ and about 6×10⁹ TU/kg. In some embodiments, the dose of the lentiviral vector is 1.5×10⁹ TU/kg. In some embodiments, the dose of the lentiviral vector is 4×10⁹ TU/kg. In some embodiments, the dose of the lentiviral vector is 4.5×10⁹ TU/kg. In some embodiments, the dose of the lentiviral vector is 5×10⁹ TU/kg. In some embodiments, the dose of the lentiviral vector is 5.5×10⁹ TU/kg. In some embodiments, the dose of the lentiviral vector is about 6×10⁹ TU/kg. In some embodiments, the dose of the lentiviral vector is 6×10⁹ TU/kg.

In certain embodiments, the dose of the lentiviral vector is about 2.5×10⁹ TU/kg. In certain embodiments, the dose of the lentiviral vector is 2.5×10⁹ TU/kg. In certain embodiments, the dose of the lentiviral vector is about 3.0×10⁹ TU/kg. In certain embodiments, the dose of the lentiviral vector is 3.0×10⁹ TU/kg. In certain embodiments, the dose of the lentiviral vector is about 7.5×10⁹ TU/kg. In certain embodiments, the dose of the lentiviral vector is 7.5×10⁹ TU/kg. In some embodiments, the dose of the lentiviral vector is about 2×10¹⁰ TU/kg. In some embodiments, the dose of the lentiviral vector is 2×10¹⁰ TU/kg. In some embodiments, the dose of the lentiviral vector is about 6×10¹⁰ TU/kg. In some embodiments, the dose of the lentiviral vector is 6×10¹⁰ TU/kg.

In some embodiments, the lentiviral vector is administered as a single dose or multiple doses. In some embodiments, the lentiviral vector dose is administered at once or divided into multiple sub-dose, e.g., two sub-doses, three sub-doses, four sub-doses, five sub-doses, six sub-doses, or more than six sub-doses. In some embodiments, more than one lentiviral vector is administered.

In some embodiments, the dose of lentiviral vector is administered at least twice, at least three times, at least four times, at least five times, at least six times, at least seven times, at least eight times, at least nine times, or at least ten times. In some embodiments, the dose of the lentiviral vector is administered once about every week, once about every two weeks, once about every three weeks, or once about every four weeks. In some embodiments, the dose of the lentiviral vector is administered once about every 10 days, once about every 14 days, once about every two weeks, once about every 15 days, once about every three weeks, once about every 20 days, once about every four weeks, once about every month, twice about every month, once about every 5 weeks, once about every 6 weeks, once about every 7 weeks, once about every 8 weeks, once about every 2 months, once about every 9 weeks, once about every 10 weeks, once about every 11 weeks, once about every 12 weeks, once about every 3 months, once about every 13 weeks, once about every 14 weeks, once about every 15 weeks, once about every 16 weeks, once about every 4 months, once about every 17 weeks, once about every 18 weeks, once about every 19 weeks, once about every 20 weeks, once about every 5 months, once about every 21 weeks, once about every 22 weeks, once about every 23 weeks, once about every 24 weeks, once about every 25 weeks, once about every 26 weeks, or once about every 6 months.

In some embodiments, a first dose is administered of the lentiviral vector, the subject is monitored for transgene expression, and a second dose is administered to the subject if the subject has transgene expression that is below a predetermined threshold. In certain embodiments, the subject is administered a second (third, fourth, fifth, sixth, seventh, eighth, ninth, or tenth) dose of the lentiviral vector if less than about 100%, less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 4%, less than about 3%, less than about 3%, or less than about 1% of target cells express the transgene. In some embodiments, the subject is administered a second (third, fourth, fifth, sixth, seventh, eighth, ninth, or tenth) dose of the lentiviral vector if less than about 50% of target cells express the transgene. In some embodiments, the subject is administered a second (third, fourth, fifth, sixth, seventh, eighth, ninth, or tenth) dose of the lentiviral vector if less than about 25% of target cells express the transgene. In some embodiments, the subject is administered a second (third, fourth, fifth, sixth, seventh, eighth, ninth, or tenth) dose of the lentiviral vector if less than about 10% of target cells express the transgene. In some embodiments, the subject is administered a second (third, fourth, fifth, sixth, seventh, eighth, ninth, or tenth) dose of the lentiviral vector if less than about 5% of target cells express the transgene. In some embodiments, the subject is administered a second (third, fourth, fifth, sixth, seventh, eighth, ninth, or tenth) dose of the lentiviral vector if less than about 4% of target cells express the transgene. In some embodiments, the subject is administered a second (third, fourth, fifth, sixth, seventh, eighth, ninth, or tenth) dose of the lentiviral vector if less than about 3% of target cells express the transgene. In some embodiments, the subject is administered a second (third, fourth, fifth, sixth, seventh, eighth, ninth, or tenth) dose of the lentiviral vector if less than about 2% of target cells express the transgene. In some embodiments, the subject is administered a second (third, fourth, fifth, sixth, seventh, eighth, ninth, or tenth) dose of the lentiviral vector if less than about 1% of target cells express the transgene.

In some embodiments, the lentiviral vector is administered via intravenous injection. In some embodiments, the lentiviral vector is administered via non-intravenous injection (e.g., subcutaneously or intradermally).

In some embodiments, the subject is a pediatric subject, whereas in other aspects, the subject is an adult subject. In some embodiments, the subject is a male. In other embodiments, the subject is a female.

The lentiviral vectors disclosed herein can be used at low or reduced dosages (e.g., 10¹⁰ TU/kg or lower, 10⁹ TU/kg or lower, or 10⁸ TU/kg or lower) in vivo in a mammal, e.g., a human patient, using a gene therapy approach to treatment of a bleeding disease or disorder selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath would be therapeutically beneficial. In one embodiment, the bleeding disease or disorder is hemophilia. In another embodiment, the bleeding disease or disorder is hemophilia A.

In some embodiments, target cells (e.g., hepatocytes) are treated in vitro with low doses (e.g., 10¹⁰ TU/kg or lower, 10⁹ TU/kg or lower, or 10⁸ TU/kg or lower) of the lentiviral vectors disclosed herein before being administered to the patient. In certain embodiments, target cells (e.g., hepatocytes) are treated in vitro with about 3.0×10⁹ TU/kg of the lentiviral vectors disclosed herein before being administered to the patient. In yet another embodiment, cells from the patient (e.g., hepatocytes) are treated ex vivo with low doses (e.g., 10¹⁰ TU/kg or lower, 10⁹ TU/kg or lower, or 10⁸ TU/kg or lower) of the lentiviral vectors disclosed herein before being administered to the patient.

In some embodiments, plasma FIX activity post administration of a lentiviral vectors disclosed herein (administered, e.g., at 10¹⁰ TU/kg or lower, 10⁹ TU/kg or lower, or 10⁸ TU/kg or lower) is increased by at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, or at least about 300%, relative to physiologically normal circulating FIX levels.

In one embodiment, the plasma FIX activity post administration of a lentiviral vector of the present disclosure is increased by at least about 3,000% to about 5,000% relative to physiologically normal circulating FIX levels. In some embodiments, at 21 days post administration of a lentiviral vector comprising a codon-optimized gene encoding polypeptides with FIX activity described herein, plasma FIX activity is increased by at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, at least about 180-fold, at least about 190-fold, or at least about 200-fold relative to a subject administered a corresponding lentiviral vector comprising a reference nucleic acid molecule comprising SEQ ID NO: 8 or SEQ ID NO: 9.

The present disclosure also provides methods of treating, preventing, or ameliorating a hemostatic disorder (e.g., a bleeding disorder such as hemophilia A) in a subject in need thereof comprising administering to the subject a therapeutically effective amount of a lentiviral vector comprising an isolated nucleic acid molecule comprising a nucleotide sequence encoding a polypeptide with FIX activity, wherein the lentiviral vector is administered as at least one dose of 5×10¹⁰ or less TU/kg, 10⁹ or less TU/kg, or 10⁸ or less TU/kg.

The treatment, amelioration, and prevention by the lentiviral vector of the present disclosure can be a bypass therapy. The subject receiving bypass therapy can have already developed an inhibitor to a clotting factor, e.g., FIX, or is subject to developing a clotting factor inhibitor.

The lentiviral vectors of the present disclosure treat or prevent a hemostatic disorder by promoting the formation of a fibrin clot. The polypeptide having FIX activity encoded by the nucleic acid molecule of the disclosure can activate a member of a coagulation cascade. The clotting factor can be a participant in the extrinsic pathway, the intrinsic pathway or both.

The lentiviral vectors of the present disclosure can be used to treat hemostatic disorders known to be treatable with FIX. The hemostatic disorders that can be treated using methods of the disclosure include, but are not limited to, hemophilia A, hemophilia B, von Willebrand's disease, Factor XI deficiency (PTA deficiency), Factor XII deficiency, as well as deficiencies or structural abnormalities in fibrinogen, prothrombin, Factor V, Factor VII, Factor X, or Factor XIII, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, and bleeding in the illiopsoas sheath.

In some embodiments, the hemostatic disorder is an inherited disorder. In one embodiment, the subject has hemophilia A. In other embodiments, the hemostatic disorder is the result of a deficiency in FIX. In other embodiments, the hemostatic disorder can be the result of a defective FIX clotting factor.

In another embodiment, the hemostatic disorder can be an acquired disorder. The acquired disorder can result from an underlying secondary disease or condition. The unrelated condition can be, as an example, but not as a limitation, cancer, an autoimmune disease, or pregnancy. The acquired disorder can result from old age or from medication to treat an underlying secondary disorder (e.g., cancer chemotherapy).

The disclosure also relates to methods of treating a subject that does not have a hemostatic disorder or a secondary disease or condition resulting in acquisition of a hemostatic disorder. The disclosure thus relates to a method of treating a subject in need of a general hemostatic agent comprising administering a therapeutically effective amount of a lentiviral vector of the present disclosure. For example, in one embodiment, the subject in need of a general hemostatic agent is undergoing, or is about to undergo, surgery. The lentiviral vector of the disclosure can be administered prior to or after surgery as a prophylactic.

The lentiviral vector of the disclosure can be administered during or after surgery to control an acute bleeding episode. The surgery can include, but is not limited to, liver transplantation, liver resection, or stem cell transplantation.

In another embodiment, the lentiviral vector of the disclosure can be used to treat a subject having an acute bleeding episode who does not have a hemostatic disorder. The acute bleeding episode can result from severe trauma, e.g., surgery, an automobile accident, wound, laceration gun shot, or any other traumatic event resulting in uncontrolled bleeding.

The lentiviral vector can be used to prophylactically treat a subject with a hemostatic disorder. The lentiviral vector can also be used to treat an acute bleeding episode in a subject with a hemostatic disorder.

In some embodiments, a lentiviral vector of the disclosure is administered in combination with at least one other agent that promotes hemostasis. Said other agent that promotes hemostasis in a therapeutic with demonstrated clotting activity. As an example, but not as a limitation, the hemostatic agent can include Factor V, Factor VII, Factor VIII, Factor X, Factor XI, Factor XII, Factor XIII, prothrombin, or fibrinogen or activated forms of any of the preceding. The clotting factor or hemostatic agent can also include anti-fibrinolytic drugs, e.g., epsilon-amino-caproic acid, tranexamic acid.

In one embodiment of the disclosure, the composition (e.g., the lentiviral vector) is one in which the FIX is present in activatable form when administered to a subject. Such an activatable molecule can be activated in vivo at the site of clotting after administration to a subject.

The lentiviral vector of the disclosure can be administered intravenously, subcutaneously, intramuscularly, or via any mucosal surface, e.g., orally, sublingually, buccally, sublingually, nasally, rectally, vaginally or via pulmonary route. The lentiviral vector can be implanted within or linked to a biopolymer solid support that allows for the slow release of the vector to the desired site.

In one embodiment, the route of administration of the lentiviral vectors is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the lentiviral vector can be delivered directly to the site of the adverse cellular population thereby increasing the exposure of the diseased tissue to the therapeutic agent.

### C. The Lentiviral Vector

Certain aspects of the present disclosure are directed to lentiviral vectors, lentiviral vector particles, and/or methods of use thereof, wherein the lentiviral vector comprises a nucleotide sequence encoding a polypeptide with factor IX (FIX) activity. In some embodiments, the polypeptide with FIX activity comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 12. In some embodiments, the polypeptide with FIX activity comprises the amino acid sequence set forth in SEQ ID NO: 12. In some embodiments, the polypeptide with FIX activity is a human FIX. In some embodiments, the polypeptide with FIX activity is a variant of human FIX. In certain embodiments, the polypeptide with FIX activity is a R338L variant of human FIX. In certain embodiments, the polypeptide with FIX activity is the Padua variant.

In some embodiments, the polypeptide with FIX activity is a monomer-dimer hybrid molecule comprising FIX. The term “monomer-dimer hybrid” used herein refers to a chimeric protein comprising a first polypeptide chain and a second polypeptide chain, which are associated with each other by a disulfide bond, wherein the first chain comprises FIX and a first Fc region and the second chain comprises, consists essentially of, or consists of a second Fc region without the FIX. The monomer-dimer hybrid construct thus is a hybrid comprising a monomer aspect having only one clotting factor and a dimer aspect having two Fc regions.

C.1. Nucleotide Sequence Encoding a Polypeptide with FIX Activity

In some embodiments, the nucleotide sequence encoding a polypeptide with FIX activity is codon optimized. In certain embodiments, the nucleotide sequence encoding a polypeptide with FIX activity comprises a nucleic acid sequence that has at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1. In some embodiments, the nucleotide sequence has at least about 85% sequence identity to the nucleotide sequence set forth in SEQ ID NO: 1. In some embodiments, the nucleotide sequence has at least about 90% sequence identity to the nucleotide sequence set forth in SEQ ID NO: 1. In some embodiments, the nucleotide sequence has at least about 91% sequence identity to the nucleotide sequence set forth in SEQ ID NO: 1. In some embodiments, the nucleotide sequence has at least about 92% sequence identity to the nucleotide sequence set forth in SEQ ID NO: 1. In some embodiments, the nucleotide sequence has at least about 93% sequence identity to the nucleotide sequence set forth in SEQ ID NO: 1. In some embodiments, the nucleotide sequence has at least about 94% sequence identity to the nucleotide sequence set forth in SEQ ID NO: 1. In some embodiments, the nucleotide sequence has at least about 95% sequence identity to the nucleotide sequence set forth in SEQ ID NO: 1. In some embodiments, the nucleotide sequence has at least about 96% sequence identity to the nucleotide sequence set forth in SEQ ID NO: 1. In some embodiments, the nucleotide sequence has at least about 97% sequence identity to the nucleotide sequence set forth in SEQ ID NO: 1. In some embodiments, the nucleotide sequence has at least about 98% sequence identity to the nucleotide sequence set forth in SEQ ID NO: 1. In some embodiments, the nucleotide sequence has at least about 99% sequence identity to the nucleotide sequence set forth in SEQ ID NO: 1. In some embodiments, the nucleotide sequence is identical to the nucleotide sequence set forth in SEQ ID NO: 1.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to nucleotides 139-1386 of the nucleotide sequence set forth in SEQ ID NO: 2. In some embodiments, the nucleotide sequence has at least about 85% sequence identity to nucleotides 139-1386 of the nucleotide sequence set forth in SEQ ID NO: 2. In some embodiments, the nucleotide sequence has at least about 90% sequence identity to nucleotides 139-1386 of the nucleotide sequence set forth in SEQ ID NO: 2. In some embodiments, the nucleotide sequence has at least about 91% sequence identity to nucleotides 139-1386 of the nucleotide sequence set forth in SEQ ID NO: 2. In some embodiments, the nucleotide sequence has at least about 92% sequence identity to nucleotides 139-1386 of the nucleotide sequence set forth in SEQ ID NO: 2. In some embodiments, the nucleotide sequence has at least about 93% sequence identity to nucleotides 139-1386 of the nucleotide sequence set forth in SEQ ID NO: 2. In some embodiments, the nucleotide sequence has at least about 94% sequence identity to nucleotides 139-1386 of the nucleotide sequence set forth in SEQ ID NO: 2. In some embodiments, the nucleotide sequence has at least about 95% sequence identity to nucleotides 139-1386 of the nucleotide sequence set forth in SEQ ID NO: 2. In some embodiments, the nucleotide sequence has at least about 96% sequence identity to nucleotides 139-1386 of the nucleotide sequence set forth in SEQ ID NO: 2. In some embodiments, the nucleotide sequence has at least about 97% sequence identity to nucleotides 139-1386 of the nucleotide sequence set forth in SEQ ID NO: 2. In some embodiments, the nucleotide sequence has at least about 98% sequence identity to nucleotides 139-1386 of the nucleotide sequence set forth in SEQ ID NO: 2. In some embodiments, the nucleotide sequence has at least about 99% sequence identity to nucleotides 139-1386 of the nucleotide sequence set forth in SEQ ID NO: 2. In some embodiments, the nucleotide sequence is identical to nucleotides 139-1386 of the nucleotide sequence set forth in SEQ ID NO: 2.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to nucleotides 139-1386 of the nucleotide sequence set forth in SEQ ID NO: 3. In some embodiments, the nucleotide sequence has at least about 85% sequence identity to nucleotides 139-1386 of the nucleotide sequence set forth in SEQ ID NO: 3. In some embodiments, the nucleotide sequence has at least about 90% sequence identity to nucleotides 139-1386 of the nucleotide sequence set forth in SEQ ID NO: 3. In some embodiments, the nucleotide sequence has at least about 91% sequence identity to nucleotides 139-1386 of the nucleotide sequence set forth in SEQ ID NO: 3. In some embodiments, the nucleotide sequence has at least about 92% sequence identity to nucleotides 139-1386 of the nucleotide sequence set forth in SEQ ID NO: 3. In some embodiments, the nucleotide sequence has at least about 93% sequence identity to nucleotides 139-1386 of the nucleotide sequence set forth in SEQ ID NO: 3. In some embodiments, the nucleotide sequence has at least about 94% sequence identity to nucleotides 139-1386 of the nucleotide sequence set forth in SEQ ID NO: 3. In some embodiments, the nucleotide sequence has at least about 95% sequence identity to nucleotides 139-1386 of the nucleotide sequence set forth in SEQ ID NO: 3. In some embodiments, the nucleotide sequence has at least about 96% sequence identity to nucleotides 139-1386 of the nucleotide sequence set forth in SEQ ID NO: 3. In some embodiments, the nucleotide sequence has at least about 97% sequence identity to nucleotides 139-1386 of the nucleotide sequence set forth in SEQ ID NO: 3. In some embodiments, the nucleotide sequence has at least about 98% sequence identity to nucleotides 139-1386 of the nucleotide sequence set forth in SEQ ID NO: 3. In some embodiments, the nucleotide sequence has at least about 99% sequence identity to nucleotides 139-1386 of the nucleotide sequence set forth in SEQ ID NO: 3. In some embodiments, the nucleotide sequence is identical to nucleotides 139-1386 of the nucleotide sequence set forth in SEQ ID NO: 3.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to nucleotides 139-1386 of the nucleotide sequence set forth in SEQ ID NO: 4. In some embodiments, the nucleotide sequence has at least about 85% sequence identity to nucleotides 139-1386 of the nucleotide sequence set forth in SEQ ID NO: 4. In some embodiments, the nucleotide sequence has at least about 90% sequence identity to nucleotides 139-1386 of the nucleotide sequence set forth in SEQ ID NO: 4. In some embodiments, the nucleotide sequence has at least about 91% sequence identity to nucleotides 139-1386 of the nucleotide sequence set forth in SEQ ID NO: 4. In some embodiments, the nucleotide sequence has at least about 92% sequence identity to nucleotides 139-1386 of the nucleotide sequence set forth in SEQ ID NO: 4. In some embodiments, the nucleotide sequence has at least about 93% sequence identity to nucleotides 139-1386 of the nucleotide sequence set forth in SEQ ID NO: 4. In some embodiments, the nucleotide sequence has at least about 94% sequence identity to nucleotides 139-1386 of the nucleotide sequence set forth in SEQ ID NO: 4. In some embodiments, the nucleotide sequence has at least about 95% sequence identity to nucleotides 139-1386 of the nucleotide sequence set forth in SEQ ID NO: 4. In some embodiments, the nucleotide sequence has at least about 96% sequence identity to nucleotides 139-1386 of the nucleotide sequence set forth in SEQ ID NO: 4. In some embodiments, the nucleotide sequence has at least about 97% sequence identity to nucleotides 139-1386 of the nucleotide sequence set forth in SEQ ID NO: 4. In some embodiments, the nucleotide sequence has at least about 98% sequence identity to nucleotides 139-1386 of the nucleotide sequence set forth in SEQ ID NO: 4. In some embodiments, the nucleotide sequence has at least about 99% sequence identity to nucleotides 139-1386 of the nucleotide sequence set forth in SEQ ID NO: 4. In some embodiments, the nucleotide sequence is identical to nucleotides 139-1386 of the nucleotide sequence set forth in SEQ ID NO: 4.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to nucleotides 139-1386 of the nucleotide sequence set forth in SEQ ID NO: 5. In some embodiments, the nucleotide sequence has at least about 85% sequence identity to nucleotides 139-1386 of the nucleotide sequence set forth in SEQ ID NO: 5. In some embodiments, the nucleotide sequence has at least about 90% sequence identity to nucleotides 139-1386 of the nucleotide sequence set forth in SEQ ID NO: 5. In some embodiments, the nucleotide sequence has at least about 91% sequence identity to nucleotides 139-1386 of the nucleotide sequence set forth in SEQ ID NO: 5. In some embodiments, the nucleotide sequence has at least about 92% sequence identity to nucleotides 139-1386 of the nucleotide sequence set forth in SEQ ID NO: 5. In some embodiments, the nucleotide sequence has at least about 93% sequence identity to nucleotides 139-1386 of the nucleotide sequence set forth in SEQ ID NO: 5. In some embodiments, the nucleotide sequence has at least about 94% sequence identity to nucleotides 139-1386 of the nucleotide sequence set forth in SEQ ID NO: 5. In some embodiments, the nucleotide sequence has at least about 95% sequence identity to nucleotides 139-1386 of the nucleotide sequence set forth in SEQ ID NO: 5. In some embodiments, the nucleotide sequence has at least about 96% sequence identity to nucleotides 139-1386 of the nucleotide sequence set forth in SEQ ID NO: 5. In some embodiments, the nucleotide sequence has at least about 97% sequence identity to nucleotides 139-1386 of the nucleotide sequence set forth in SEQ ID NO: 5. In some embodiments, the nucleotide sequence has at least about 98% sequence identity to nucleotides 139-1386 of the nucleotide sequence set forth in SEQ ID NO: 5. In some embodiments, the nucleotide sequence has at least about 99% sequence identity to nucleotides 139-1386 of the nucleotide sequence set forth in SEQ ID NO: 5. In some embodiments, the nucleotide sequence is identical to nucleotides 139-1386 of the nucleotide sequence set forth in SEQ ID NO: 5.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to nucleotides 139-1386 of the nucleotide sequence set forth in SEQ ID NO: 6. In some embodiments, the nucleotide sequence has at least about 85% sequence identity to nucleotides 139-1386 of the nucleotide sequence set forth in SEQ ID NO: 6. In some embodiments, the nucleotide sequence has at least about 90% sequence identity to nucleotides 139-1386 of the nucleotide sequence set forth in SEQ ID NO: 6. In some embodiments, the nucleotide sequence has at least about 91% sequence identity to nucleotides 139-1386 of the nucleotide sequence set forth in SEQ ID NO: 6. In some embodiments, the nucleotide sequence has at least about 92% sequence identity to nucleotides 139-1386 of the nucleotide sequence set forth in SEQ ID NO: 6. In some embodiments, the nucleotide sequence has at least about 93% sequence identity to nucleotides 139-1386 of the nucleotide sequence set forth in SEQ ID NO: 6. In some embodiments, the nucleotide sequence has at least about 94% sequence identity to nucleotides 139-1386 of the nucleotide sequence set forth in SEQ ID NO: 6. In some embodiments, the nucleotide sequence has at least about 95% sequence identity to nucleotides 139-1386 of the nucleotide sequence set forth in SEQ ID NO: 6. In some embodiments, the nucleotide sequence has at least about 96% sequence identity to nucleotides 139-1386 of the nucleotide sequence set forth in SEQ ID NO: 6. In some embodiments, the nucleotide sequence has at least about 97% sequence identity to nucleotides 139-1386 of the nucleotide sequence set forth in SEQ ID NO: 6. In some embodiments, the nucleotide sequence has at least about 98% sequence identity to nucleotides 139-1386 of the nucleotide sequence set forth in SEQ ID NO: 6. In some embodiments, the nucleotide sequence has at least about 99% sequence identity to nucleotides 139-1386 of the nucleotide sequence set forth in SEQ ID NO: 6. In some embodiments, the nucleotide sequence is identical to nucleotides 139-1386 of the nucleotide sequence set forth in SEQ ID NO: 6.

In some embodiments, the nucleotide sequence has at least about 70%, at least about 75%, at least about 80%, at least about 85%, at least about 90%, at least about 95%, at least about 96%, at least about 97%, at least about 98%, at least about 99%, or about 100% sequence identity to nucleotides 139-1386 of the nucleotide sequence set forth in SEQ ID NO: 7. In some embodiments, the nucleotide sequence has at least about 85% sequence identity to nucleotides 139-1386 of the nucleotide sequence set forth in SEQ ID NO: 7. In some embodiments, the nucleotide sequence has at least about 90% sequence identity to nucleotides 139-1386 of the nucleotide sequence set forth in SEQ ID NO: 7. In some embodiments, the nucleotide sequence has at least about 91% sequence identity to nucleotides 139-1386 of the nucleotide sequence set forth in SEQ ID NO: 7. In some embodiments, the nucleotide sequence has at least about 92% sequence identity to nucleotides 139-1386 of the nucleotide sequence set forth in SEQ ID NO: 7. In some embodiments, the nucleotide sequence has at least about 93% sequence identity to nucleotides 139-1386 of the nucleotide sequence set forth in SEQ ID NO: 7. In some embodiments, the nucleotide sequence has at least about 94% sequence identity to nucleotides 139-1386 of the nucleotide sequence set forth in SEQ ID NO: 7. In some embodiments, the nucleotide sequence has at least about 95% sequence identity to nucleotides 139-1386 of the nucleotide sequence set forth in SEQ ID NO: 7. In some embodiments, the nucleotide sequence has at least about 96% sequence identity to nucleotides 139-1386 of the nucleotide sequence set forth in SEQ ID NO: 7. In some embodiments, the nucleotide sequence has at least about 97% sequence identity to nucleotides 139-1386 of the nucleotide sequence set forth in SEQ ID NO: 7. In some embodiments, the nucleotide sequence has at least about 98% sequence identity to nucleotides 139-1386 of the nucleotide sequence set forth in SEQ ID NO: 7. In some embodiments, the nucleotide sequence has at least about 99% sequence identity to nucleotides 139-1386 of the nucleotide sequence set forth in SEQ ID NO: 7. In some embodiments, the nucleotide sequence is identical to nucleotides 139-1386 of the nucleotide sequence set forth in SEQ ID NO: 7.

In certain embodiments, the nucleotide sequence encoding a polypeptide with FIX activity further comprises a nucleic acid sequence encoding a signal peptide. In some embodiments, the nucleic acid sequence encoding a signal peptide has at least 60%, at least 70%, at least 80%, at least 90%, at least 95%, at least 96%, at least 97%, at least 98%, at least 99%, or 100% sequence identity to: (i) nucleotides 1-84 of SEQ ID NO: 2; (ii) nucleotides 1-84 of SEQ ID NO: 3; (iii) nucleotides 1-84 of SEQ ID NO: 4; (iv) nucleotides 1-84 of SEQ ID NO: 5; (v) nucleotides 1-84 of SEQ ID NO: 6; or (vi) nucleotides 1-84 of SEQ ID NO: 7. In some embodiments, the nucleic acid sequence encoding a signal peptide comprises the nucleotide sequence set forth in (i) nucleotides 1-84 of SEQ ID NO: 2; (ii) nucleotides 1-84 of SEQ ID NO: 3; (iii) nucleotides 1-84 of SEQ ID NO: 4; (iv) nucleotides 1-84 of SEQ ID NO: 5; (v) nucleotides 1-84 of SEQ ID NO: 6; or (vi) nucleotides 1-84 of SEQ ID NO: 7.

In certain embodiments, the nucleotide sequence encoding a polypeptide with FIX activity further comprises a nucleic acid sequence encoding a propeptide. In some embodiments, the nucleic acid sequence encoding a propeptide has at least 60%, at least 70%, at least 80%, at least 90%, at least 95%, at least 96%, at least 97%, at least 98%, at least 99%, or 100% sequence identity to: (i) nucleotides 85-138 of SEQ ID NO: 2; (ii) nucleotides 85-138 of SEQ ID NO: 3; (iii) nucleotides 85-138 of SEQ ID NO: 4; (iv) nucleotides 85-138 of SEQ ID NO: 5; (v) nucleotides 85-138 of SEQ ID NO: 6; or (vi) nucleotides 85-138 of SEQ ID NO: 7. In some embodiments, the nucleic acid sequence encoding a propeptide comprises the nucleotide sequence set forth in (i) nucleotides 85-138 of SEQ ID NO: 2; (ii) nucleotides 85-138 of SEQ ID NO: 3; (iii) nucleotides 85-138 of SEQ ID NO: 4; (iv) nucleotides 85-138 of SEQ ID NO: 5; (v) nucleotides 85-138 of SEQ ID NO: 6; or (vi) nucleotides 85-138 of SEQ ID NO: 7.

C.1.a. Heterologous Moieties

In some embodiments, the nucleotide molecule encoding a polypeptide with FIX activity further comprises a nucleic acid sequence encoding at least one heterologous moiety. In some embodiments, the heterologous moiety is fused to the C-terminus or the N-terminus of the polypeptide with FIX activity, wherein the polypeptide has procoagulant activity. In some embodiments, the heterologous moiety is inserted into one or more sites within the polypeptide with FIX activity, wherein the polypeptide has procoagulant activity. In some embodiments, the heterologous moiety is a heterologous polypeptide. In certain aspects, the heterologous moiety is an XTEN. In some aspects, the heterologous moiety comprises at least one XTEN inserted into one or more sites within the polypeptide with FIX activity. In other aspects, the heterologous moiety is a half-life extending moiety (e.g., an in vivo half-life extending moiety), which is inserted within the polypeptide with FIX activity. In some embodiments, the heterologous moiety is inserted within the polypeptide with FIX activity at an insertion site disclosed in International Application Publication No. WO 2017/024060, which is incorporated by reference herein in its entirety. In certain embodiments, the heterologous moiety is inserted within the polypeptide with FIX activity immediately downstream of an amino acid corresponding to of amino acid 103 of SEQ ID NO: 2, amino acid 105 of SEQ ID NO: 2, amino acid 142 of SEQ ID NO: 2, amino acid 149 of SEQ ID NO: 2, amino acid 162 of SEQ ID NO: 2, amino acid 166 of SEQ ID NO: 2, amino acid 174 of SEQ ID NO: 2, amino acid 224 of SEQ ID NO: 2, amino acid 226 of SEQ ID NO: 2, amino acid 228 of SEQ ID NO: 2, amino acid 413 of SEQ ID NO: 2, or any combination thereof.

In some embodiments, the heterologous moiety is an FcRn binding partner (e.g., an Fc, and albumin, or a fragment thereof). In some embodiments, the heterologous moiety is an FcRn binding partner, which is fused to the C-terminus or the N-terminus of the polypeptide with FIX activity.

Non-limiting examples of heterologous moieties (e.g., a half-life extending moiety) include albumin, albumin fragments, Fc fragments of immunoglobulins, FcRn binding partners, the C-terminal peptide (CTP) of the β subunit of human chorionic gonadotropin, a HAP sequence, a transferrin, the PAS polypeptides of U.S. Pat Application No. 20100292130, polyglycine linkers, polyserine linkers, peptides and short polypeptides of 6-40 amino acids of two types of amino acids selected from glycine (G), alanine (A), serine (S), threonine (T), glutamate (E) and proline (P) with varying degrees of secondary structure from less than 50% to greater than 50%, amongst others.

In certain aspects a heterologous moiety increases the in vivo or in vitro half-life of the polypeptide with FIX activity produced from the lentiviral vector of the present disclosure. In other aspects a heterologous moiety facilitates visualization or localization of the polypeptide with FIX activity produced from the lentiviral vector of the present disclosure. Visualization and/or location of the polypeptide with FIX activity can be in vivo, in vitro, ex vivo, or combinations thereof. In other aspects a heterologous moiety increases stability of the polypeptide with FIX activity produced from the lentiviral vector of the present disclosure. As used herein, the term “stability” refers to an art-recognized measure of the maintenance of one or more physical properties of the polypeptide with FIX activity in response to an environmental condition (e.g., an elevated or lowered temperature). In certain aspects, the physical property is the maintenance of the covalent structure of the polypeptide with FIX activity (e.g., the absence of proteolytic cleavage, unwanted oxidation or deamidation). In other aspects, the physical property can also be the presence of the polypeptide with FIX activity in a properly folded state (e.g., the absence of soluble or insoluble aggregates or precipitates).

In certain aspects, a heterologous moiety which increases half-life of the FIX fusion protein of the disclosure comprises, without limitation, a heterologous polypeptide such as an albumin, an immunoglobulin Fc region, an XTEN sequence, the C-terminal peptide (CTP) of the β subunit of human chorionic gonadotropin, a PAS sequence, a HAP sequence, a CTP peptide sequence, a transferrin, albumin-binding moiety, or any fragments, derivatives, variants, or combinations of these polypeptides. In other related aspects a heterologous moiety can include an attachment site for a non-polypeptide moiety such as polyethylene glycol (PEG), hydroxyethyl starch (HES), polysialic acid, or any derivatives, variants, or combinations of these moieties.

In certain aspects, a polypeptide with FIX activity of the disclosure comprises one, two, three or more heterologous moieties, which can each be the same or different molecules. In some embodiments, the lentiviral vector comprises one or more nucleotide sequences encoding XTENs. In other embodiments, the lentiviral vector comprises one or more nucleotide sequences encoding XTENs and one or more Fc domains. In one particular embodiment, the lentiviral vector comprises a nucleotide sequence encoding an XTEN inserted within the nucleotide sequence encoding the polypeptide with FIX activity and nucleotide sequence encoding an Fc fused to portion of the nucleotide sequence encoding the C-terminus of the polypeptide with FIX activity.

C.1.a.i. XTENs

In some embodiments, the at least one heterologous moiety is an XTEN. As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a fusion protein partner, XTENs can serve as a carrier, conferring certain desirable pharmacokinetic, physicochemical and pharmaceutical properties when linked to a FIX sequence of the disclosure to create a fusion protein. Such desirable properties include but are not limited to enhanced pharmacokinetic parameters and solubility characteristics. As used herein, “XTEN” specifically excludes antibodies or antibody fragments such as single-chain antibodies or Fc fragments of a light chain or a heavy chain.

In certain aspects, a lentiviral vector of the disclosure comprises at least one nucleotide sequence encoding an XTEN or fragment, variant, or derivative thereof, wherein the nucleotide sequence encoding the XTEN is inserted into the nucleotide sequence encoding the polypeptide with FIX activity, wherein the resulting fusion polypeptide has procoagulant activity. In certain aspects, two of the heterologous moieties are XTEN sequences. In some aspects, three of the heterologous moieties are XTEN sequences. In some aspects, four of the heterologous moieties are XTEN sequences. In some aspects, five of the heterologous moieties are XTEN sequences. In some aspects, six or more of the heterologous moieties are XTEN sequences.

In some embodiments, the XTEN sequence is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues. In one particular embodiment, the XTEN comprises an amino acid sequence of longer than 42 amino acids and shorter than 144 amino acids in length.

The XTEN sequence can comprise one or more sequence motif of 5 to 14 (e.g., 9 to 14) amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids (e.g., 5 amino acids) selected from the group consisting of glycine (G), alanine (A), serine (S), threonine (T), glutamate (E) and proline (P). See US 2010-0239554 A1.

Examples of XTEN sequences that can be used according to the present disclosure are disclosed in US Patent Publication No. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, WO 2011028344 A2, WO 2014/011819 A2, WO 2015/023891, or WO 2017/024060, each of which is incorporated by reference herein in its entirety.

C.1.a.ii. Fc Regions or FcRn Binding Partners

In some embodiments, the at least one heterologous moiety is an Fc region (e.g., an FcRn binding partner) or a fragment thereof. In certain aspects, a lentiviral vector of the disclosure comprises at least one nucleotide sequence encoding an Fc region (e.g., an FcRn binding partner), which is inserted within the nucleotide sequence encoding the polypeptide with FIX activity, fused to the portion of the nucleotide sequence encoding the C-terminus of the polypeptide with FIX activity, or both, wherein the resulting fusion polypeptide has procoagulant activity. “Fc” or “Fc region” as used herein, can be a functional neonatal Fc receptor (FcRn) binding partner comprising an Fc domain, variant, or fragment thereof, unless otherwise specified. An FcRn binding partner is any molecule that can be specifically bound by the FcRn receptor with consequent active transport by the FcRn receptor of the FcRn binding partner, including, but not limited to, albumin. Thus, the term Fc includes any variants of IgG Fc that are functional. The region of the Fc portion of IgG that binds to the FcRn receptor has been described based on X-ray crystallography (Burmeister et al., Nature 372:379 (1994), incorporated herein by reference in its entirety). The major contact area of the Fc with the FcRn is near the junction of the CH2 and CH3 domains. Fc-FcRn contacts are all within a single Ig heavy chain. FcRn binding partners include, but are not limited to, whole IgG, the Fc fragment of IgG, and other fragments of IgG that include the complete binding region of FcRn. An Fc can comprise the CH2 and CH3 domains of an immunoglobulin with or without the hinge region of the immunoglobulin. Also included are Fc fragments, variants, or derivatives which maintain the desirable properties of an Fc region in a fusion protein, e.g., an increase in half-life, e.g., in vivo half-life. Myriad mutants, fragments, variants, and derivatives are described, e.g., in PCT Publication Nos. WO 2011/069164 A2, WO 2012/006623A2, WO 2012/006635A2, or WO 2012/006633A2, all of which are incorporated herein by reference in their entireties.

The nucleotide sequence encoding the one or more Fc domains can be inserted within the nucleotide sequence encoding the polypeptide with FIX activity, fused to portion of the nucleotide sequence encoding the C-terminus of the polypeptide with FIX activity, or both. In some embodiments, the nucleotide sequence encoding the Fc domain is fused to 5′ end of the nucleotide sequence encoding the polypeptide with FIX activity. In some embodiments, the nucleotide sequence encoding the Fc domain is fused to 3′ end of the nucleotide sequence encoding the polypeptide with FIX activity. In some embodiments, the nucleotide sequence encoding the Fc domain is fused to a nucleotide sequence encoding another heterologous moiety, such as an XTEN, which is inserted within the nucleotide sequence encoding the polypeptide with FIX activity or fused to portion of the nucleotide sequence encoding the C-terminus of nucleotide sequence encoding the XTEN. In some embodiments, the lentiviral vector comprises a nucleotide sequence encoding a second Fc domain. The expressed second Fc domain can be associated with the first Fc domain, e.g., through one or more covalent bonds.

C.1.a.iii. Albumins

In some embodiments, the at least one heterologous moiety is an albumin, an albumin binding domain, or an albumin binding small molecule, or a variant, derivative, or fragment thereof. In certain aspects, a lentiviral vector of the disclosure comprises at least one nucleotide sequence encoding an albumin polypeptide or fragment, variant, or derivative thereof, which is inserted within the nucleotide sequence encoding the polypeptide with FIX activity, fused to the portion of the nucleotide sequence encoding the C-terminus of the polypeptide with FIX activity, or both, wherein the resulting fusion polypeptide has procoagulant activity. Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, which are incorporated herein by reference in their entireties.

The albumin-binding polypeptides (ABPs) can compromise, without limitation, bacterial albumin-binding domains, albumin-binding peptides, or albumin-binding antibody fragments that can bind to albumin. Domain 3 from streptococcal protein G, as disclosed by Kraulis et al., FEBS Lett. 378:190-194 (1996) and Linhult et al., Protein Sci. 11:206-213 (2002) is an example of a bacterial albumin-binding domain. Examples of albumin-binding peptides include a series of peptides having the core sequence DICLPRWGCLW (SEQ ID NO: 15). See, e.g., Dennis et al., J. Biol. Chem. 2002, 277: 35035-35043 (2002). Examples of albumin-binding antibody fragments are disclosed in Muller and Kontermann, Curr. Opin. Mol. Ther. 9:319-326 (2007); Roovers et al., Cancer Immunol. Immunother. 56:303-317 (2007), and Holt et al., Prot. Eng. Design Sci., 21:283-288 (2008), which are incorporated herein by reference in their entireties.

In certain aspects, a lentiviral vector of the disclosure comprises at least one nucleotide sequence encoding for an attachment site for a non-polypeptide small molecule, variant, or derivative thereof that can bind to albumin (e.g., an albumin binding small molecule), which is inserted within the nucleotide sequence encoding the polypeptide with FIX activity, fused to the portion of the nucleotide sequence encoding the C-terminus of the polypeptide with FIX activity, or both, wherein the resulting fusion polypeptide has procoagulant activity. An example of such albumin-binding moieties is 2-(3-maleimidopropanamido)-6-(4-(4-iodophenyl)butanamido)hexanoate (“Albu” tag) as disclosed by Trussel et al., Bioconjugate Chem. 20:2286-2292 (2009).

In some embodiments, the albumin-binding polypeptide sequence in the expressed polypeptide is flanked at the C-terminus, the N-terminus, or both termini, by a Gly-Ser peptide linker sequence. In some embodiments, the Gly-Ser peptide linker is Gly₄Ser (SEQ ID NO: 16). In other embodiments, the Gly-Ser peptide linker is (Gly₄Ser)₂ (SEQ ID NO: 17).

C.1.a.iv. CTP

In some embodiments, the at least one heterologous moiety is a C-terminal peptide (CTP) of the β subunit of human chorionic gonadotropin or fragment, variant, or derivative thereof. In certain aspects, a lentiviral vector of the disclosure comprises at least one nucleotide sequence encoding a CTP or fragment, variant, or derivative thereof, which is inserted within the nucleotide sequence encoding the polypeptide with FIX activity, fused to the portion of the nucleotide sequence encoding the C-terminus of the polypeptide with FIX activity, or both, wherein the resulting fusion polypeptide has procoagulant activity. Insertion of one or more CTP peptides into a recombinant protein is known to increase the half-life of that protein. See, e.g., U.S. Pat. No. 5,712,122, incorporated by reference herein in its entirety. Exemplary CTP peptides include DPRFQDSSSSKAPPPSLPSPSRLPGPSDTPIL (SEQ ID NO: 18) or SSSSKAPPPSLPSPSRLPGPSDTPILPQ (SEQ ID NO: 19). See, e.g., U.S. Patent Application Publication No. US 2009/0087411 A1, incorporated by reference. In some embodiments, the CTP sequence in the expressed polypeptide is flanked at the C-terminus, the N-terminus, or both termini, by a Gly-Ser peptide linker sequence. In some embodiments, the Gly-Ser peptide linker is Gly₄Ser (SEQ ID NO: 16). In other embodiments, the Gly-Ser peptide linker is (Gly₄Ser)₂ (SEQ ID NO: 17).

C.1.a.v. PAS

In some embodiments, the at least one heterologous moiety is a PAS peptide. In certain aspects, a lentiviral vector of the disclosure comprises at least one nucleotide sequence encoding a PAS peptide or fragment, variant, or derivative thereof, which is inserted within the nucleotide sequence encoding the polypeptide with FIX activity, fused to the portion of the nucleotide sequence encoding the C-terminus of the polypeptide with FIX activity, or both, wherein the resulting fusion polypeptide has procoagulant activity. A “PAS peptide” or “PAS sequence,” as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the fusion protein. An amino acid polymer also can form random coil conformation when residues other than alanine, serine, and proline are added as a minor constituent in the PAS sequence. By “minor constituent” is meant that that amino acids other than alanine, serine, and proline can be added in the PAS sequence to a certain degree, e.g., up to about 12%, i.e., about 12 of 100 amino acids of the PAS sequence, up to about 10%, up to about 9%, up to about 8%, about 6%, about 5%, about 4%, about 3%, i.e. about 2%, or about 1%, of the amino acids. The amino acids different from alanine, serine and proline can be selected from the group consisting of Arg, Asn, Asp, Cys, Gln, Glu, Gly, His, lie, Leu, Lys, Met, Phe, Thr, Trp, Tyr, and Val. Under physiological conditions, a PAS peptide forms a random coil conformation and thereby can mediate an increased in vivo and/or in vitro stability.

Non-limiting examples of the PAS peptides include ASPAAPAPASPAAPAPSAPA (SEQ ID NO: 20), AAPASPAPAAPSAPAPAAPS (SEQ ID NO: 21), APSSPSPSAPSSPSPASPSS (SEQ ID NO: 22), APSSPSPSAPSSPSPASPS (SEQ ID NO: 23), SSPSAPSPSSPASPSPSSPA (SEQ ID NO: 24), AASPAAPSAPPAAASPAAPSAPPA (SEQ ID NO: 25), ASAAAPAAASAAASAPSAAA (SEQ ID NO: 26) or any variants, derivatives, fragments, or combinations thereof. Additional examples of PAS sequences are known from, e.g., US Pat. Publ. No. 2010/0292130 A1 and PCT Appl. Publ. No. WO 2008/155134 A1. European issued patent EP2173890.

In some embodiments, the PAS sequence in the expressed polypeptide is flanked at the C-terminus, the N-terminus, or both termini, by a Gly-Ser peptide linker sequence. In some embodiments, the Gly-Ser peptide linker is Gly₄Ser (SEQ ID NO: 16). In other embodiments, the Gly/Ser peptide linker is (Gly₄Ser)₂ (SEQ ID NO: 17).

C.1.a.vi. HAP

In some embodiments, the at least one heterologous moiety is a homo-amino acid polymer (HAP) peptide or fragment, variant, or derivative thereof. In certain aspects, a lentiviral vector of the disclosure comprises at least one nucleotide sequence encoding a homo-amino acid polymer (HAP) peptide or fragment, variant, or derivative thereof, which is inserted within the nucleotide sequence encoding the polypeptide with FIX activity, fused to the portion of the nucleotide sequence encoding the C-terminus of the polypeptide with FIX activity, or both, wherein the resulting fusion polypeptide has procoagulant activity. A HAP peptide can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. A HAP sequence is capable of extending half-life of a moiety fused to or linked to the HAP sequence. Non-limiting examples of the HAP sequence include, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200. See, e.g., Schlapschy M et al., Protein Eng. Design Selection, 20: 273-284 (2007).

### C.2. Regulatory Elements

In some embodiments, the lentiviral vector comprises a gene expression control element. The gene expression control sequence can, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus.

Other constitutive promoters can also be used. The promoters useful as gene expression sequences of the disclosure also include inducible promoters. Inducible promoters are expressed in the presence of an inducing agent. For example, the metallothionein promoter is induced to promote transcription and translation in the presence of certain metal ions. Other inducible can be used.

In one embodiment, the disclosure includes expression of a transgene under the control of a tissue specific promoter and/or enhancer. In another embodiment, the promoter or other expression control sequence selectively enhances expression of the transgene in liver cells. Examples of liver specific promoters include, but are not limited to, a mouse thyretin promoter (mTTR), an endogenous human factor VIII promoter (F8), human alpha-1-antitrypsin promoter (hAAT), human albumin minimal promoter, and mouse albumin promoter. In a particular embodiment, the promoter comprises a mTTR promoter. The mTTR promoter is described in R. H. Costa et al., 1986, Mol. Cell. Biol. 6:4697. The F8 promoter is described in Figueiredo and Brownlee, 1995, J. Biol. Chem. 270:11828-11838. In some embodiments, the lentiviral vector comprises at least one tissue specific promoter, i.e., a promoter that would regulate the expression of the polypeptide with FIX activity in a particular tissue or cell type. In some embodiments, a tissue specific promoter in the lentiviral vector selectively enhances expression of the polypeptide with FIX activity in a target liver cell. In some embodiments, the tissue specific promoter that selectively enhances expression of the polypeptide with FIX activity in a target liver cell comprises an APOA2 promoter, SERPINA1 (hAAT) promoter, mTTR promoter, MIR122 promoter, the ET promoter (GenBank No. AY661265; see also Vigna et al., Molecular Therapy 11(5):763 (2005)), or any combination thereof. In some embodiments, the target liver cell is a hepatocyte.

Expression levels can be further enhanced to achieve therapeutic efficacy using one or more enhancers. One or more enhancers can be provided either alone or together with one or more promoter elements. Typically, the expression control sequence comprises a plurality of enhancer elements and a tissue specific promoter. In one embodiment, an enhancer comprises one or more copies of the α-1-microglobulin/bikunin enhancer (Rouet et al., 1992, J. Biol. Chem. 267:20765-20773; Rouet et al., 1995, Nucleic Acids Res. 23:395-404; Rouet et al., 1998, Biochem. J. 334:577-584; III et al., 1997, Blood Coagulation Fibrinolysis 8:S23-S30). In another embodiment, an enhancer is derived from liver specific transcription factor binding sites, such as EBP, DBP, HNF1, HNF3, HNF4, HNF6, with Enh1, comprising HNF1, (sense)-HNF3, (sense)-HNF4, (antisense)-HNF1, (antisense)-HNF6, (sense)-EBP, (antisense)-HNF4 (antisense).

Examples of other suitable vectors and gene regulatory elements are described in WO 02/092134, EP1395293, or U.S. Pat. Nos. 6,808,905, 7,745,179, or 7,179,903, which are incorporated by reference herein in their entireties.

In general, the expression control sequences shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined coding nucleic acid. The gene expression sequences optionally include enhancer sequences or upstream activator sequences as desired.

Since the lentiviral vector can transduce all liver cell types, the expression of the transgene (e.g., FIX) in different cell types can be controlled by using different promoters in the lentiviral vector. Thus, the lentiviral vector can comprise specific promoters which would control expression of the FIX transgene in different tissues or cells types, such as different hepatic tissues or cell types. Thus, in some embodiments, the lentiviral vector can comprise an endothelial specific promoter which would control expression of the FIX transgene in hepatic endothelial tissue, or a hepatocyte specific promoter which would control expression of the FIX transgene in hepatocytes, or both.

In some embodiments, the lentiviral vector comprises a tissue-specific promoter or tissue-specific promoters that control the expression of the FIX transgene in tissues other than liver. In some embodiments, the isolated nucleic acid molecule is stably integrated into the genome of the target cell or target tissue, for example, in the genome of a hepatocyte or in the genome of a hepatic endothelial cell.

In some embodiments, the lentiviral vector comprises at least one splice donor site. In some embodiments, the lentiviral vector comprises at least one splice acceptor site.

In some embodiments, the lentiviral vector comprises a gag sequence, a pol sequence, a rev sequence, a rev responsive element (RRE), or any combination thereof. In certain embodiments, the lentiviral vector comprises a full-length gag sequence. In some embodiments, the lentiviral vector comprises a truncated gag sequence. In certain embodiments, the lentiviral vector comprises a full-length pol sequence. In some embodiments, the lentiviral vector comprises a truncated pol sequence. In certain embodiments, the lentiviral vector comprises a full-length rev sequence. In some embodiments, the lentiviral vector comprises a truncated rev sequence. In certain embodiments, the lentiviral vector comprises a full-length RRE sequence. In some embodiments, the lentiviral vector comprises a truncated RRE sequence.

In some embodiments, the lentiviral vector comprises an enhancer, a promoter, a target sequence for a microRNA (miRNA), a post-transcriptional regulatory element, a packaging signal, a poly-A sequence, an intron sequence, or any combination thereof. In certain embodiments, the lentiviral vector comprises an enhancer that promotes the expression of the nucleotide sequence in a liver cell.

In certain embodiments, it will be useful to include within the lentiviral vector one or more miRNA target sequences which, for example, are operably linked to the FIX transgene. Thus, the disclosure also provides at least one miRNA sequence target operably linked to the FIX nucleotide sequence or otherwise inserted within a lentiviral vector. More than one copy of a miRNA target sequence included in the lentiviral vector can increase the effectiveness of the system.

Also included are different miRNA target sequences. For example, lentiviral vectors which express more than one transgene can have the transgene under control of more than one miRNA target sequence, which can be the same or different. The miRNA target sequences can be in tandem, but other arrangements are also included. The transgene expression cassette, containing miRNA target sequences, can also be inserted within the lentiviral vector in antisense orientation. Antisense orientation can be useful in the production of viral particles to avoid expression of gene products which can otherwise be toxic to the producer cells.

In other embodiments, the lentiviral vector comprises 1, 2, 3, 4, 5, 6, 7 or 8 copies of the same or different miRNA target sequence. However, in certain other embodiments, the lentiviral vector will not include any miRNA target sequence. Choice of whether or not to include an miRNA target sequence (and how many) will be guided by parameters such as the intended tissue target, the level of expression required, etc.

In one embodiment, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In another embodiment, the target sequence is an miR142 target (tccataaagt aggaaacact aca (SEQ ID NO: 27)). In one embodiment, the lentiviral vector comprises at least one, at least two, at least three, at least four, at least five, or at least six copies of miR-142 target sequences. In some embodiments, the lentiviral vector comprises four copies of miR-142 target sequences. In certain embodiments, the complementary sequence of hematopoietic-specific microRNAs, such as miR-142 (142T) or “142-3pT”, is incorporated into the 3′ untranslated region of a lentiviral vector, making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can impose a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

In other embodiments, the nucleotide sequence encoding a polypeptide with FIX activity in the lentiviral vector of the present disclosure comprises, consists, or consist essentially of a lentiviral vector comprising coFIX-1-R338L (SEQ ID NO: 1).

### C.3. Lentiviral Vectors

Lentiviruses include members of the bovine lentivirus group, equine lentivirus group, feline lentivirus group, ovinecaprine lentivirus group, and primate lentivirus group. The development of lentiviral vectors for gene therapy has been reviewed in Klimatcheva et al. (1999) Frontiers in Bioscience 4:481-496. The design and use of lentiviral vectors suitable for gene therapy is described for example in U.S. Pat. Nos. 6,207,455 and 6,615,782. Examples of lentivirus include, but are not limited to, HIV-1, HIV-2, HIV-1/HIV-2 pseudotype, HIV-1/SIV, FIV, caprine arthritis encephalitis virus (CAEV), equine infectious anemia virus, and bovine immunodeficiency virus.

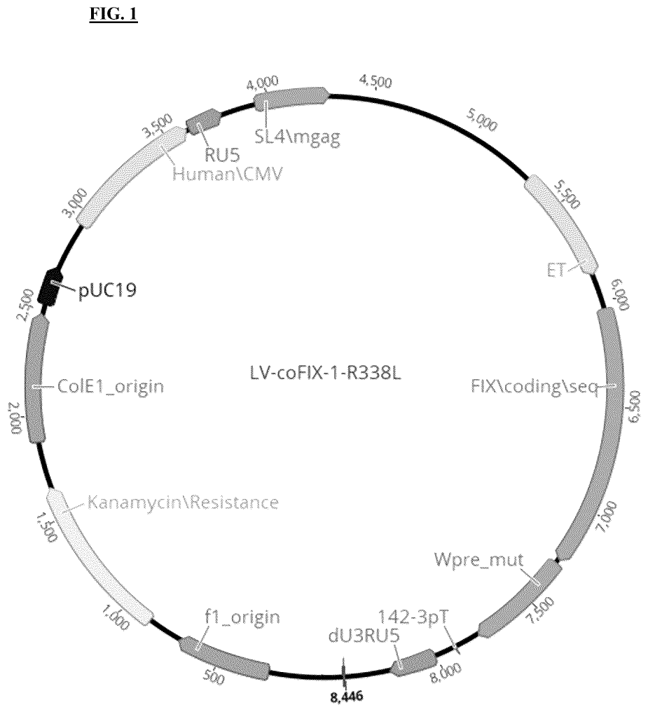

A schematic representation of a lentiviral vector of the present disclosure is presented in FIG. 1. In some embodiments, the lentiviral vector of the present disclosure is “third-generation” lentiviral vector. As used herein, the term “third-generation” lentiviral vector refers to a lentiviral packaging system that has the characteristics of a second-generation vector system, and that further lacks a functional tat gene, such as one from which the tat gene has been deleted or inactivated. Typically, the gene encoding rev is provided on a separate expression construct. See, e.g., Dull et al. (1998) J. Virol. 72: 8463-8471. As used herein, a “second-generation” lentiviral vector system refers to a lentiviral packaging system that lacks functional accessory genes, such as one from which the accessory genes vif, vpr, vpu, and nef have been deleted or inactivated. See, e.g., Zufferey et al. (1997) Nat. Biotechnol. 15:871-875. As used herein, “packaging system” refers to a set of viral constructs comprising genes that encode viral proteins involved in packaging a recombinant virus. Typically, the constructs of the packaging system will ultimately be incorporated into a packaging cell.

In some embodiments, the third-generation lentiviral vector of the present disclosure is a self-inactivating lentiviral vector. In some embodiments, the lentiviral vector is a VSV.G pseudo type lentiviral vector. In some embodiments, the lentiviral vector comprises a mammalian-specific promoter for transgene expression. In some embodiments, the mammalian-specific promoter is a cytomegalovirus (CMV) promoter. In some embodiments, the lentiviral vector comprises a hepatocyte-specific promoter for transgene expression. In some embodiments, the hepatocyte-specific promoter is an enhanced transthyretin promoter. In some embodiments, the lentiviral vector comprises one or more target sequences for miR-142 to reduce immune response to the transgene product. In some embodiments, incorporating one or more target sequences for miR-142 into a lentiviral vector of the present disclosure allows for a desired transgene expression profile. For example, incorporating one or more target sequences for miR-142 may suppress transgene expression in intravascular and extravascular hematopoietic lineages, whereas transgene expression is maintained in nonhematopoietic cells. No oncogenesis has been detected in tumor prone mice treated with the lentiviral vector system of the present disclosure. See Brown et al. (2007) Blood 110:4144-52, Brown at al. (2006) Nat. Ned. 12:585-91, and Cantore et al. (2015) Sci. Transl. Med. 7(277):277ra28.

Lentiviral vectors of the disclosure include polynucleotides encoding the polypeptides having FIX activity described herein. In one embodiment, the polypeptide having FIX activity is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

In certain embodiments, the lentiviral vector is a vector of a recombinant lentivirus capable of transducing non-dividing cells. In certain embodiments, the lentiviral vector is a vector of a recombinant lentivirus capable of transducing liver cells (e.g., hepatocytes). The lentivirus genome and the proviral DNA typically have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5 and 3′ LTR's serve to promote transcription and polyadenylation of the virion RNA's. The LTR contains all other cis-acting sequences necessary for viral replication. Lentiviruses have additional genes including vif, vpr, tat, rev, vpu, nef and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA.

However, the resulting mutant remains capable of directing the synthesis of all virion proteins. The disclosure provides a method of producing a recombinant lentiviral vector capable of transducing a non-dividing cell comprising transfecting a suitable host cell with two or more vectors carrying the packaging functions, namely gag, pol and env, as well as rev and tat. As will be disclosed herein below, vectors lacking a functional tat gene are desirable for certain applications. Thus, for example, a first vector can provide a nucleic acid encoding a viral gag and a viral pol and another vector can provide a nucleic acid encoding a viral env to produce a packaging cell. Introducing a vector providing a heterologous gene, herein identified as a transfer vector, into that packaging cell yields a producer cell which releases infectious viral particles carrying the foreign gene of interest.

According to the above-indicated configuration of vectors and foreign genes, the second vector can provide a nucleic acid encoding a viral envelope (env) gene. The env gene can be derived from nearly any suitable virus, including retroviruses. In some embodiments, the env protein is an amphotropic envelope protein which allows transduction of cells of human and other species.

Examples of retroviral-derived env genes include, but are not limited to: Moloney murine leukemia virus (MoMuLV or MMLV), Harvey murine sarcoma virus (HaMuSV or HSV), murine mammary tumor virus (MuMTV or MMTV), gibbon ape leukemia virus (GaLV or GALV), human immunodeficiency virus (HIV) and Rous sarcoma virus (RSV). Other env genes such as Vesicular stomatitis virus (VSV) protein G (VSV G), that of hepatitis viruses and of influenza also can be used. In some embodiments, the viral env nucleic acid sequence is associated operably with regulatory sequences described elsewhere herein.

In certain embodiments, the lentiviral vector has the HIV virulence genes env, vif, vpr, vpu and nef deleted without compromising the ability of the vector to transduce non-dividing cells. In some embodiments, the lentiviral vector comprises a deletion of the U3 region of the 3′ LTR. The deletion of the U3 region can be the complete deletion or a partial deletion.

In some embodiments, the lentiviral vector of the disclosure comprising the polypeptide having FIX activity nucleotide sequence described herein can be transfected in a cell with (a) a first nucleotide sequence comprising a gag, a pol, or gag and pol genes and (b) a second nucleotide sequence comprising a heterologous env gene; wherein the lentiviral vector lacks a functional tat gene. In other embodiments, the cell is further transfected with a fourth nucleotide sequence comprising a rev gene. In certain embodiments, the lentiviral vector lacks functional genes selected from vif, vpr, vpu, vpx and nef, or a combination thereof.

In certain embodiments, a lentiviral vector of the instant disclosure comprises one or more nucleotide sequences encoding a gag protein, a Rev-response element, a central polypurine track (cPPT), or any combination thereof.

In some embodiments, the lentiviral vector contains on its surface one or more polypeptides that improve the targeting and/or activity of the lentiviral vector or the encoded polypeptide having FIX activity. The one or more polypeptides can be incorporated during budding of the lentiviral vector from a host cell. During lentiviral production, viral particles bud off from a producing host cell. During the budding process, the viral particle takes on a lipid coat, which is derived from the lipid membrane of the host cell. As a result, the lipid coat of the viral particle can include membrane bound polypeptides that were previously present on the surface of the host cell.

In some embodiments, the lentiviral vector expresses one or more polypeptides on its surface that inhibit an immune response to the lentiviral vector following administration to a human subject. In some embodiments, the surface of the lentiviral vector comprises one or more CD47 molecules. CD47 is a “marker of self” protein, which is ubiquitously expressed on human cells. Surface expression of CD47 inhibits macrophage-induced phagocytosis of endogenous cells through the interaction of CD47 and macrophage expressed-SIRPα. Cells expressing high levels of CD47 are less likely to be targeted and destroyed by human macrophages in vivo.

In some embodiments, the lentiviral vector comprises a high concentration of CD47 polypeptide molecules on its surface. In some embodiments, the lentiviral vector is produced in a cell line that has a high expression level of CD47. In certain embodiments, the lentiviral vector is produced in a CD47high cell, wherein the cell has high expression of CD47 on the cell membrane. In particular embodiments, the lentiviral vector is produced in a CD47high HEK 293T cell, wherein the HEK 293T is has high expression of CD47 on the cell membrane. In some embodiments, the HEK 293T cell is modified to have increased expression of CD47 relative to unmodified HEK 293T cells. In certain embodiments, the CD47 is human CD47.

In some embodiments, the lentiviral vector comprises a human CD47 which comprises an amino acid sequence at least about 60%, at least about 70%, at least about 80%, at least about 85%, at least about 90%, at least about 95%, at least about 96%, at least about 97%, at least about 98%, at least about 99%, or about 100% identical to the amino acid sequence set forth in SEQ ID NO: 14.

In some embodiments, the lentiviral vector has little or no surface expression of major histocompatibility complex class I (MHC-I). Surface expressed MHC-I displays peptide fragments of “non-self” proteins from within a cell, such as protein fragments indicative of an infection, facilitating an immune response against the cell. In some embodiments, the lentiviral vector is produced in a MHC-Ilow cell, wherein the cell has reduced expression of MHC-I on the cell membrane. In some embodiments, the lentiviral vector is produced in an MHC-I− (or “MHC-I^{free}”, “MHC-1^{neg}” or “MHC-negative”) cell, wherein the cell lacks expression of MHC-I.

In particular embodiments, the lentiviral vector comprises a lipid coat comprising a high concentration of CD47 polypeptides and lacking MHC-I polypeptides. In certain embodiments, the lentiviral vector is produced in a CD47^{high}/MHC-I^{low} cell line, e.g., a CD47^{high}/MHC-I^{low} HEK 293T cell line. In some embodiments, the lentiviral vector is produced in a CD47^{high}/MHC-I^{free} cell line, e.g., a CD47^{high}/MHC-I^{free} HEK 293T cell line. Examples of lentiviral vectors are disclosed in U.S. Pat. No. 9,050,269 and International Publication Nos. WO9931251, WO9712622, WO9817815, WO9817816, and WO9818934, which are incorporated herein by reference in their entireties.

### III. Pharmaceutical Compositions

Compositions containing a lentiviral vector, a nucleic acid molecule, a polypeptide encoded by the nucleic acid molecule, or a host cell of the present disclosure can contain a suitable pharmaceutically acceptable carrier. For example, they can contain excipients and/or auxiliaries that facilitate processing of the active compounds into preparations designed for delivery to the site of action.

In one embodiment, the present disclosure is directed to a pharmaceutical composition comprising (a) a nucleic acid molecule, a lentiviral vector, a polypeptide, or a host cell disclosed herein; and (b) a pharmaceutically acceptable excipient.

The pharmaceutical composition can be formulated for parenteral administration (i.e. intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

In one embodiment, the route of administration of the lentiviral vectors is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the disclosure, a form for administration would be a solution for injection, in particular for intravenous or intraarterial injection or drip. Usually, a suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, the lentiviral vector can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to, clotting disorders.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

The pharmaceutical composition can also be formulated for rectal administration as a suppository or retention enema, e.g., containing conventional suppository bases such as cocoa butter or other glycerides.

Supplementary active compounds can be incorporated into the compositions. In one embodiment, the nucleic acid molecule of the disclosure is formulated with a clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens can be adjusted to provide the optimum desired response. For example, a single bolus can be administered, several divided doses can be administered over time, or the dose can be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

Doses intermediate in the above ranges are also intended to be within the scope of the disclosure. Subjects can be administered such doses daily, on alternative days, weekly or according to any other schedule determined by empirical analysis. An exemplary treatment entails administration in multiple dosages over a prolonged period, for example, of at least six months.

The lentiviral vector of the disclosure may be administered to a subject at different developmental stages. For example, in humans, different developmental stages may be classified as neonate (e.g., under 1 month of age), infant (1 month to 2 years of age), child (2 years to 12 years of age), adolescent (12 years to under 16 years of age), or adult (over 16 years of age). In some embodiments, the lentiviral vector of the disclosure is administered to a human neonate. In some embodiments, the lentiviral vector of the disclosure is administered to a human subject under about 1 month of age. In some embodiments, the lentiviral vector of the disclosure is administered to a human infant. In some embodiments, the lentiviral vector of the disclosure is administered to a human subject under between about 1 month to about 2 years of age. In some embodiments, the lentiviral vector of the disclosure is administered to a human child. In some embodiments, the lentiviral vector of the disclosure is administered to a human subject between about 2 years to about 12 years of age. In some embodiments, the lentiviral vector of the disclosure is administered to a human adolescent. In some embodiments, the lentiviral vector of the disclosure is administered to a human subject between about 12 years to about 16 years of age. In some embodiments, the lentiviral vector of the disclosure is administered to a human adult. In some embodiments, the lentiviral vector of the disclosure is administered to a human subject greater than about 16 years of age. One of skill in the art would be able to determine the developmental stage in other organisms. For example, one of skill in the art would understand that a two-week old mouse is an adolescent.

Dosage and frequency of the lentiviral vectors of the disclosure may vary depending on various factors known to those of skill in the art.

The lentiviral vector of the disclosure can optionally be administered in combination with other agents that are effective in treating the disorder or condition in need of treatment (e.g., prophylactic or therapeutic).

As used herein, the administration of lentiviral vectors of the disclosure in conjunction or combination with an adjunct therapy means the sequential, simultaneous, coextensive, concurrent, concomitant or contemporaneous administration or application of the therapy and the disclosed polypeptides. Those skilled in the art will appreciate that the administration or application of the various components of the combined therapeutic regimen can be timed to enhance the overall effectiveness of the treatment. A skilled artisan (e.g., a physician) would be readily be able to discern effective combined therapeutic regimens without undue experimentation based on the selected adjunct therapy and the teachings of the instant specification.

It will further be appreciated that the lentiviral vectors of the disclosure can be used in conjunction or combination with an agent or agents (e.g., to provide a combined therapeutic regimen). Exemplary agents with which a lentiviral vector of the instant disclosure can be combined include agents that represent the current standard of care for a particular disorder being treated. Such agents can be chemical or biologic in nature. The term “biologic” or “biologic agent” refers to any pharmaceutically active agent made from living organisms and/or their products which is intended for use as a therapeutic.

The amount of agent to be used in combination with the lentiviral vectors of the instant disclosure can vary by subject or can be administered according to what is known in the art. See, e.g., Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9th ed. 1996). In another embodiment, an amount of such an agent consistent with the standard of care is administered.

In certain embodiments, the lentiviral vectors of the present disclosure are administered in conjunction with an immunosuppressive, anti-allergic, or anti-inflammatory agent. These agents generally refer to substances that act to suppress or mask the immune system of the subject being treated herein. These agents include substances that suppress cytokine production, downregulate or suppress self-antigen expression, or mask the MHC antigens. Examples of such agents include 2-amino-6-aryl-5-substituted pyrimidines; azathioprine; cyclophosphamide; bromocryptine; danazol; dapsone; glutaraldehyde; anti-idiotypic antibodies for MHC antigens and MHC fragments; cyclosporin A; steroids such as glucocorticosteroids, e.g., prednisone, methylprednisolone, and dexamethasone; cytokine or cytokine receptor antagonists including anti-interferon-γ, -β, or -α antibodies, anti-tumor necrosis factor-α antibodies, anti-tumor necrosis factor-3 antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies; soluble peptide containing a LFA-3 binding domain; streptokinase; TGF-β; streptodornase; FK506; RS-61443; deoxyspergualin; and rapamycin. In certain embodiments, the agent is an antihistamine. An “antihistamine” as used herein is an agent that antagonizes the physiological effect of histamine. Examples of antihistamines are chlorpheniramine, diphenhydramine, promethazine, cromolyn sodium, astemizole, azatadine maleate, bropheniramine maleate, carbinoxamine maleate, cetirizine hydrochloride, clemastine fumarate, cyproheptadine hydrochloride, dexbrompheniramine maleate, dexchlorpheniramine maleate, dimenhydrinate, diphenhydramine hydrochloride, doxylamine succinate, fexofendadine hydrochloride, terphenadine hydrochloride, hydroxyzine hydrochloride, loratidine, meclizine hydrochloride, tripelannamine citrate, tripelennamine hydrochloride, and triprolidine hydrochloride.

Immunosuppressive, anti-allergic, or anti-inflammatory agents may be incorporated into the lentiviral vector administration regimen. For example, administration of immunosuppressive or anti-inflammatory agents may commence prior to administration of the disclosed lentiviral vectors and may continue with one or more doses thereafter. In certain embodiments, the immunosuppressive or anti-inflammatory agents are administered as premedication to the lentiviral vectors.

As previously discussed, the lentiviral vectors of the present disclosure, can be administered in a pharmaceutically effective amount for the in vivo treatment of clotting disorders. In this regard, it will be appreciated that the lentiviral vectors of the disclosure can be formulated to facilitate administration and promote stability of the active agent. Preferably, pharmaceutical compositions in accordance with the present disclosure comprise a pharmaceutically acceptable, non-toxic, sterile carrier such as physiological saline, non-toxic buffers, preservatives and the like. Of course, the pharmaceutical compositions of the present disclosure can be administered in single or multiple doses to provide for a pharmaceutically effective amount of the polypeptide.

In addition to the active compound, the liquid dosage form can contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

In some embodiments, the composition is administered by a route selected from the group consisting of topical administration, intraocular administration, intrathecal administration, and subdural administration. The parenteral administration can be intravenous or subcutaneous administration.

In some embodiments, the composition is used to treat a bleeding disease or condition in a subject in need thereof. The bleeding disease or condition is selected from the group consisting of a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath and any combinations thereof. In still other embodiments, the subject is scheduled to undergo a surgery. In yet other embodiments, the treatment is prophylactic or on-demand.

A number of tests are available to assess the function of the coagulation system: activated partial thromboplastin time (aPTT) test, chromogenic assay, ROTEM® assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM® analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

### IV. Nucleic Acid Molecules

The disclosure also provides isolated nucleic acid molecules encoding a polypeptide having FIX activity. In certain embodiments, the isolated acid molecule comprises a nucleic acid sequence that has at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1.

In some embodiments, the isolated acid molecule comprises a nucleotide sequence that has at least about 85% sequence identity to the nucleotide sequence set forth in SEQ ID NO: 1. In some embodiments, the nucleotide sequence has at least about 90% sequence identity to the nucleotide sequence set forth in SEQ ID NO: 1. In some embodiments, the nucleotide sequence has at least about 91% sequence identity to the nucleotide sequence set forth in SEQ ID NO: 1. In some embodiments, the nucleotide sequence has at least about 92% sequence identity to the nucleotide sequence set forth in SEQ ID NO: 1. In some embodiments, the nucleotide sequence has at least about 93% sequence identity to the nucleotide sequence set forth in SEQ ID NO: 1. In some embodiments, the nucleotide sequence has at least about 94% sequence identity to the nucleotide sequence set forth in SEQ ID NO: 1. In some embodiments, the nucleotide sequence has at least about 95% sequence identity to the nucleotide sequence set forth in SEQ ID NO: 1. In some embodiments, the nucleotide sequence has at least about 96% sequence identity to the nucleotide sequence set forth in SEQ ID NO: 1. In some embodiments, the nucleotide sequence has at least about 97% sequence identity to the nucleotide sequence set forth in SEQ ID NO: 1. In some embodiments, the nucleotide sequence has at least about 98% sequence identity to the nucleotide sequence set forth in SEQ ID NO: 1. In some embodiments, the nucleotide sequence has at least about 99% sequence identity to the nucleotide sequence set forth in SEQ ID NO: 1. In some embodiments, the nucleotide sequence is identical to the nucleotide sequence set forth in SEQ ID NO: 1.

In certain embodiments, the isolated acid molecule further comprises a nucleic acid sequence encoding a signal peptide. In some embodiments, the nucleic acid sequence encoding a signal peptide has at least 60%, at least 70%, at least 80%, at least 90%, at least 95%, at least 96%, at least 97%, at least 98%, at least 99%, or 100% sequence identity to: (i) nucleotides 1-84 of SEQ ID NO: 2; (ii) nucleotides 1-84 of SEQ ID NO: 3; (iii) nucleotides 1-84 of SEQ ID NO: 4; (iv) nucleotides 1-84 of SEQ ID NO: 5; (v) nucleotides 1-84 of SEQ ID NO: 6; or (vi) nucleotides 1-84 of SEQ ID NO: 7. In some embodiments, the nucleic acid sequence encoding a signal peptide comprises the nucleotide sequence set forth in (i) nucleotides 1-84 of SEQ ID NO: 2; (ii) nucleotides 1-84 of SEQ ID NO: 3; (iii) nucleotides 1-84 of SEQ ID NO: 4; (iv) nucleotides 1-84 of SEQ ID NO: 5; (v) nucleotides 1-84 of SEQ ID NO: 6; or (vi) nucleotides 1-84 of SEQ ID NO: 7.

In certain embodiments, the isolated acid molecule further comprises a nucleic acid sequence encoding a propeptide. In some embodiments, the nucleic acid sequence encoding a propeptide has at least 60%, at least 70%, at least 80%, at least 90%, at least 95%, at least 96%, at least 97%, at least 98%, at least 99%, or 100% sequence identity to: (i) nucleotides 85-138 of SEQ ID NO: 2; (ii) nucleotides 85-138 of SEQ ID NO: 3; (iii) nucleotides 85-138 of SEQ ID NO: 4; (iv) nucleotides 85-138 of SEQ ID NO: 5; (v) nucleotides 85-138 of SEQ ID NO: 6; or (vi) nucleotides 85-138 of SEQ ID NO: 7. In some embodiments, the nucleic acid sequence encoding a propeptide comprises the nucleotide sequence set forth in (i) nucleotides 85-138 of SEQ ID NO: 2; (ii) nucleotides 85-138 of SEQ ID NO: 3; (iii) nucleotides 85-138 of SEQ ID NO: 4; (iv) nucleotides 85-138 of SEQ ID NO: 5; (v) nucleotides 85-138 of SEQ ID NO: 6; or (vi) nucleotides 85-138 of SEQ ID NO: 7.

The disclosure also provides vectors comprising a nucleic acid molecule described herein. In some embodiments, the vector is a lentiviral vector, e.g., any lentiviral vector disclosed herein. In certain embodiments, the vector comprises a nucleic acid sequence that has at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

In some embodiments, the vector comprises a nucleotide sequence that has at least about 70%, at least about 75%, at least about 80%, at least about 85%, at least about 90%, at least about 95%, at least about 96%, at least about 97%, at least about 98%, at least about 99%, or about 100% sequence identity to the nucleotide sequence set forth in SEQ ID NO: 1. In some embodiments, the vector comprises a nucleotide sequence that has at least about 85% sequence identity to the nucleotide sequence set forth in SEQ ID NO: 1. In some embodiments, the vector comprises a nucleotide sequence that has at least about 90% sequence identity to the nucleotide sequence set forth in SEQ ID NO: 1. In some embodiments, the vector comprises a nucleotide sequence that has at least about 91% sequence identity to the nucleotide sequence set forth in SEQ ID NO: 1. In some embodiments, the vector comprises a nucleotide sequence that has at least about 92% sequence identity to the nucleotide sequence set forth in SEQ ID NO: 1. In some embodiments, the vector comprises a nucleotide sequence that has at least about 93% sequence identity to the nucleotide sequence set forth in SEQ ID NO: 1. In some embodiments, the vector comprises a nucleotide sequence that has at least about 94% sequence identity to the nucleotide sequence set forth in SEQ ID NO: 1. In some embodiments, the vector comprises a nucleotide sequence that has at least about 95% sequence identity to the nucleotide sequence set forth in SEQ ID NO: 1. In some embodiments, the vector comprises a nucleotide sequence that has at least about 96% sequence identity to the nucleotide sequence set forth in SEQ ID NO: 1. In some embodiments, the vector comprises a nucleotide sequence that has at least about 97% sequence identity to the nucleotide sequence set forth in SEQ ID NO: 1. In some embodiments, the vector comprises a nucleotide sequence that has at least about 98% sequence identity to the nucleotide sequence set forth in SEQ ID NO: 1. In some embodiments, the vector comprises a nucleotide sequence that has at least about 99% sequence identity to the nucleotide sequence set forth in SEQ ID NO: 1. In some embodiments, the vector comprises a nucleotide sequence that is identical to the nucleotide sequence set forth in SEQ ID NO: 1.

In some embodiments, the vector further comprises one or more regulatory element described herein. In certain embodiments, the vector comprises a tissue specific promoter. In certain embodiments, the tissue specific promoter selectively enhances expression of the polypeptide with FIX activity in a target liver cell. In certain embodiments, the tissue specific promoter that selectively enhances expression of the polypeptide with FIX activity in a target liver cell comprises an APOA2 promoter, SERPINA1 (hAAT) promoter, mTTR promoter, MIR122 promoter, or any combination thereof. In some embodiments, the target liver cell is a hepatocyte.

### V. Tissue Specific Expression

In certain embodiments, it will be useful to include within the lentiviral vector one or more miRNA target sequences which, for example, are operably linked to the optimized FIX transgene. Thus, the disclosure also provides at least one miRNA sequence target operably linked to the optimized FIX nucleotide sequence or otherwise inserted within a lentiviral vector. More than one copy of a miRNA target sequence included in the lentiviral vector can increase the effectiveness of the system.

Also included are different miRNA target sequences. For example, lentiviral vectors which express more than one transgene can have the transgene under control of more than one miRNA target sequence, which can be the same or different. The miRNA target sequences can be in tandem, but other arrangements are also included. The transgene expression cassette, containing miRNA target sequences, can also be inserted within the lentiviral vector in antisense orientation. Antisense orientation can be useful in the production of viral particles to avoid expression of gene products which can otherwise be toxic to the producer cells.

In other embodiments, the lentiviral vector comprises 1, 2, 3, 4, 5, 6, 7 or 8 copies of the same or different miRNA target sequence. However in certain other embodiments, the lentiviral vector will not include any miRNA target sequence. Choice of whether or not to include an miRNA target sequence (and how many) will be guided by known parameters such as the intended tissue target, the level of expression required, etc.

In one embodiment, the target sequence is an miR-223 target which has been reported to block expression most effectively in myeloid committed progenitors and at least partially in the more primitive HSPC. miR-223 target can block expression in differentiated myeloid cells including granulocytes, monocytes, macrophages, myeloid dendritic cells. miR-223 target can also be suitable for gene therapy applications relying on robust transgene expression in the lymphoid or erythroid lineage. miR-223 target can also block expression very effectively in human HSC.

In another embodiment, the target sequence is an miR142 target (e.g., tccataaagt aggaaacact aca (SEQ ID NO: 27)). In one embodiment, the lentiviral vector comprises 4 copies of miR-142 target sequences. In certain embodiments, the complementary sequence of hematopoietic-specific microRNAs, such as miR-142 (142T), is incorporated into the 3′ untranslated region of a lentiviral vector, making the transgene-encoding transcript susceptible to miRNA-mediated down-regulation. By this method, transgene expression can be prevented in hematopoietic-lineage antigen presenting cells (APC), while being maintained in non-hematopoietic cells (Brown et al., Nat Med 2006). This strategy can impose a stringent post-transcriptional control on transgene expression and thus enables stable delivery and long-term expression of transgenes. In some embodiments, miR-142 regulation prevents immune-mediated clearance of transduced cells and/or induce antigen-specific Regulatory T cells (T regs) and mediate robust immunological tolerance to the transgene-encoded antigen.

In some embodiments, the target sequence is an miR181 target. Chen C-Z and Lodish H, Seminars in Immunology (2005) 17(2):155-165 discloses miR-181, a miRNA specifically expressed in B cells within mouse bone marrow (Chen and Lodish, 2005). It also discloses that some human miRNAs are linked to leukemias.

The target sequence can be fully or partially complementary to the miRNA. The term “fully complementary” means that the target sequence has a nucleic acid sequence which is 100% complementary to the sequence of the miRNA which recognizes it. The term “partially complementary” means that the target sequence is only in part complementary to the sequence of the miRNA which recognizes it, whereby the partially complementary sequence is still recognized by the miRNA. In other words, a partially complementary target sequence in the context of the present disclosure is effective in recognizing the corresponding miRNA and effecting prevention or reduction of transgene expression in cells expressing that miRNA. Examples of the miRNA target sequences are described at WO2007/000668, WO2004/094642, WO2010/055413, or WO2010/125471, which are incorporated herein by reference in their entireties.

### VI. Host Cells and Methods of Making

The disclosure also provides a host cell comprising a nucleic acid molecule or lentiviral vector of the disclosure. Certain aspects of the disclosure are directed to making or producing lentiviral vectors comprising transfecting and/or transforming a host cell with a lentiviral vector disclosed herein. As used herein, the term “transformation” shall be used in a broad sense to refer to the introduction of DNA into a recipient host cell that changes the genotype and consequently results in a change in the recipient cell.

“Host cells” refers to cells that have been transformed with a lentiviral vector disclosed herein. The host cells of the present disclosure are preferably of mammalian origin; most preferably of human or mouse origin. Those skilled in the art are credited with ability to preferentially determine particular host cell lines which are best suited for their purpose. Exemplary host cell lines include, but are not limited to, CHO, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte), PER.C6®, NS0, CAP, BHK21, and HEK 293 (human kidney). In one particular embodiment, the host cell is selected from the group consisting of: a CHO cell, a HEK293 cell (e.g., a HEK293T cell), a BHK21 cell, a PER.C6® cell, a NS0 cell, a CAP cell and any combination thereof. In some embodiments, the host cells of the present disclosure are of insect origin. In one particular embodiment, the host cells are SF9 cells. Host cell lines are typically available from commercial services, the American Tissue Culture Collection, or from published literature.

Introduction of the nucleic acid molecules or vectors of the disclosure into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

Host cells comprising the isolated nucleic acid molecules or lentiviral vectors of the disclosure are grown in an appropriate growth medium. As used herein, the term “appropriate growth medium” means a medium containing nutrients required for the growth of cells. Nutrients required for cell growth can include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CDoptiCHO (Invitrogen, Carlsbad, CA.). In another embodiment, the medium is CD17 (Invitrogen, Carlsbad, CA.). Selection of a medium appropriate for the particular cell line used is within the level of those ordinary skilled in the art.

In some embodiments, the host cell is further modified as described herein. For example, the host cell can be modified to overexpress CD47, as described herein. In some embodiments, the host cell is modified to lack surface-exposed MHC-I. In some embodiments, the host cell is modified to have decreased surface-exposed MHC-I, relative to an unmodified host cell. In certain embodiments, the host cell is a CD47^{high}/MHC-I^{low} HEK 293T cell.

Certain aspects of the disclosure are directed to methods of producing a lentiviral vector comprising culturing a host cell described herein under suitable conditions and isolating the lentiviral vector. In certain aspects, the disclosure is directed to methods of producing a lentiviral vector disclosed herein, comprising culturing a host cell described herein under suitable conditions and isolating the lentiviral vector.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

Having generally described this disclosure, a further understanding can be obtained by reference to the examples provided herein. These examples are for purposes of illustration only and are not intended to be limiting.

### EXAMPLES

### Example 1: LV-coFIX-1-R338L Mediated Long Term FIX Expression and Dose Response in Adult HemB Mice

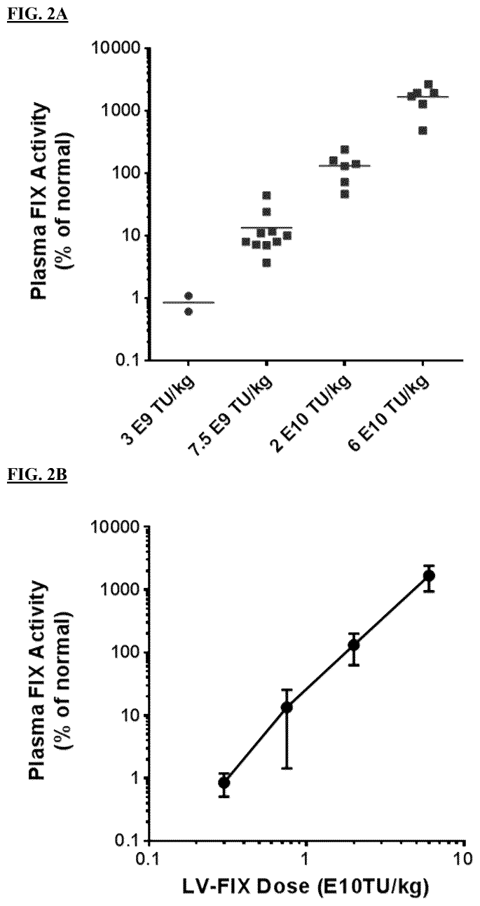

A codon optimized nucleotide sequence encoding a human FIX variant having a R338L (“Padua”) substitution (coFIX-1-R338L; SEQ ID NO: 1) was cloned into a lentiviral vector to create LV-coFIX-1-R338L (FIG. 1). To determine the dose response profile of LV-FIX in animal models, LV-coFIX-1-R338L generated in 293T cells was evaluated in adult HemB mouse. Eight-week old HemB mice were treated with LV-coFIX-1-R338L via tail vein injection at a dose of 3E9, 7.5E9, 2E10, or 6E10 TU/kg (n=2 to 10 animals/dose level). LV-FIX mediated plasma FIX activity and antigen level was monitored by FIX chromogenic and ELISA assay. The steady state FIX plasma level of each animal is shown in FIG. 2A, and the LV-coFIX-1-R338L dose response curve is shown in FIG. 2B. In a HemB mouse model, LV-coFIX-1-R338L has demonstrated a Log-Log dose response profile, and the LV-coFIX-1-R338L dose level required to achieve 10-200% of normal circulating FIX activity was determined to be between 5E9 to 2E10 TU/kg range in HemB mice.

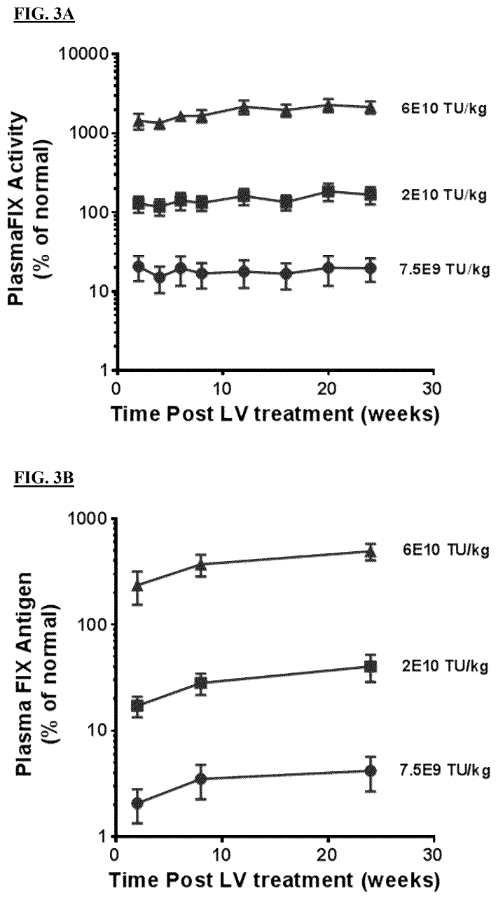

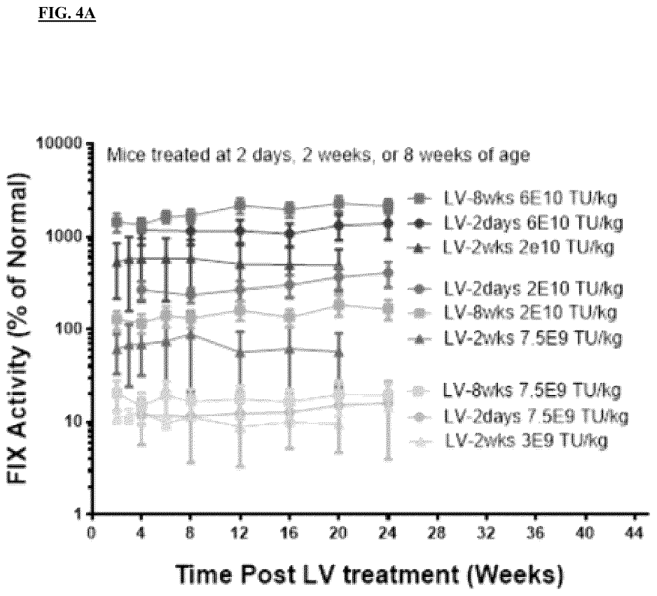

Long term FIX expression profile of LV-coFIX-1-R338L treated animals in the 3-higher dose level groups were monitored for 6-months post LV treatment. Circulating FIX activity (FIG. 3A) and antigen (FIG. 3B) levels were plotted. Consistent level of LV mediated FIX expression was observed in all experimental animals, no loss on FIX expression was detected within the duration of study which demonstrated the long-term stability of integrational gene therapy treatment. In addition, a lower percentage of normal FIX antigen level was observed (FIG. 3A) compared to that of FIX activity (FIG. 3B), which reflects the use of the gain of function R338L mutation in the FIX transgene.

### Example 2: LV-coFIX-1-R338L has Similar Transduction Efficiency in Adult and Neonatal Animals

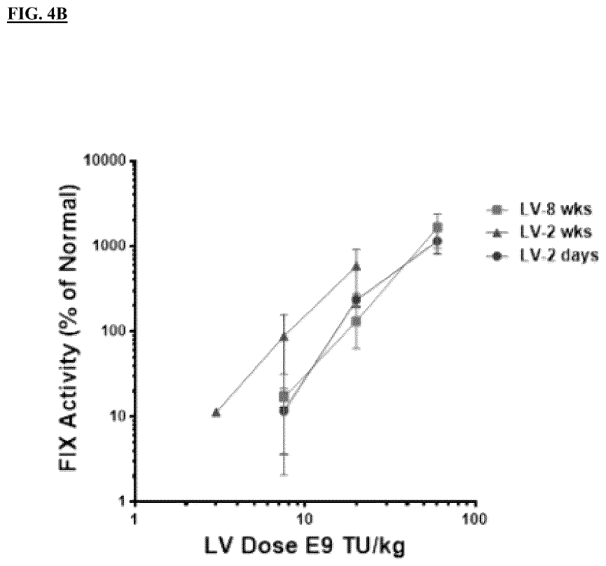

Lentiviral vector can integrate into the host genome to mediate long-lived transgene expression, so unlike the quick loss of AAV mediated transgene expression post neonatal treatment, lentiviral mediated transgene expression is expected to maintain a persistent transgene expression profile not only in adult animals but also in neonatal animals treated with LV-FIX. To assess the transduction efficiency and transgene expression profile of lentiviral FIX post neonatal treatment, two-day old HemB pups were treated with LV-coFIX-1-R338L via temporal vein injection at 7.5E9, 2E10 and 6E10 TU/kg. Compared to treatment at adult stage (administered at 8-weeks), systemically administered LV-coFIX-1-R338L mediated a persistent, similar level of FIX expression throughout the study period of six months at each dose level, suggesting that lentiviral FIX administration could effectively treat both adult and pediatric patients. Treatment of adolescent mice (administered at two-weeks) with LV-coFIX-1-R338L via temporal vein injection at 3E9, 7.5E9, or 2E10 TU/kg dose was also assessed. Levels of FIX expression at each dose level was higher than the comparable doses in the mice administered treatment at 8-weeks or two-days (n=6 animals/dose level/age; FIG. 4A). FIX activity was measured to determine dose response of LV-coFIX-1-R338L in HemB mice administered at 8-weeks and two-days via temporal vein injection at 7.5E9, 2E10, and 6E10 TU/kg dose, and two-weeks at 3E9, 7.5E9, or 2E10 TU/kg dose. Consistent with the long-range data in FIG. 4A, the mice administered treatment at two-weeks (adolescent mice) exhibited higher FIX activity compared to the mice administered treatment at two-days or eight-weeks (FIG. 4B).

### Example 3: Evaluation of CD47High LV-coFIX-1-R338L in Non-Human Primates

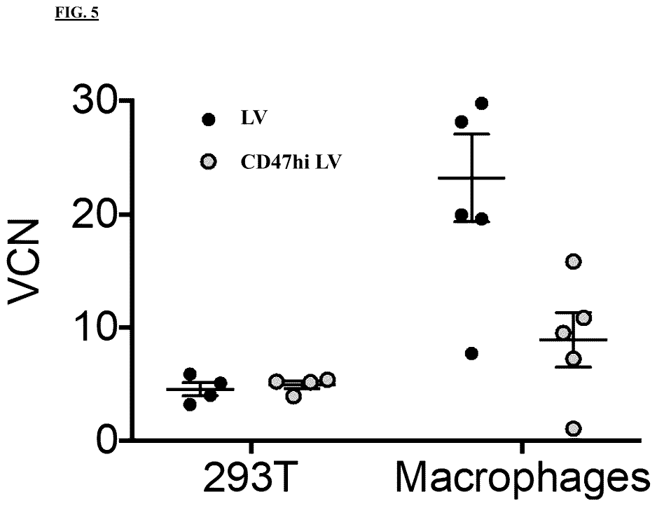

A human CD47 over expressing HEK293T cell line was generated to modulate the immune properties of lentiviral vectors. Lentiviral vector particles with a high surface level of human CD47 had shown lower Kupffer cell uptake and higher hepatocyte transduction in NOD mice (NOD mice can recognize human CD47). In addition, fewer lentiviral vector particles having high surface human CD47 expression were taken up by macrophages, relative to control lentiviral vectors not overexpressing CD47 (FIG. 5).

To further evaluate high surface level of human CD47 effect on in vivo liver transduction, CD47^{high} LV-coFIX-1-R338L was compared to LV-coFIX-1-R338L in non-human primates (NHP) post intravenous administration at 7.5E9 TU/kg dose, n=3/treatment group. Macaca nemestrina monkeys were used to avoid lentiviral vector restriction in NHPs post treatment.

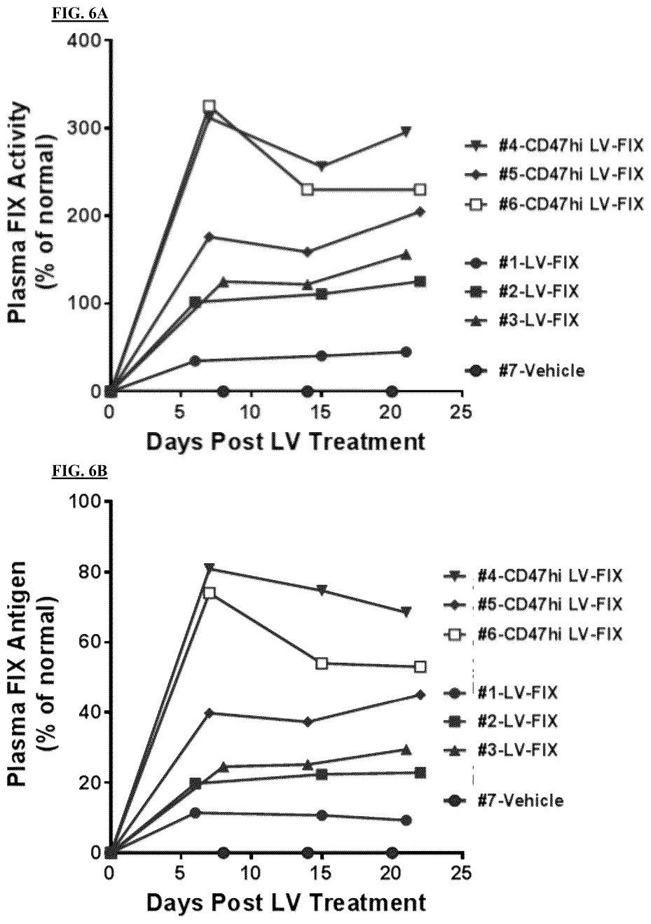

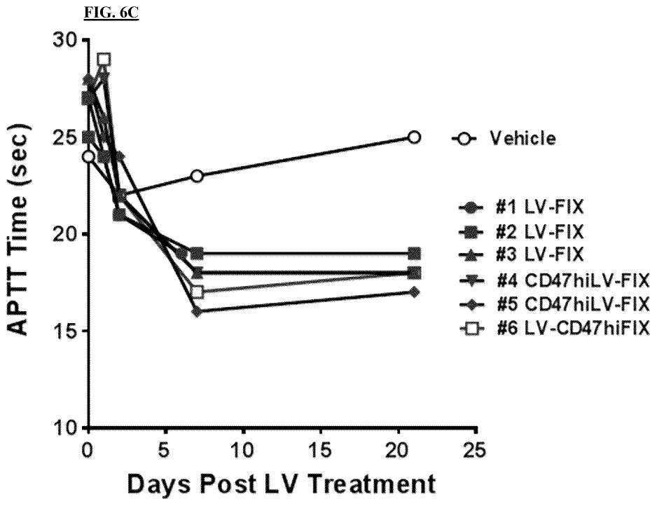

Circulating human FIX level post lentiviral vector treatment was measured by human FIX specific activity (FIG. 6A) and antigen assay (FIG. 6B). CD47^{high} LV-coFIX-1-R338L confers three-fold higher human FIX expression post lentiviral vector treatment compared to LV-coFIX-1-R338L, at 200-300% and 50-150% of normal FIX activity level, respectively (FIG. 6A). Use of CD47^{high} LV-coFIX-1-R338L could potentially lower LV-FIX and reduce the acute toxicity associated with lentiviral vector treatment.

In addition to human FIX expression levels, hemostasis of the treated animals was also monitored by APTT assay (FIG. 6C). While the APTT time of the vehicle treated animals remained in the same range, significantly shortened APTT time was observed for all LV-FIX treated animals (FIG. 6C), indicating that the human FIX protein resulted from lentiviral vector treatment is functionally active.

### Example 4: CD47High LV-coFIX-1-R338L Dose Response in Non-Human Primates

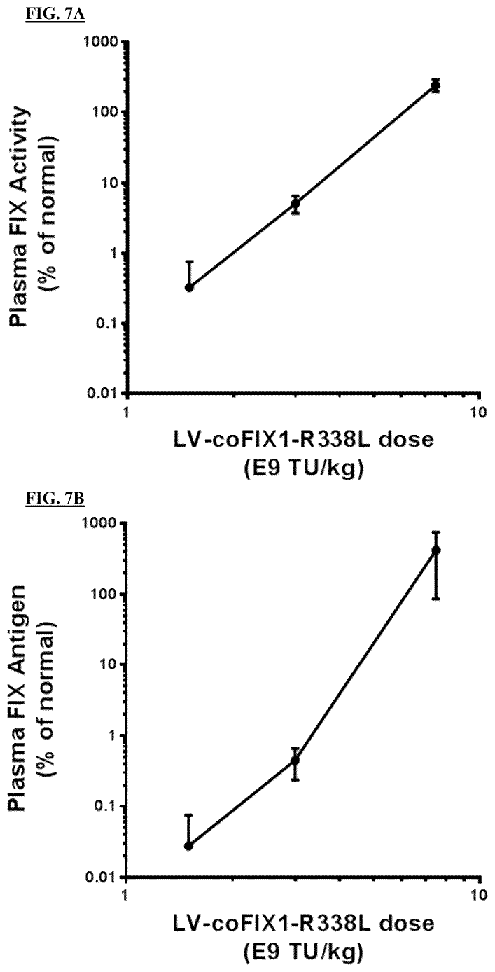

To determine the dose response profile of CD47^{high} LV-coFIX-1-R338L in NHPs, two lower doses of CD47^{high} LV-coFIX-1-R338L were tested at 1.5E9 and 3E9 TU/kg (n=3/dose level). Lentiviral vector-mediated human FIX expression was monitored by analyzing steady state circulated human FIX specific activity (FIG. 7A) and antigen level (FIG. 7B).

Consistent with the results observed in HemB mice, a log/log dose response curve was also observed for LV-coFIX-1-R338L in NHPs. The dose range of CD47^{high} LV-coFIX-1-R338L required to achieve 10-100% of normal circulating FIX level is between 3.5-6E9 TU/kg which is lower than that in HemB mice. The shift in the therapeutic dose range is due to the 5 to 10-fold higher human FIX expression level in NHPs compared to HemB mice at the same dose level, which could be attributed to the variation of animal species and recognition of human CD47 (human CD47 is not recognized in HemB mice).

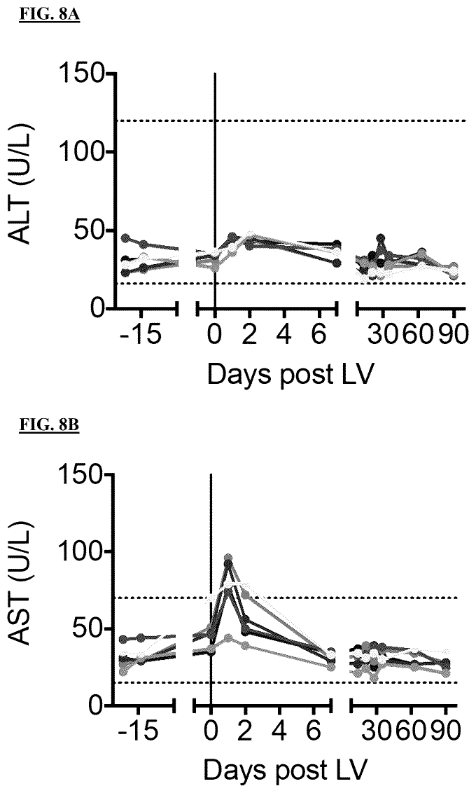

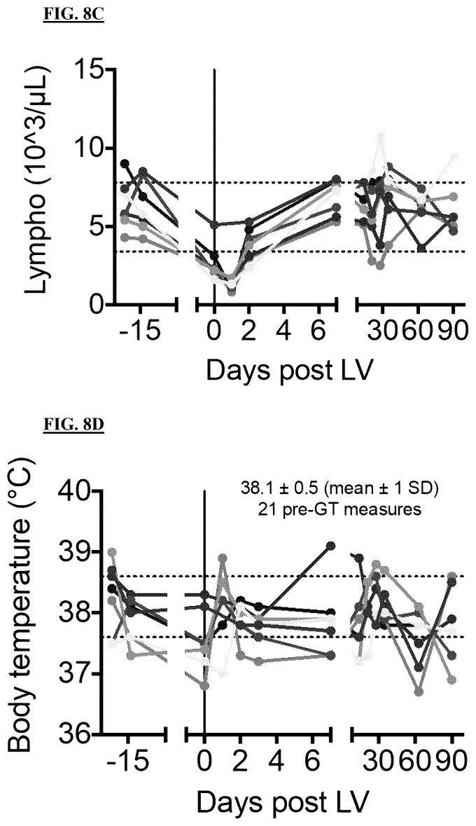

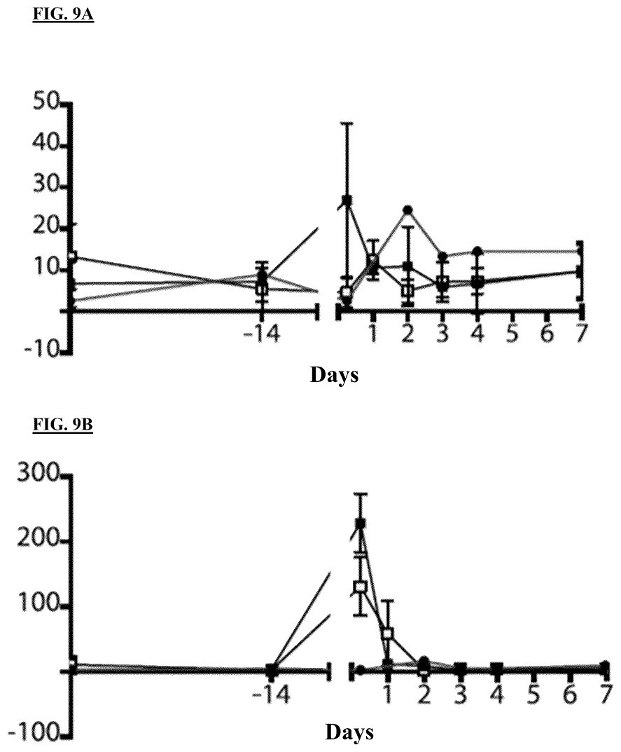

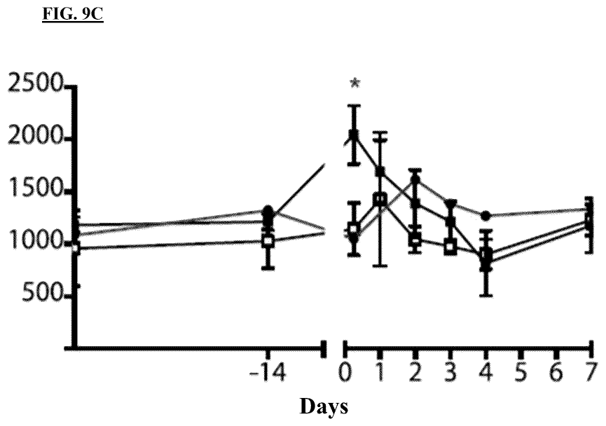

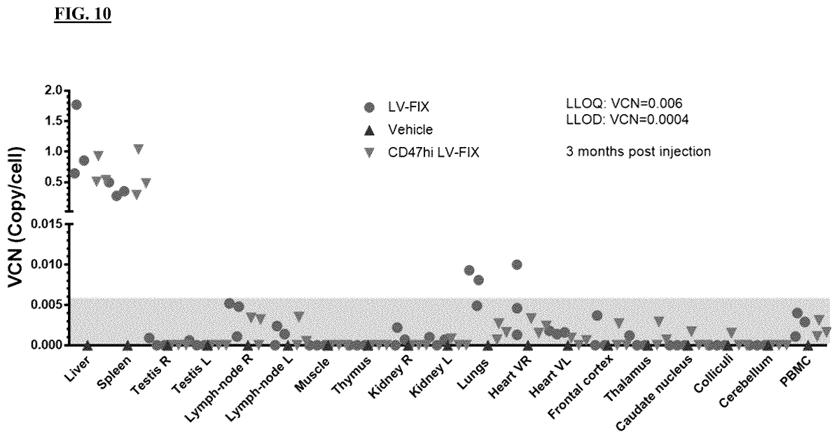

Animals administered LV-coFIX-1-R338L exhibited very mild acute immune responses, as indicated by ALT levels (FIG. 8A), AST levels (FIG. 8B), lympho levels (FIG. 8C) and body temperature (FIG. 8D) following administration. Decreased cytokine response was observed following administration of CD47^{high} LV-coFIX-1-R338L relative to the LV control vector (FIGS. 9A-9C). Whereas mild increases in MIP-1a, MIP-1b, and MCP-1 were observed following administration of control LV, MIP-1a, MIP-1b, and MCP-1 expression was lower and in some cases undetectably following administration of CD47^{high} LV-coFIX-1-R338L. As expected, the LV-coFIX-1-R338L localized primarily to the liver and spleen, having a vector copy number (VCN) in the liver and spleen that was greater than 100-fold higher than other organs (FIG. 10). These data suggest that CD47^{high} LV-coFIX-1-R338L induces a reduced allo-specific immune response, with increased resistance to phagocytosis and improved hepatocyte gene transfer.

### Example 5: Additional Testing of CD47High LV-coFIX-1-R338L Dose Response in Non-Human Primates

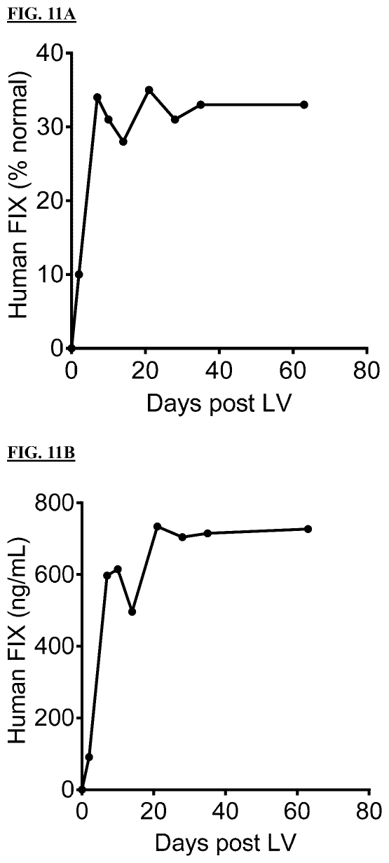

An additional Macaca nemestrina was treated with CD47^{high} LV-coFIX-1-R338L at 2.5E9 TU/kg dose via intravenous administration. Lentiviral vector-mediated human FIX expression was monitored by analyzing steady state circulated human FIX specific activity (FIG. 11A) and antigen level (FIG. 11B). Post LV treatment, the steady state circulating human FIX activity is about 33% of normal and the circulating human FIX antigen amount is at 700 ng/mL which correlated to 14% of normal FIX antigen level.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1-111. (canceled)

112. A method of treating hemophilia B in a human subject in need thereof comprising:
administering to the subject an effective dose of between 1×10⁹ and 8×10⁹ transducing units/kg (TU/kg) of a lentiviral vector,
wherein the lentiviral vector comprises:
a nucleotide sequence encoding a polypeptide with factor IX (FIX) activity comprising SEQ ID NO: 1, and
a lipid coat comprising at least 225 molecules/μm² to at least 400 molecules/μm² of a CD47 polypeptide;
wherein the lentiviral vector does not comprise an MHC-I polypeptide, and/or is produced in a host cell not expressing MHC-I; and
wherein the lentiviral vector at the effective dose induces a FIX activity of 10% to 100% of normal in the subject that lasts for at least 60 days after administration.

113. The method of claim 112, wherein the effective dose is less than 6×10⁹ TU/kg.

114. The method of claim 112, wherein the effective dose is less than 5×10⁹ TU/kg.

115. The method of claim 112, wherein the effective dose is less than 4×10⁹ TU/kg.

116. The method of claim 112, wherein the effective dose is less than 3×10⁹ TU/kg.

117. The method of claim 112, wherein the subject exhibits one or more of the following properties after administration of the composition:
(a) a decreased macrophage transduction of the lentiviral vector relative to a control lentiviral vector;
(b) a reduced allo-specific immune response to the lentiviral vector relative to the control lentiviral vector;
(c) a FIX activity of at least 30% relative to normal FIX activity for at least 3 weeks after administration;
(d) a tissue specific expression of the lentiviral vector in liver, spleen, or both the liver and spleen of the subject; or
(e) any combination of (a)-(d).

118. The method of claim 117, wherein the reduced allo-specific immune response comprises the release of a cytokine in response to the lentiviral vector.

119. The method of claim 118, wherein the cytokine is selected from the group consisting of MIP-1a, MIP-1b, MCP-1, and any combination thereof.

120. The method of claim 112, wherein the lentiviral vector provides increased FIX activity in the subject at 24 hours to 48 hours post administration of the lentiviral vector.

121. The method of claim 112, wherein the lentiviral vector exhibits an increased localization to liver, spleen, or both the liver and the spleen in the subject after administration, relative to an organ other than the liver and the spleen in the subject.

122. The method of claim 121, wherein the increased localization is characterized by a vector copy number (VCN) of the lentiviral vector that is at least 5-fold higher in the liver, spleen, or both the liver and the spleen following administration of the lentiviral vector, relative to an organ other than the liver and spleen in the subject.

123. The method of claim 112, wherein the polypeptide with FIX activity comprises the amino acid sequence of SEQ ID NO: 12.

124. The method of claim 112, wherein the lentiviral vector comprises a tissue specific promoter.

125. The method of claim 124, wherein the tissue specific promoter selectively enhances expression of the polypeptide with FIX activity in a target liver cell.

126. The method of claim 125, wherein the target liver cell is a hepatocyte.

127. The method of claim 124, wherein the tissue specific promoter comprises an APOA2 promoter, SERPINA1 (hAAT) promoter, mTTR promoter, MIR122 promoter, or any combination thereof.

128. The method of claim 112, wherein:
the lentiviral vector further comprises a splice acceptor site;
the lentiviral vector further comprises a propeptide sequence, a pol sequence, a rev sequence, a rev responsive element (RRE), or any combination thereof; and/or
the lentiviral vector further comprises an enhancer, a target sequence for a microRNA, a post-transcriptional regulatory element, a packaging signal, a poly-A sequence, an intron sequence, or any combination thereof.

129. The method of claim 112, wherein the FIX polypeptide is a R338L variant FIX polypeptide.

130. The method of claim 112, wherein the nucleotide sequence encoding a polypeptide with FIX activity further comprises a heterologous nucleotide sequence encoding a heterologous amino acid sequence, wherein:
the heterologous amino acid sequence is an albumin, an immunoglobulin Fc region, an XTEN sequence, the C-terminal peptide (CTP) of the R subunit of human chorionic gonadotropin, a PAS sequence, a HAP sequence, a CTP peptide sequence, a transferrin, albumin-binding moiety, or any fragments, derivatives, variants, or combinations of these polypeptides; and
the heterologous amino acid sequence is linked to the N-terminus or the C-terminus of the amino acid sequence encoded by a nucleotide sequence encoding a polypeptide with FIX activity, or inserted between two amino acids in the amino acid sequence.
